(19) United States
(12) Patent Application Publication
(10) Pub. No.: US 2012/0040991 A1
(43) Pub. Date: Feb. 16, 2012

(54) 3-AZABICYCLO [4.1.0] HEPTANES USED AS OREXIN ANTAGONISTS

(75) Inventors: David Amantini, Verona (IT); Romano Di Fabio, Verona (IT); Massimo Gianotti, Verona (IT); Francesca Pavone, Verona (IT); Francesco Ferroni, Verona (IT)

(73) Assignee: Glaxo Group Limited, Greenford Middlesex (GB)

(21) Appl. No.: 13/265,915

(22) PCT Filed: Apr. 23, 2010
(86) PCT No.: PCT/EP10/55449

§ 371 Date: Oct. 24, 2011

(30) Foreign Application Priority Data
Apr. 24, 2009 (GB) 0907112.7
Jun. 17, 2009 (GB) 0910483.7
Dec. 22, 2009 (GB) 0922472.6

(51) Int. Cl. A61K 31/444; A61K 31/506; C07D 413/14; A61K 31/497; A61P 25/24; A61P 25/00; A61P 9/00; A61P 11/00; A61P 25/22; C07D 401/14; A61K 31/501

(52) U.S. Cl. 514/252.02; 546/112; 514/299; 544/333; 514/256; 544/295; 514/255.05; 544/296; 544/238; 544/405; 514/252.01; 544/316; 514/274

(57) ABSTRACT

Disclosed are 3-azabicyclo[4.1.0]heptane derivatives of formula (I) and their use as orexin receptor antagonists.

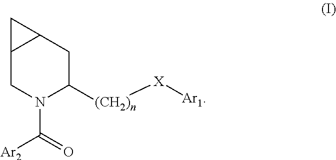

This invention relates to 3-azabicyclo[4.1.0]heptane derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711, WO08/038,251, WO09/003,993 and WO09/003,997 all disclose cyclic amine derivatives.

The compounds of the present invention have good bioavailability and brain penetration.

The present invention provides a compound of formula (I)

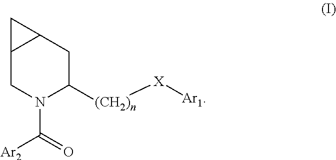

where

### X is O or S;

n is 1 or 2;
Ar₁ is a 5 or 6-membered monocyclic aromatic group having 0, 1, 2 or 3 nitrogen atoms, which group is optionally substituted with 1 or 2 groups independently selected from C₁₋₄alkyl, C₁₋₄alkoxy, haloC₁₋₄alkyl, haloC₁₋₄alkoxy, halo or cyano; or Ar1 is an 8 to 10 membered bicyclic heterocyclyl group having 1, 2 or 3 heteroatoms selected from N, O or S which bicyclic heterocyclyl group is optionally substituted with C₁₋₄alkyl, haloC₁₋₄alkyl or halo;
Ar₂ is a group selected from phenyl, pyridinyl, pyrimidinyl, pyridazinyl, pyrazinyl or thiazolyl which group is substituted with 1 or 2 groups independently selected from C₁₋₄alkyl, C₁₋₄alkoxy, haloC₁₋₄alkyl, haloC₁₋₄alkoxy, cyano or a group Y;
Y is a group selected from phenyl, phenyloxy, pyridyl, pyrimidinyl, pyridazinyl, pyrazinyl, oxadiazolyl or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;
or a pharmaceutically acceptable salt thereof.

In one embodiment the invention provides a compound of formula (I)

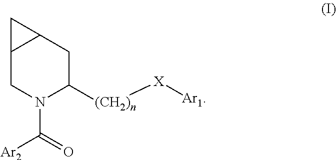

where

### X is O or S;

n is 1 or 2;
Ar₁ is a 5 or 6-membered monocyclic aromatic group having 0, 1, 2 or 3 nitrogen atoms, which group is optionally substituted with 1 or 2 groups independently selected from C₁₋₄alkyl, C₁₋₄alkoxy, haloC₁₋₄alkyl, haloC₁₋₄alkoxy, halo or cyano; or Ar1 is an 8 to 10 membered bicyclic heterocyclyl group having 1, 2 or 3 heteroatoms selected from N, O or S which bicyclic heterocyclyl group is optionally substituted with C₁₋₄alkyl, haloC₁₋₄alkyl or halo;
Ar₂ is a group selected from phenyl, pyridinyl, pyrimidinyl, pyridazinyl, pyrazinyl or thiazolyl wherein said group is substituted with a group selected from C₁₋₄alkyl, C₁₋₄alkoxy, haloC₁₋₄alkyl, haloC₁₋₄alkoxy, cyano and is additionally substituted with a group Y where Y is a group selected from phenyl, phenyloxy, pyridinyl, pyrimidinyl, pyridazinyl, pyrazinyl, oxadiazolyl or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;
or a pharmaceutically acceptable salt thereof.

The present invention also provides a compound of formula (I)

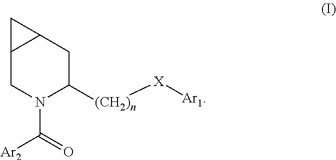

where

### X is O or S;

n is 1 or 2;
Ar₁ is a 5 or 6-membered monocyclic aromatic group having 0, 1, 2 or 3 nitrogen atoms, which group is optionally substituted with 1 or 2 groups independently selected from C₁₋₄alkyl, C₁₋₄alkoxy, haloC₁₋₄alkyl, haloC₁₋₄alkoxy, halo or cyano; or Ar1 is an 8 to 10 membered bicyclic heterocyclyl group which bicyclic heterocyclyl group is optionally substituted with C₁₋₄alkyl, haloC₁₋₄alkyl or halo;
Ar₂ is a group selected from phenyl, pyridinyl, pyrimidinyl, pyridazinyl, pyrazinyl or thiazolyl which group is substituted with 1 or 2 groups independently selected from C₁₋₄alkyl, C₁₋₄alkoxy, haloC₁₋₄alkyl, haloC₁₋₄alkoxy, cyano or a group Y;
Y is a group selected from phenyl, phenyloxy, pyridyl, pyrimidinyl, pyridazinyl, pyrazinyl, oxadiazolyl or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;
or a pharmaceutically acceptable salt thereof.

In one embodiment the invention provides a compound of formula (I)

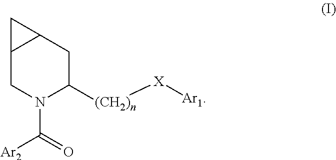

where

### X is O or S;

n is 1 or 2;
Ar₁ is a 5 or 6-membered monocyclic aromatic group having 0, 1, 2 or 3 nitrogen atoms, which group is optionally substituted with 1 or 2 groups independently selected from C₁₋₄alkyl, C₁₋₄alkoxy, haloC₁₋₄alkyl, haloC₁₋₄alkoxy, halo or cyano; or Ar1 is an 8 to 10 membered bicyclic heterocyclyl group which bicyclic heterocyclyl group is optionally substituted with C₁₋₄alkyl, haloC₁₋₄alkyl or halo;
Ar₂ is a group selected from phenyl, pyridinyl, pyrimidinyl, pyridazinyl, pyrazinyl or thiazolyl wherein said group is substituted with a group selected from C₁₋₄alkyl, C₁₋₄alkoxy, haloC₁₋₄alkyl, haloC₁₋₄alkoxy, cyano and is additionally substituted with a group Y where Y is a group selected from phenyl, phenyloxy, pyridinyl, pyrimidinyl, pyridazinyl, pyrazinyl, oxadiazolyl or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;
or a pharmaceutically acceptable salt thereof.

In one embodiment X is O.

In one embodiment the compounds of the invention are in a trans (1R,4S,6R)-configuration (formula (II)).

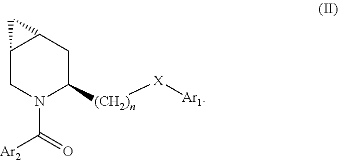

wherein the features X, n, Ar₁ and Ar₂ are as defined for formula (I).

In one embodiment X is O.

In one embodiment Ar₁ is pyridinyl.

In another embodiment Ar₁ is pyrimidinyl.

In one embodiment Ar₂ is pyridinyl.

In one embodiment Ar₂ is pyridinyl substituted with the group methyl and with a group selected from ethoxy, propoxy, phenyl, triazolyl, oxazolyl, thiazolyl, oxadiazolyl or pyrimidinyl.

In one embodiment Ar₁ is substituted with —CF₃.

In one embodiment both Ar₁ and Ar₂ are pyridinyl.

In one embodiment Ar₁ is pyridinyl substituted with —CF₃ and Ar₂ is pyridinyl substituted with the group methyl and with a group selected from phenyl, triazolyl, oxazolyl, thiazolyl, oxadiazolyl or pyrimidinyl.

In one embodiment the invention provides a compound of formula (II)

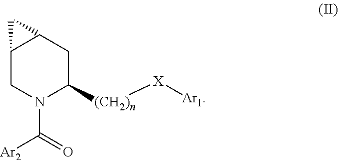

where

### X is O;

n is 1;
Ar₁ is a pyridinyl, pyrimidinyl or pyridazinyl group, which group is optionally substituted with 1 or 2 groups independently selected from C₁₋₄alkyl, C₁₋₄alkoxy, haloC₁₋₄alkyl, haloC₁₋₄alkoxy, halo or cyano;
Ar₂ is pyridinyl or pyrimidinyl wherein said pyridinyl or pyrimidinyl group is substituted with C₁₋₄alkyl and is additionally substituted with a group Y where Y is a group selected from phenyl, pyrazolyl, triazolyl or pyrimidinyl, which group Y is optionally substituted with C₁₋₄alkyl;
or a pharmaceutically acceptable salt thereof.

In one embodiment the invention provides a compound of formula (II)

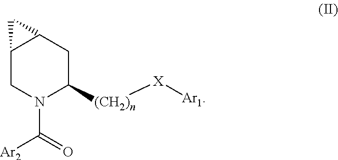

where

### X is O;

n is 1;
Ar₁ is a pyridinyl, pyrimidinyl or pyridazinyl group, which group is optionally substituted with 1 or 2 groups independently selected from methyl, methoxy, trifluoromethyl, fluoro, chloro or cyano;
Ar₂ is pyridinyl substituted with methyl and a group Y where Y is a group selected from phenyl, pyrazolyl, triazolyl or pyrimidinyl, which group Y is optionally substituted with methyl;
or a pharmaceutically acceptable salt thereof.

In one embodiment the compounds of the invention are in a cis (1S,4S,6S)-configuration (formula (III)).

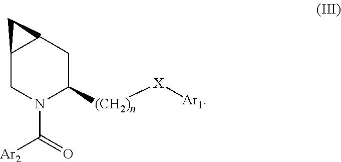

wherein the features X, n, Ar₁ and Ar₂ are as defined for formula (I).

In one embodiment X is O.

In one embodiment Ar₁ is pyridinyl.

In another embodiment Ar₁ is pyrimidinyl.

In one embodiment Ar₂ is pyridinyl.

In one embodiment Ar₂ is pyridinyl substituted with the group methyl and with a group selected from ethoxy, propoxy, phenyl, triazolyl, oxazolyl, thiazolyl, oxadiazolyl or pyrimidinyl.

In one embodiment Ar₁ is substituted with —CF₃.

In one embodiment both Ar₁ and Ar₂ are pyridinyl.

In one embodiment Ar₁ is pyridinyl substituted with —CF₃ and Ar₂ is pyridinyl substituted with the group methyl and with a group selected from phenyl, triazolyl, oxazolyl, thiazolyl, oxadiazolyl or pyrimidinyl.

In one embodiment the invention provides a compound of formula (III)

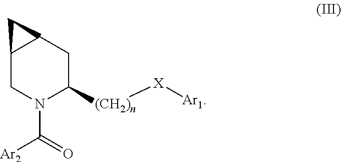

where

### X is O;

n is 1;
Ar₁ is a pyridinyl, pyrimidinyl or pyridazinyl group, which group is optionally substituted with 1 or 2 groups independently selected from C₁₋₄alkyl, C₁₋₄alkoxy, haloC₁₋₄alkyl, haloC₁₋₄alkoxy, halo or cyano;
Ar₂ is pyridinyl or pyrimidinyl wherein said pyridinyl or pyrimidinyl group is substituted with C₁₋₄alkyl and is additionally substituted with a group Y where Y is a group selected from phenyl, pyrazolyl, triazolyl or pyrimidinyl, which group Y is optionally substituted with C₁₋₄alkyl;
or a pharmaceutically acceptable salt thereof.

In one embodiment the invention provides a compound of formula (III)

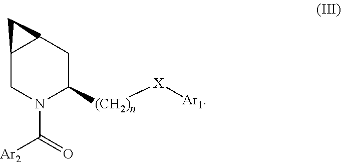

where

### X is O;

n is 1;
Ar₁ is a pyridinyl, pyrimidinyl or pyridazinyl group, which group is optionally substituted with 1 or 2 groups independently selected from methyl, methoxy, trifluoromethyl, fluoro, chloro or cyano;
Ar₂ is pyridinyl substituted with methyl and a group Y where Y is a group selected from phenyl, pyrazolyl, triazolyl or pyrimidinyl, which group Y is optionally substituted with methyl;

In one embodiment the invention provides the compound of formula (I) selected from the group consisting of:
(1R,4S,6R)-3-{[6-Methyl-3-(propyloxy)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyrimidinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyrimidinyl]oxy}methyl)-3-azabicyclo[4.1.0]heptane;
(1R,4S,6R)-3-[(6-methyl-3-phenyl-2-pyridinyl)carbonyl]-4-({[5-(trifluoromethyl)-2-pyrimidinyl]oxy}methyl)-3-azabicyclo[4.1.0]heptane;
(1R,4S,6R)-4-({[5-(methyloxy)-2-pyrimidinyl]oxy}methyl)-3-[(6-methyl-3-phenyl-2-pyridinyl)carbonyl]-3-azabicyclo[4.1.0]heptane;
(1R,4S,6R)-3-[(6-methyl-3-phenyl-2-pyridinyl)carbonyl]-4-({[6-(trifluoromethyl)-3-pyridazinyl]oxy}methyl)-3-azabicyclo[4.1.0]heptane;
(1R,4S,6R)-3-{[6-methyl-3-(2-pyrimidinyl)-2-pyridinyl]carbonyl}-4-({[6-(trifluoromethyl)-2-pyridinyl]oxy}methyl)-3-azabicyclo[4.1.0]heptane;
(1R,4S,6R)-4-{[(5-chloro-3-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-({[3-fluoro-5-(trifluoromethyl)-2-pyridinyl]oxy}methyl)-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[3-(trifluoromethyl)-2-pyridinyl]oxy}methyl)-3-azabicyclo[4.1.0]heptane;
6-[({(1R,4S,6R)-3-[(6-methyl-3-phenyl-2-pyridinyl)carbonyl]-3-azabicyclo[4.1.0]hept-4-yl}methyl)oxy]-3-pyridinecarbonitrile;
(1R,4S,6R)-4-({[6-(methyloxy)-2-pyridinyl]oxy}methyl)-3-[(6-methyl-3-phenyl-2-pyridinyl)carbonyl]-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyrimidinyl]oxy}methyl)-3-azabicyclo[4.1.0]heptane;
(1R,4S,6R)-3-{[5-methyl-2-(2-pyrimidinyl)phenyl]carbonyl}-4-({[6-(trifluoromethyl)-3-pyridazinyl]oxy}methyl)-3-azabicyclo[4.1.0]heptane;
(1R,4S,6R)-4-{[(4,5-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(2,6-dichloro-4-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4,6-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[4-(trifluoromethyl)-2-pyrimidinyl]oxy}methyl)-3-azabicyclo[4.1.0]heptane;
(1R,4S,6R)-3-{[6-methyl-3-(2-pyrimidinyl)-2-pyridinyl]carbonyl}-4-({[2-(trifluoromethyl)-4-pyrimidinyl]oxy}methyl)-3-azabicyclo[4.1.0]heptane;
(1R,4S,6R)-3-{[6-methyl-3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[5-(trifluoromethyl)-2-pyrimidinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-{[(3-methyl-2-pyrazinyl)oxy]methyl}-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[6-(trifluoromethyl)-3-pyridazinyl]oxy}methyl)-3-azabicyclo[4.1.0]heptane;
2-[({(1R,4S,6R)-3-[(6-methyl-3-phenyl-2-pyridinyl)carbonyl]-3-azabicyclo[4.1.0]hept-4-yl}methyl)oxy]-1,3-benzoxazole;
(1R,4S,6R)-4-{[(5-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[6-methyl-4-(trifluoromethyl)-2-pyridinyl]oxy}methyl)-3-azabicyclo[4.1.0]heptane;
(1R,4S,6R)-4-{[(6-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(3,5-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(5-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(3-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(5-ethyl-1,3-oxazol-2-yl)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(4-methyl-1,3-thiazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyridinyl]oxy}methyl)-3-azabicyclo[4.1.0]heptane;
(1R,4S,6R)-3-[(6-methyl-3-phenyl-2-pyridinyl)carbonyl]-4-({[5-(trifluoromethyl)-2-pyridinyl]oxy}methyl)-3-azabicyclo[4.1.0]heptane;
(1R,4S,6R)-3-{[6-methyl-3-(1H-pyrazol-1-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyrazinyl]oxy}methyl)-3-azabicyclo[4.1.0]heptane;
3-methyl-1-{[(1R,4S,6R)-4-({[5-(trifluoromethyl)-2-pyridinyl]oxy}methyl)-3-azabicyclo[4.1.0]hept-3-yl]carbonyl}-5H-imidazo[5,1-a]isoindole;
(1R,4S,6R)-3-{[5-methyl-2-(2-pyrimidinyl)phenyl]carbonyl}-4-({[5-(trifluoromethyl)-2-pyridinyl]oxy}methyl)-3-azabicyclo[4.1.0]heptane;
(1R,4S,6R)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane;
(1S,4S,6S)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane;
(1S,4S,6S)-3-{[3-(2-pyrimidinyl)-2-pyridinyl]carbonyl}-4-({[4-(trifluoromethyl)-2-pyridinyl]oxy}methyl)-3-azabicyclo[4.1.0]heptane;
(1S,4S,6S)-3-{[5-methyl-2-(2-pyrimidinyl)phenyl]carbonyl}-4-({[4-(trifluoromethyl)-2-pyridinyl]oxy}methyl)-3-azabicyclo[4.1.0]heptane;
(1S,4S,6S)-4-{2-[(5-fluoro-2-pyridinyl)oxy]ethyl}-3-{[6-methyl-3-(2H-1,2,3-triazol-2-yl)-2-pyridinyl]carbonyl}-3-azabicyclo[4.1.0]heptane;
(1S,4S,6S)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane;
(1R,4S,6R)-3-[(6-methyl-2-pyridinyl)carbonyl]-4-({[5-(trifluoromethyl)-2-pyridinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyridinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyrimidinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyrazinyl]oxy}methyl)-3-azabicyclo[4.1.0]heptane;
(1R,4S,6R)-3-{[6-methyl-3-(2-pyrimidinyl)-2-pyridinyl]carbonyl}-4-({[6-(trifluoromethyl)-3-pyridazinyl]oxy}methyl)-3-azabicyclo[4.1.0]heptane;
2-methyl-6-{[((1R,4S,6R)-3-{[6-methyl-3-(2-pyrimidinyl)-2-pyridinyl]carbonyl}-3-azabicyclo[4.1.0]hept-4-yl)methyl]oxy}-3-pyridinecarbonitrile;
(1R,4S,6R)-4-{[(4,6-dimethyl-2-pyrimidinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(5,6-dimethyl-2-pyrazinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-(2-{[5-(trifluoromethyl)-2-pyridinyl]oxy}ethyl)-3-azabicyclo[4.1.0]heptane;
(1R,4S,6R)-3-[(3-chloro-6-methyl-2-pyridinyl)carbonyl]-4-{2-[(5-fluoro-2-pyridinyl)oxy]ethyl}-3-azabicyclo[4.1.0]heptane;
(1R,4S,6R)-4-{2-[(4,6-dimethyl-2-pyrimidinyl)oxy]ethyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-[2-(3-pyridinyloxy)ethyl]-3-azabicyclo[4.1.0]heptane;
(1S,4S,6S)-4-{2-[(5-fluoro-2-pyridinyl)oxy]ethyl}-3-{[6-methyl-3-(1-pyrazol-1-yl)-2-pyridinyl]carbonyl}-3-azabicyclo[4.1.0]heptane;
(1S,4S,6S)-3-{[6-methyl-3-(2-pyrimidinyl)-2-pyridinyl]carbonyl}-4-({[4-methyl-5-(trifluoromethyl)-2-pyridinyl]oxy}methyl)-3-azabicyclo[4.1.0]heptane;
(1S,4S,6S)-3-{[6-methyl-3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1S,4S,6S)-4-{[(2,6-dichloro-4-pyridinyl)oxy]methyl}-3-{[6-methyl-3-(2-pyrimidinyl)-2-pyridinyl]carbonyl}-3-azabicyclo[4.1.0]heptane;
(1S,4S,6S)-4-{[(4,6-dichloro-2-pyridinyl)oxy]methyl}-3-{[6-methyl-3-(2-pyrimidinyl)-2-pyridinyl]carbonyl}-3-azabicyclo[4.1.0]heptane;
(1S,4S,6S)-4-{[(4,6-dichloro-2-pyridinyl)oxy]methyl}-3-{[3-(2-pyrimidinyl)-2-pyridinyl]carbonyl}-3-azabicyclo[4.1.0]heptane;
(1S,4S,6S)-4-{[(4-chloro-2-pyridinyl)oxy]methyl}-3-{[6-methyl-3-(2-pyrimidinyl)-2-pyridinyl]carbonyl}-3-azabicyclo[4.1.0]heptane;
(1S,4S,6S)-4-{[(4-chloro-2-pyridinyl)oxy]methyl}-3-{[3-(2-pyrimidinyl)-2-pyridinyl]carbonyl}-3-azabicyclo[4.1.0]heptane;
(1S,4S,6S)-3-{[3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1S,4S,6S)-3-{[6-methyl-3-(2-pyrimidinyl)-2-pyridinyl]carbonyl}-4-({[5-(trifluoromethyl)-2-pyrazinyl]oxy}methyl)-3-azabicyclo[4.1.0]heptane;
(1R,4S,6R)-4-{[(5-fluoro-2-pyridinyl)oxy]methyl}-3-[(2-methyl-5-phenyl-1,3-thiazol-4-yl)carbonyl]-3-azabicyclo[4.1.0]heptane;
(1R,4S,6R)-4-{[(4-fluoro-2-pyridinyl)oxy]methyl}-3-[(2-methyl-5-phenyl-1,3-thiazol-4-yl)carbonyl]-3-azabicyclo[4.1.0]heptane;
(1R,4S,6R)-4-{[(6-fluoro-2-pyridinyl)oxy]methyl}-3-[(2-methyl-5-phenyl-1,3-thiazol-4-yl)carbonyl]-3-azabicyclo[4.1.0]heptane; and
(1R,4S,6R)-4-{[(5-chloro-3-fluoro-2-pyridinyl)oxy]methyl}-3-[(2-methyl-5-phenyl-1,3-thiazol-4-yl)carbonyl]-3-azabicyclo[4.1.0]heptane;
   or a pharmaceutically acceptable salt thereof.

When the compound contains a C₁₋₄alkyl group, whether alone or forming part of a larger group, e.g. C₁₋₄alkoxy, the alkyl group may be straight chain, branched or cyclic, or combinations thereof. Examples of C₁₋₄alkyl are methyl or ethyl. An example of C₁₋₄alkoxy is methoxy.

Examples of haloC₁₋₄alkyl include trifluoromethyl (i.e. —CF₃).

Examples of C₁₋₄alkoxy include methoxy and ethoxy.

Examples of haloC₁₋₄alkoxy include trifluoromethoxy (i.e. —OCF₃).

Halogen or “halo” (when used, for example, in haloC₁₋₄)alkyl means fluoro, chloro, bromo or iodo.

Examples of a 5 or 6 membered monocyclic aromatic group containing 0, 1, 2 or 3 nitrogen atoms include phenyl, imidazolyl, pyrimidinyl, triazolyl, pyrrolyl, pyrazolinyl, pyridazinyl, pyrazinyl or pyridinyl.

Examples of a 5 or 6 membered heterocyclyl group containing 1, 2, 3 or 4 heteroatoms selected from N, O or S include pyrimidinyl, oxadiazolyl, oxazolyl, isoxazolyl, thiazolyl, triazolyl, imidazolyl, pyrrolyl, pyrazolinyl, pyridazinyl, pyrazinyl, pyridinyl, thienyl, furanyl, isothiazolyl or tetrazolyl.

Examples of an 8 to 10 membered bicyclic heterocyclyl group having 1, 2 or 3 heteroatoms selected from N, O or S include quinoxalinyl, quinazolinyl, pyridopyrazinyl, benzoxazolyl, benzothienyl, benzofuranyl, benzimidazolyl, naphthyridinyl, benzothiazolyl, indolyl, furopyridinyl, pyridopyrimidinyl, isoquinolinyl, quinolinyl, oxazolylpyridinyl, tetrahydrobenzimidazolyl or tetrahydrobenzofuranyl.

It is to be understood that the present invention covers all combinations of particularised groups and substituents described herein above.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

It will be understood that the invention includes pharmaceutically acceptable derivatives of compounds of formula (I) and that these are included within the scope of the invention.

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

In one embodiment the compounds of formula (I) are racemic. In another embodiment the compounds have the 4S configuration. Where additional chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) or salts thereof.

The following schemes are examples of synthetic schemes that may be used to synthesise compounds of the invention where X is O.

Compounds where n=1 can be made using the process shown in schemes 1, 2 or 4.

Compounds where n=2 can be made using the process shown in scheme 3 or 5.

Schemes 1, 2 and 3 show the synthesis of compounds of the invention that are in the trans (1R,4S,6R)-configuration. Examples of schemes for the synthesis of compounds that are in the cis (1S,4S,6S)-configuration are shown in schemes 4 and 5.

It will be apparent to the person skilled in the art that using the following schemes, and using an appropriate alternative intermediate, it will be possible to prepare compounds where X is S.

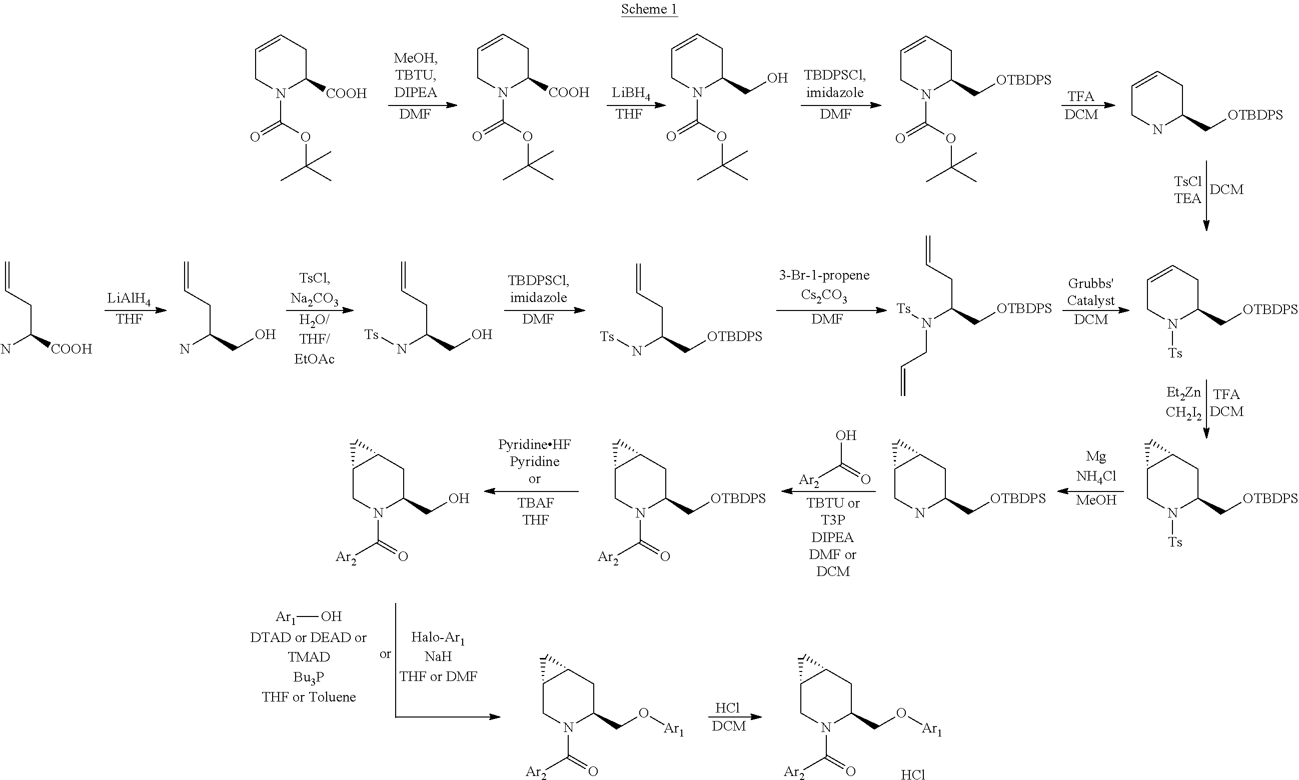

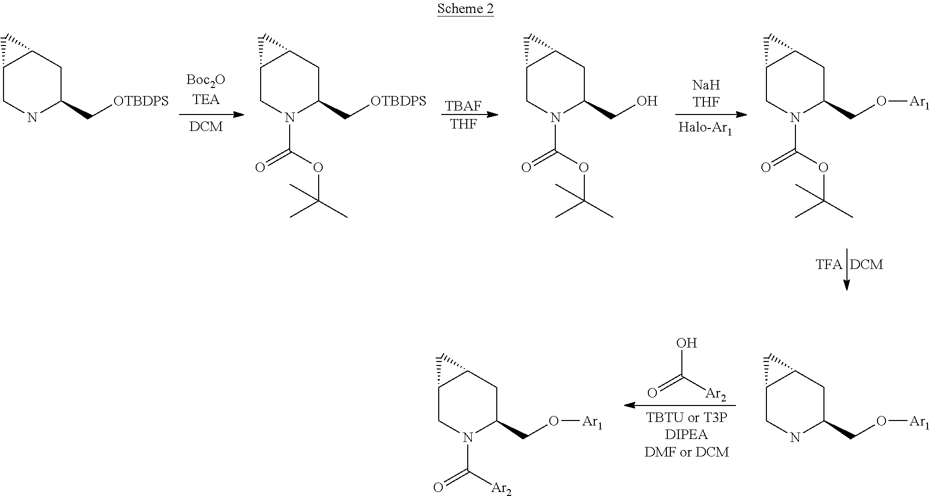

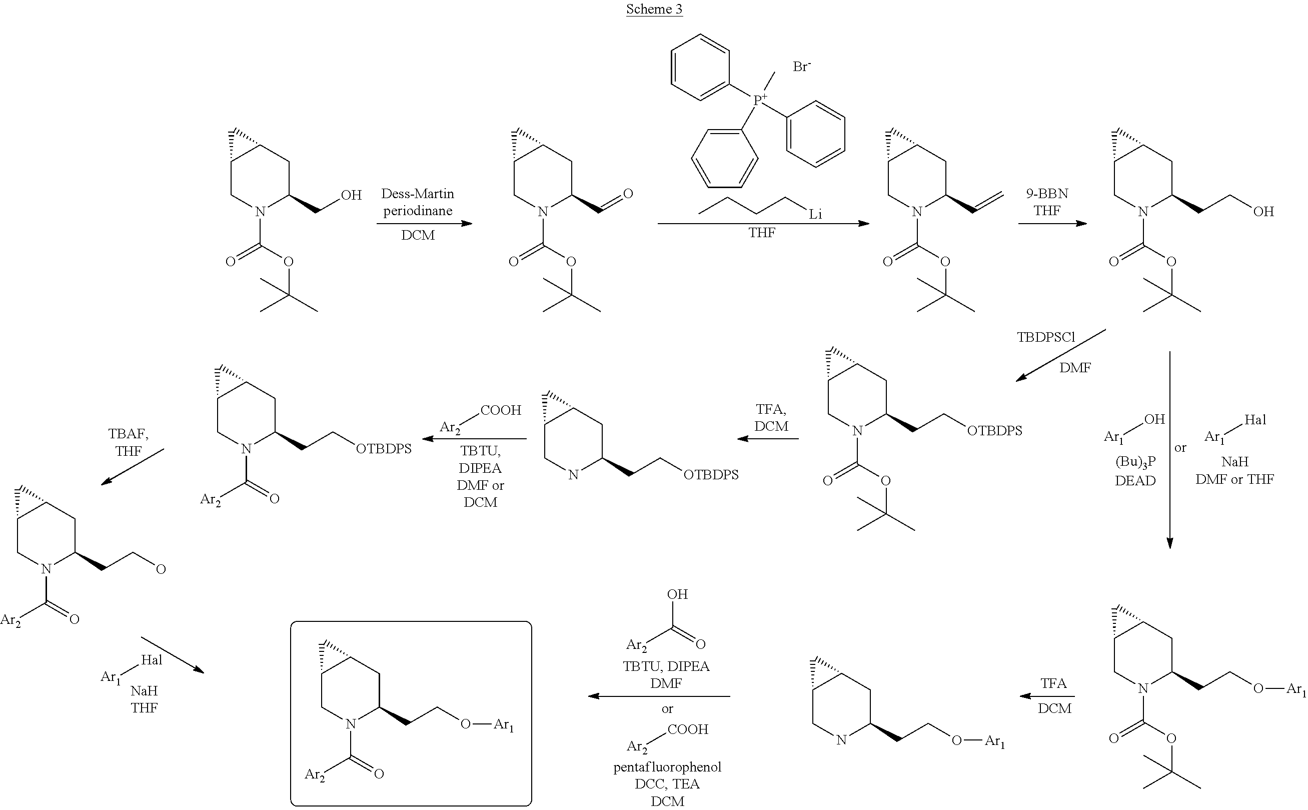

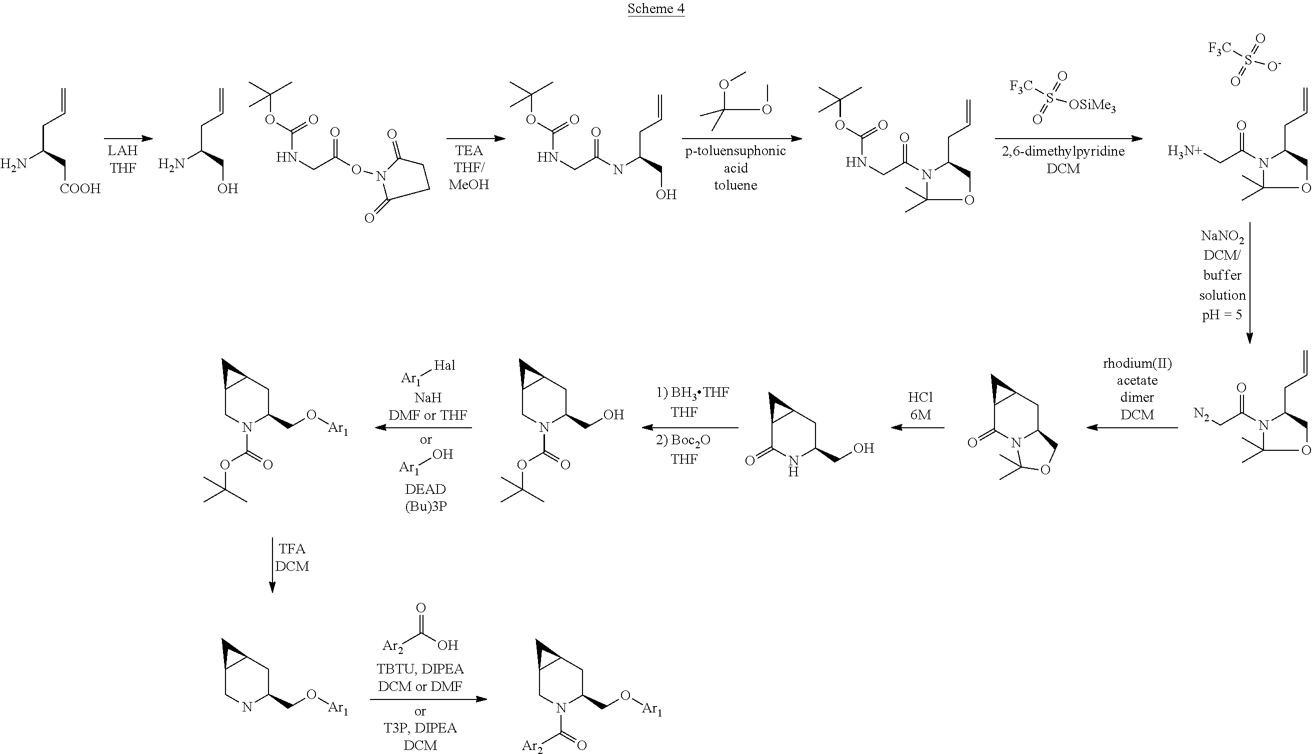

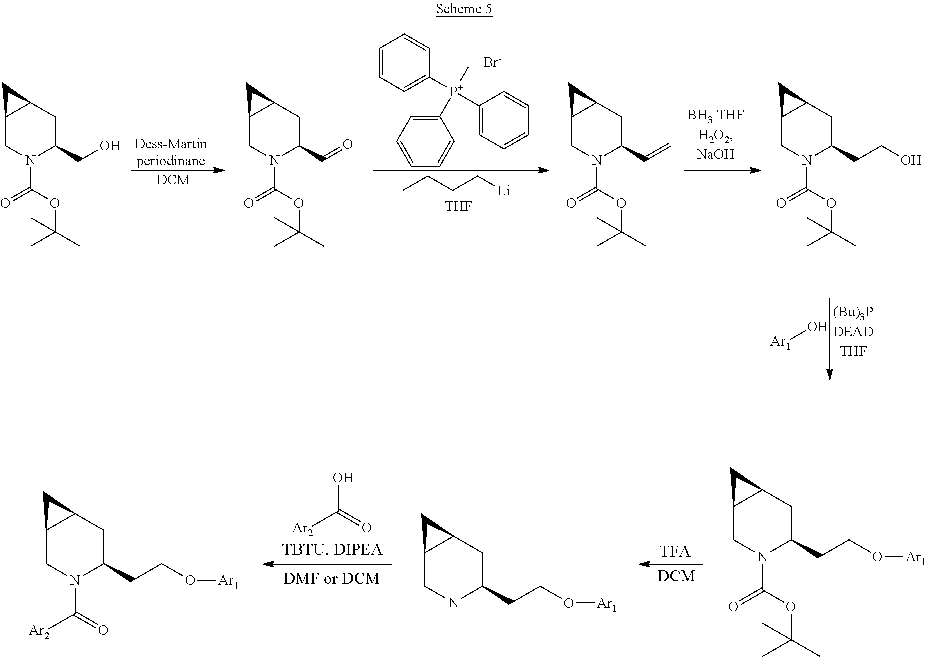

It will be understood by those skilled in the art that certain compounds of the invention can be converted into other compounds of the invention according to standard chemical methods.

The starting materials for use in the scheme are commercially available, known in the literature or can be prepared by known methods.

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the compounds of formula (I) or their pharmaceutically acceptable salts may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of a disease or disorder where an antagonist of a human orexin receptor is required such as Eating disorders include Anorexia Nervosa (307.1) including the subtypes Restricting Type and Binge-Eating/Purging Type; Bulimia Nervosa (307.51) including the subtypes Purging Type and Nonpurging Type; Obesity, including obesity observed in Type 2 (non-insulin-dependent) diabetes patients; Compulsive Eating Disorder; Binge Eating Disorder; and Eating Disorder Not Otherwise Specified (307.50).

Further, the compounds of formula (I) or their pharmaceutically acceptable salts may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method for the treatment of a disease or disorder where an antagonist of a human orexin receptor is required, for example those diseases and disorders mentioned hereinabove, in a subject in need thereof, comprising administering to said subject an effective amount of a compound of formula (I), or a pharmaceutically acceptable salt thereof.

The invention also provides a compound of formula (I), or a pharmaceutically acceptable salt thereof, for use in the treatment or prophylaxis of a disease or disorder where an antagonist of a human orexin receptor is required, for example those diseases and disorders mentioned hereinabove.

The invention also provides the use of a compound of formula (I), or a pharmaceutically acceptable salt thereof, in the manufacture of a medicament for use in the treatment or prophylaxis of a disease or disorder where an antagonist of a human Orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, and such unit doses may be administered more than once a day, for example two or three a day. Such therapy may extend for a number of weeks or months.

Orexin-A (Sakurai, T. et al (1998) Cell, 92 pp 573-585) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 96 illustrate the preparation of intermediates used to make compounds of formula (I) or salts thereof (Examples 1 to 56). The Descriptions 97 to 124 illustrate the preparation of intermediates used to make compounds of formula (I) or salts thereof (Examples 57 to 71).

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples described hereinafter have all been prepared as a first step from stereo chemically pure starting materials. The stereochemistry of the compounds of the Descriptions and Examples have been assigned on the assumption that the pure configuration of these centres are retained.

Compounds are named using ACD/Name PRO6.02 chemical naming software (Advanced Chemistry Development Inc., Toronto, Ontario, M5H2L3, Canada).

Proton Magnetic Resonance (NMR) spectra were recorded either on Varian instruments at 300, 400, 500 or 600 MHz, or on Bruker instruments at 400 MHz. Mono-(1H and 1H with homonuclear decoupling) and two-dimensional techniques (1H-1H COSY, 1H-1H ROESY, 1H-13C HSQC) were used for stereochemistry investigation. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

Unless otherwise specified, HPLC analyses indicated by HPLC (walk-up): rt (retention time)=x min, were performed on a Agilent 1100 series instrument using a Luna 3 u C18(2) 100A column (50×2.0 mm, 3 μm particle size) [Mobile phase and Gradient: 100% (water+0.05% TFA) to 95% (acetonitrile+0.05% TFA) in 8 min. Column T=40° C. Flow rate=1 mL/min. UV detection wavelength=220 nm]. Other HPLC analyses, indicated by HPLC (walk-up, 3 min method), were performed using an Agilent Zorbax SB-C18 column (50×3.0 mm, 1.8 μm particle size) [Mobile phase and Gradient: 100% (water+0.05% TFA) to 95% (CH₃CN+0.05% TFA) in 2.5 min, hold 0.5 min. Column T=60° C. Flow rate=1.5 mL/min. UV detection wavelength=220 nm].

In the analytical characterization of the described compounds “MS” refers to Mass Spectra taken by Direct infusion Mass or to Mass Spectra associated with peaks taken by UPLC/MS or HPLC/MS analysis, where the Mass Spectrometer used is as mentioned below.

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50] or on an Agilent LC/MSD 1100 Mass Spectrometer coupled with HPLC instrument Agilent 1100 Series, operating in positive or negative electrospray ionization mode and in both acidic and basic gradient conditions [Acidic gradient LC/MS-ES (+ or −): analyses performed on a Supelcosil ABZ+Plus column (33×4.6 mm, 3 μm). Mobile phase: A—water+0.1% HCO₂H/B—CH₃CN. Gradient (standard method): t=0 min 0% (B), from 0% (B) to 95% (B) in 5 min lasting for 1.5 min, from 95% (B) to 0% (B) in 0.1 min, stop time 8.5 min. Column T=room temperature. Flow rate=1 mL/min. Gradient (fast method): t=0 min 0% (B), from 0% (B) to 95% (B) in 3 min lasting for 1 min, from 95% (B) to 0% (B) in 0.1 min, stop time 4.5 min. Column T=room temperature. Flow rate=2 mL/min.

Basic gradient LC/MS-ES (+ or −): analyses performed on a XTerra MS C18 column (30×4.6 mm, 2.5 μm). Mobile phase: A—5 mM aq. NH₄HCO₃+ammonia (pH 10)/B—CH₃CN. Gradient: t=0 min 0% (B), from 0% (B) to 50% (B) in 0.4 min, from 50% (B) to 95% (B) in 3.6 min lasting for 1 min, from 95% (B) to 0% (B) in 0.1 min, stop time 5.8 min. Column T=room temperature. Flow rate=1.5 mL/min].

Mass range ES (+ or −): 100-1000 amu. UV detection range: 220-350 nm. The usage of this methodology is indicated by “LC-MS” in the analytic characterization of the described compounds.

Total ion current (TIC) and DAD UV chromatographic traces together with MS and UV spectra associated with the peaks were taken on a UPLC/MS Acquity™ system equipped with 2996 PDA detector and coupled to a Waters Micromass ZQ™ mass spectrometer operating in positive or negative electrospray ionisation mode [LC/MS-ES (+ or −): analyses performed using an Acquity™ UPLC BEH C18 column (50×2.1 mm, 1.7 μm particle size). Mobile phase: A—water+0.1% HCO₂H/B—CH₃CN+0.06% or 0.1% HCO₂H. Gradient: t=0 min 3% B, t=1.5 min 100% B, t=1.9 min 100% B, t=2 min 3% B stop time 2 min. Column T=40° C. Flow rate=1.0 mL/min. Mass range: ES (+): 100-1000 amu or ES(+): 50-800 amu. ES (−): 100-800 amu. UV detection range: 210-350 nm. The usage of this methodology is indicated by “UPLC (IPQC)” in the analytic characterization of the described compounds.

[LC/MS-ES (+ or −): analyses performed using an Acquity™ UPLC BEH C18 column (50×2.1 mm, 1.7 μm particle size). Mobile phase: A—water+0.1% HCO₂H/B—CH₃CN+0.06% or 0.1% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu or ES(+): 50-800 amu, ES (−): 100-800 amu. UV detection range: 210-350 nm. The usage of this methodology is indicated by “UPLC (Acid QC_POS_{—}50-800 or Acid GEN_QC or Acid FINAL_QC)” in the analytic characterization of the described compounds.

[LC/MS-ES (+ or −): analyses performed using an Acquity™ UPLC BEH C18 column (50×2.1 mm, 1.7 μm particle size). Mobile phase: A—water+0.1% HCO₂H/B—CH₃CN+0.06% or 0.1% HCO₂H. Gradient: t=0 min 3% B, t=1.06 min 99% B, t=1.45 min 99% B, t=1.46 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “UPLC (Acid GEN_QC_SS)” in the analytic characterization of the described compounds.

Total ion current (TIC) and DAD UV chromatographic traces together with MS and UV spectra associated with the peaks were taken on a UPLC/MS Acquity™ system equipped with PDA detector and coupled to a Waters SQD mass spectrometer operating in positive and negative alternate electrospray ionisation mode [LC/MS-ES+/−: analyses performed using an Acquity™ UPLC BEH C18 column (50×2.1 mm, 1.7 μm particle size). Mobile phase: A—10 mM aqueous solution of NH₄HCO₃ (adjusted to pH 10 with ammonia)/B—CH₃CN. Gradient: t=0 min 3% B, t=1.06 min 99% B lasting for 0.39 min, t=1.46 min 3% B, stop time 1.5 min. Column T=40° C. Flow rate=1.0 mL/min. Mass range: ES (+): 100-1000 amu or ES (+): 50-800 amu. ES (−): 100-1000 amu. UV detection range: 220-350 nm. The usage of this methodology is indicated by “UPLC (Basic GEN_QC or Basic QC_{—}50_{—}800_POS)” in the analytic characterization of the described compounds.

Unless otherwise specified, Preparative LC-MS purifications were run on a MDAP (Mass Detector Auto Purification) Waters instrument (MDAP FractionLynx). [LC/MS-ES (+): analyses performed using a Gemini C18 AXIA column (50×21 mm, 5 μm particle size). Mobile phase A: NH₄HCO₃ sol. 10 mM, pH 10; B: CH₃CN. Flow rate: 17 mL/min. The gradient will be specified each time]. [Method 20 ml_ACID_GENERIC: Column: XTerra MS Prep C18 19×100 mm 5 um. Mobile phase: A: H₂O+0.1% HCO₂H, B: CH₃CN+0.1% HCO₂H. Gradient: t=0 min 10% B, t=1.00 min 10% B, t=13.00 min 95% B, t=16.00 min 95% B, t=16.10 min 10% B stop time 19.00 min. Flow rate=20 mL/min. Range Wavelegth 210-350 nm, resolution 1.2 nm]

Preparative HPLC were also performed using a Water XBridge C18 OBD column (100×19 mm, 5 μm). Mobile phase A: 10 mM ammonium bicarbonate+ammonia (pH 10); B: MeCN. Flow rate 17 mL/min, range wavelength: 220-350 nm

Method BASIC 1=Gradient: t=0 min 20% B, t=12 min 70% B, t=13 min 100% B, t=14 min 20% B. Injection volume: 300 μl, injection vehicle: DMSO/MeOH 1:1.

Method BASIC 2=Gradient: t=min 10% B, t=0.5 min 15% B, t=12.5 min 100% B, t=13 min 100% B, t=13.1 min 10% B. Injection volume: 550 μl, injection vehicle: MeOH.

Preparative LC-MS purifications were also run on a MDAP (Mass Detector Auto Purification) Waters instrument. The usage of this methodology is indicated by “Fraction Lynx”.

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP—NH prepacked flash cartridges, ISOLUTE NH₂ prepacked cartridges or ISCO RediSep Silica cartridges.

SPE-SCX cartridges are ion exchange solid phase extraction columns supplied by Varian. The eluent used with SPE-SCX cartridges is DCM and MeOH or only MeOH followed by 2 N ammonia solution in MeOH. The collected fractions are those eluted with the ammonia solution in MeOH.
ACN Acetonitrile
AcOH Acetic acid
bs or br.s broad signal
Boc t-Butoxycarbonyl
Burgess reagent Methyl N-(triethylammoniumsulphonyl)carbamate
9-BBN 9-Borabicyclo[3.3.1]nonan solution
CV Column volume
Cy Cyclohexane
DCE Dichloroethane
DCM Dichloromethane
DEAD Di (tert-butyl)azodicarboxylate
DIPEA N,N-Diisopropyl-N-ethylamine
DME Dimethoxyethane
DMF Dimethylformamide
DMSO Dimethylsulfoxide
Et₂O Diethylether
EtOAc Ethylacetate
EtOH Ethanol
iPrOH isopropanol
Grubbs 1^{st} generation Benzylidene-bis(tricyclohexylphosphine)dichlororuthenium
MCPBA m-chloroperbenzoic acid
MeOH Methanol
NBS N-bromosuccinimide
NCS N-chlorosuccinimide
NMP N-methylpyrrolidone
Ph Phenyl
pH=3 buffer solution Citric acid/NaOH/HCl in water solution available from Merck KGaA
rt retention time
T temperature
TBDPS tert-Butyl diphenylsilyl
TBTU 0-(benzotriazol-1-yl)-N,N,N′N′-tetramethyluronium tetrafluoroborate
t-Bu tert-Butyl
t-BuOH tert-butanol
TEA Triethylamine
TEMPO 2,2,6,6-tetramethylpiperidine 1-oxyl
T₃P 2,4,6-tripropyl-1,3,5,2,4,6-trioxatriphosphorinane 2,4,6-trioxide
TFA Trifluoroacetic acid
THF Tetrahydrofuran
TMAD Tetramethylazadicarboxamide
TMS Trimethylsilyl
Ts p-Toluensulfonyl

### Experimental Section for (1R,4S,6R) [4.1.0] Compounds (Trans) Referring to Scheme 1-3

### Descriptions

### Description 1:1-(1,1-dimethylethyl)2-methyl (2S)-3,6-dihydro-1,2(2H)-pyridinedicarboxylate (D1)

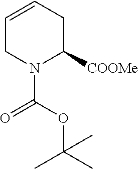

To a solution of (2S)-1-{[(1,1-dimethylethyl)oxy]carbonyl}-1,2,3,6-tetrahydro-2-pyridinecarboxylic acid (1.50 g, 6.60 mmol) in DMF (6 ml), DIPEA (6.92 ml, 39.60 mmol) and TBTU (2.97 g, 9.24 mmol) were added and the mixture stirred at room temperature for 45 minutes. MeOH (1.42 ml, 35.10 mmol) was added and the resulting reaction mixture stirred for 2 hours. The mixture was diluted with DCM and washed with a saturated NaHCO₃ aqueous solution. The organic layer was separated, dried (Na₂SO₄), filtered through a phase separator tube and concentrated under reduced pressure. The crude material was purified by flash chromatography on silica gel (Flash Master 70 g, Cy/EtOAc 90:10). Collected fractions gave the title compound D1 (1.10 g).

UPLC (Acid GEN_QC): rt=0.73 minutes, peak observed: 242 (M+1), 186 [M+1-C(Me)₃)] and 142 (M+1-Boc) C₁₂H₁₉NO₄ requires 241. ¹H-NMR (400 MHz, CDCl₃) δ(ppm): 5.60-5.82 (m, 2H), 4.84-5.15 (m, 1H), 4.01-4.19 (m, 1H), 3.75-3.89 (m, 1H), 3.69-3.76 (m, 3H), 2.44-2.72 (m, 2H), 1.45-1.55 (m, 9H).

### Description 2: 1,1-dimethylethyl (2S)-2-(hydroxymethyl)-3,6-dihydro-1(2H)-pyridinecarboxylate (D2)

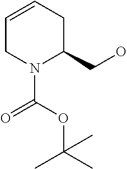

A solution of 1-(1,1-dimethylethyl)2-methyl (28)-3,6-dihydro-1,2(2H)-pyridinedicarboxylate D1 (1.10 g) in THF (25 ml) was cooled down to 0° C. and lithium borohydride (2.3 M solution in THF, 4.96 ml, 11.40 mmol) was added dropwise. The resulting reaction mixture was stirred at room temperature overnight. Further lithium borohydride (9.92 ml, 22.80 ml) was added, the mixture was stirred for 6 hours and then quenched with brine and extracted with EtOAc. The organic phase was separated, dried (Na₂SO₄), filtered through a phase separator tube and concentrated under reduced pressure to afford the title compound D2 (0.98 g). The material was used in the next step without any further purification.

UPLC (Acid GEN_QC: rt=0.59 minutes, peaks observed: 214 (M+1), 158 [M+1-C(Me)₃)] and 114 (M+1-Boc). C₁₁H₁₉NO₃ requires 213.

¹H-NMR (400 MHz, CDCl₃) δ(ppm): 5.61-5.82 (m, 2H), 4.35-4.64 (m, 1H), 3.98-4.30 (m, 1H), 3.48-3.73 (m, 3H), 2.35-2.48 (m, 1H), 1.96-2.15 (m, 1H), 1.50 (m, 9H).

### Description 3: 1,1-dimethylethyl (2S)-2-({[(1,1-dimethylethyl)(diphenyl)silyl]oxy}methyl)-3,6-dihydro-1(2H)-pyridinecarboxylate (D3)

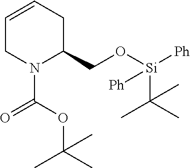

To a solution of 1,1-dimethylethyl (25)-2-(hydroxymethyl)-3,6-dihydro-1(2H)-pyridinecarboxylate D2 (0.98 g of the crude material obtained in the Description 2) in DMF (5 ml), imidazole (1.56 g, 22.97 mmol) and chloro(1,1-dimethylethyl)diphenylsilane (1.52 g, 5.52 mmol) were added and the reaction mixture was left under stirring at room temperature for 3 hours. The mixture was diluted with brine and extracted with EtOAc. The organic phase was separated, dried (Na₂SO₄), filtered through a phase separator tube and concentrated under reduced pressure. The residue was purified by flash chromatography on silica gel (Flash Master 70 g, Cy/EtOAc 90:10) to afford the title compound D3 (1.81 g). UPLC (Acid GEN_QC: rt=1.26 minutes, peaks observed: 452 (M+1) and 474 (M+Na). C₂₇H₃₇NO₃Si requires 451. ¹H-NMR (400 MHz, CDCl₃) δ(ppm): 7.57-7.78 (m, 4H), 7.32-7.51 (m, 6H), 5.44-5.75 (m, 2H), 4.37-4.80 (m, 1H), 4.02-4.31 (m, 1H), 3.53-3.72 (m, 2H), 3.28-3.51 (m, 1H), 1.99-2.44 (m, 2H), 1.48 (s, 9H), 1.07 (s, 9H).

### Description 4: (2S)-2-({[(1,1-dimethylethyl)(diphenyl)silyl]oxy}methyl)-1,2,3,6-tetrahydropyridine (D4)

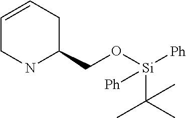

To a solution of 1,1-dimethylethyl (25)-2-({[(1,1-dimethylethyl)(diphenyl)silyl]oxy}methyl)-3,6-dihydro-1(2H)-pyridinecarboxylate D3 (1.81 g) in DCM (40 ml), TFA (20 ml) was added and the reaction mixture stirred at room temperature for 1 hour. Volatiles were removed under reduced pressure and the residue was eluted through a SCX column. Collected fractions gave the title compound D4 (1.35 g).

UPLC (Acid GEN_QC: rt=0.70 minutes, peak observed: 352 (M+1) C₂₂H₂₉NOSi requires 351. ¹H-NMR (300 MHz, CDCl₃) δ(ppm): 7.57-7.78 (m, 4H), 7.32-7.51 (m, 6H), 5.71-5.76 (m, 2H), 3.54-3.72 (m, 2H), 3.34-3.53 (m, 2H), 2.89-3.02 (m, 1H), 1.83-1.92 (m, 2H), 1.07 (s, 9H).

### Description 5A and 5B: (2S-2-({[(1,1-dimethylethyl)(diphenyl)silyl]oxy}methyl)-1-[(4-methylphenyl)sulfonyl]-1,2,3,6-tetrahydropyridine (D5A/D5B)

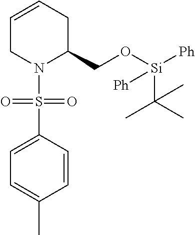

A) To a solution of (2S)-2-({[(1,1-dimethylethyl)(diphenyl)silyl]oxy}methyl)-1,2,3,6-tetrahydropyridine D4 (1.35 g) in DCM (25.60 ml), TEA (1.07 ml, 7.68 mmol) and 4-methylbenzenesulfonyl chloride (0.80 g, 4.22 mmol) were added and the resulting reaction mixture was stirred at room temperature overnight. The mixture was washed with a saturated aqueous NH₄Cl solution. The organic layer was separated, dried (Na₂SO₄), filtered through a phase separator tube and concentrated under reduced pressure. The residue was purified by flash chromatography on silica gel (Biotage SP, column size 40 M, from Cy 100 to Cy/EtOAc 90:10) to afford the title compound D5A (1.90 g).

UPLC (Acid GEN_QC): rt=1.18 minutes, peaks observed: 506 (M+1) and 528 (M+Na). C₂₉H₃₅NO₃SSi requires 505. ¹H-NMR (300 MHz, CDCl₃) δ(ppm): 7.29-7.76 (m, 12H), 7.15 (d, 2H), 5.45-5.67 (m, 2H), 4.42-4.37 (m, 1H), 3.92-4.11 (m, 1H), 3.51-3.61 (m, 2H), 3.35-3.50 (m, 1H), 2.37 (s, 3H), 2.04-2.33 (m, 2H), 1.03 (s, 9H).

B) N-[(15)-1-({[(1,1-dimethylethyl)(diphenyl)silyl]oxy}methyl)-3-buten-1-yl]-4-methyl-N-2-propen-1-ylbenzenesulfonamide D9 (7.46 g) was dissolved in DCM (50 ml) then Grubbs I (1.170 g, 1.398 mmol) was added and the mixture was stirred at room temperature overnight. All volatiles were removed under vacuum and the resulting crude product was purified by Silica Gel Chromatography (Biotage SP column size 340 g SNAP, Cy to Cy/EtOAc 80:20) to afford the title compound D5B (7.4 g).

### Description 6: (1R,4S,6R)-4-{[(1,1-dimethylethyl)(diphenyl)silyl]oxy}methyl)-3-[(4-methylphenyl)sulfonyl]-3-azabicyclo[4.1.0]heptane (D6)

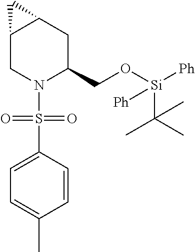

A solution of diethylzinc 1 M solution in hexane (21.35 ml, 21.35 mmol) in DCM (10 ml) was cooled down to 0° C. and TFA (1.64 ml, 21.35 mmol) was added dropwise. After 20 minutes stirring, diiodomethane (1.73 mol, 21.35 mmol) was added and the mixture left stirring for a further 20 minutes. A solution of (2S)-2-({[(1,1-dimethylethyl)(diphenyl)silyl]oxy}methyl)-1-[(4-methylphenyl)sulfonyl]-1,2,3,6-tetrahydropyridine D5A (1.35 g) in DCM (5 ml) was then added, the resulting reaction mixture was allowed to warm up to room temperature and stirred for 6 hours. A solution of diethylzinc (8 eq), TFA (8 eq) and diiodomethane (8 eq) in DCM was prepared and added to the previous mixture at 0° C. The resulting reaction mixture was left under stirring at room temperature overnight and washed with a saturated aqueous NH₄Cl solution. The aqueous layer was back-extracted with EtOAc. The collected organic layers were dried (Na₂SO₄), filtered through a phase separator tube and concentrated under reduced pressure. The residue was purified by flash chromatography on silica gel (Biotage SP, column size 40 M, from Cy 100 to Cy/EtOAc 90:10) to afford the title compound D6 (0.83 g).

UPLC (Acid GEN_QC): rt=1.22 minutes, peaks observed: 520 (M+1) and 542 (M+Na). C₃₀H₃₇NO₃SSi requires 519. ¹H-NMR (300 MHz, CDCl₃) δ(ppm): 7.50-7.75 (m, 6H), 7.28-7.49 (m, 6H), 7.15 (d, 2H), 3.78-3.90 (m, 1H), 3.52-3.70 (m, 2H), 3.20-3.41 (m, 2H), 2.37 (s, 3H), 2.17-2.29 (m, 1H), 1.31-1.41 (m, 1H), 1.03 (s, 9H), 0.56-0.93 (m, 3 H), −0.01 (q, 1H).

### Description 7: N-[(1S)-1-(hydroxymethyl)-3-buten-1-yl]-4-methylbenzenesulfonamide (D7)

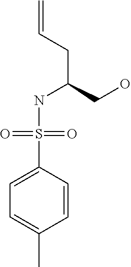

A solution of (2S)-2-amino-4-pentenoic acid (5 g, 43.4 mmol) in THF (200 ml) was cooled down to 0° C. and LiAlH₄ 1 M solution in THF (54.3 ml, 54.3 mmol) was added dropwise. The resulting reaction mixture was allowed to warm-up to room temperature and stirred overnight. The mixture was then cooled down to 0° C. and quenched with a 2 M aqueous NaOH solution. The solid was filtered off and extracted with boiling THF for 1 hour. The combined ethereal extracts were concentrated under reduced pressure and the remaining aqueous mixture extracted with DCM. The combined organic phases were washed with brine, dried (Na₂SO₄) and evaporated under reduced pressure to afford the crude intermediate (2S)-2-amino-4-penten-1-ol (3.82 g) that was used in the next step without any further purification.

A solution of sodium carbonate (6.40 g, 60.4 mmol) in water (35 ml) was left under stirring for 20 minutes at room temperature. (25)-2-amino-4-penten-1-ol (3.82 g) was added, followed by EtOAc (80 ml). After 30 minutes stirring, a solution of p-toluenesulfonyl chloride (5.59 g, 29.3 mmol) in EtOAc (10 ml) and THF (10 ml) was added over 30 minutes. The reaction mixture was stirred at room temperature for 5 hours. Water (30 ml) and EtOAc (100 ml) were then added. The organic phase was separated and the aqueous one extracted with EtOAc (2×50 ml). The combined organic layers were dried (Na₂SO₄), filtered and concentrated under reduced pressure. The residue was purified by flash chromatography on silica gel (Biotage SP, column size 340 g SNAP, from Cy/EtOAc 70:30 to EtOAc 100) to afford the title compound D7 (4.23 g).

UPLC (Acid GEN_QC: rt=0.59 minutes, peak observed: 256 (M+1). C₁₂H₁₇NO₃S requires 255. ¹H-NMR (400 MHz, DMSO-d₆) δ(ppm): 7.68 (d, 2H), 7.48 (d, 1H), 7.37 (d, 2H), 5.48-5.63 (m, 1H), 4.82-4.98 (m, 2H), 4.66 (t, 1H), 3.18-3.27 (m, 1H), 3.00-3.17 (m, 2H), 2.39 (s, 3H), 2.17-2.27 (m, 1H), 1.91-2.03 (m, 1H).

### Description 8: N-[(1S)-1-{[(1,1-dimethylethyl)(diphenyl)silyl]oxy}methyl)-3-buten-1-yl]-4-methylbenzenesulfonamide (D8)

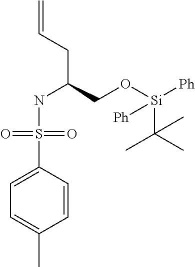

To a solution of N-[(15)-1-(hydroxymethyl)-3-buten-1-yl]-4-methylbenzenesulfonamide D7 (4.23 g) in DMF (35 ml), imidazole (2.98 g, 43.7 mmol) and TBDPSCl (7.49 ml, 29.2 mmol) were added and the resulting reaction mixture was left under stirring overnight at room temperature. The mixture was diluted with water (300 ml) and extracted with EtOAc (5×50 ml). The combined organic phases were dried (Na₂SO₄), filtered and concentrated under reduced pressure to give a yellow oil. The residue was purified by flash chromatography on silica gel (Biotage SP, column size 340 g SNAP, from Cy 100 to Cy/EtOAc 90:10) to afford the title compound D8 (8.07 g) as a crude material which was used in the next step without any further purification.

UPLC (Acid GEN_QC: rt=1.13 minutes, peaks observed: 494 (M+1) and 516 (M+Na). C₂₈H₃₅NO₃SSi requires 493. ¹H-NMR (400 MHz, CDCl₃) δ(ppm): 7.69 (d, 2H), 7.35-7.77 (m, 10H), 7.24 (d, 2H), 5.47-5.63 (m, 1H), 5.01 (bs, 1H), 4.96-5.00 (m, 1H), 4.77 (bd, 1H), 3.57 (dd, 1H), 3.44 (dd, 1H), 3.25-3.37 (m, 1H), 2.43 (s, 3H), 2.30-2.37 (m, 2H), 1.05 (s, 9H).

### Description 9: N-[(1S-1-({[(1,1-dimethylethyl)(diphenyl)silyl]oxy}methyl)-3-buten-1-yl]-4-methyl-N-2-propen-1-ylbenzenesulfonamide (D9)

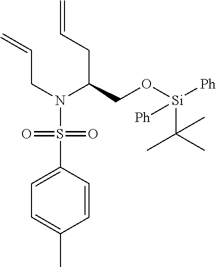

To a solution of N-[(1S)-1-({[(1,1-dimethylethyl)(diphenyl)silyl]oxy}methyl)-3-buten-1-yl]-4-methylbenzenesulfonamide D8 (8.07 g of the crude material obtained in the Description 7) in DMF (30 ml), cesium carbonate (7.46 g, 22.9 mmol) and 3-bromo-1-propene (1.38 g, 11.4 mmol) were added and the mixture was stirred at room temperature overnight. The mixture was diluted with H₂O (300 ml) and extracted with Et₂O (5×50 ml). The combined organic phases were dried (Na₂SO₄), filtered and concentrated under reduced pressure. The residue was purified by flash chromatography on silica gel (Biotage SP, column size 340 g SNAP, from Cy 100 to Cy/EtOAc 90:10) to afford the title compound D9 (7.46 g).

MS: (ES/+) m/z: 534 (M+1) and 556 (M+Na) C₃₁H₃₉NO₃SSi requires 533.

¹H-NMR (400 MHz, CDCl₃) δ(ppm): 7.35-7.79 (m, 12H), 7.20 (d, 2H), 5.72-5.86 (m, 1 H), 5.47-5.62 (m, 1H), 4.88-5.16 (m, 4H), 3.90-4.05 (m, 2H), 3.77-3.88 (m, 1H), 3.59-3.71 (m, 2H), 2.40 (s, 3H), 2.38-2.51 (m, 1H), 2.22-2.33 (m, 1H), 1.04 (s, 9H).

### Description 10: (1R,4S,6R)-4-{[(1,1-dimethylethyl)(diphenyl)silyl]oxy}methyl)-3-azabicyclo[4.1.0]heptane (D10)

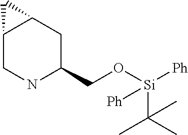

To a solution of (1R,4S,6R)-4-({[(1,1-dimethylethyl)(diphenyl)silyl]oxy}methyl)-3-[(4-methylphenyl)sulfonyl]-3-azabicyclo[4.1.0]heptane D6 (3.6 g) in MeOH (500 ml), under a nitrogen atmosphere, magnesium (9.76 g, 402 mmol) (turnings, previously flame dried) and NH₄Cl (10.37 g, 194 mmol) were subsequently added and the reaction mixture was vigorously stirred at 23° C. After 2 hours further Mg (5 g) was added and the reaction mixture was stirred for other 2.5 hours, then DCM (300 ml) and aqueous NH₄Cl saturated solution (200 ml) were added.

The organic layer was separated and washed with brine (80 ml), filtered through a hydrophobic filter and evaporated under reduced pressure to give a colorless oil which was charged on a SCX (20 g) to afford the title compound D10 (1.81 g).

UPLC (IPQC): rt1=1.00 minutes, peak observed: 365 (M+1) C₂₃H₃₁NOSi requires 364.

¹H NMR (400 MHz, DMSO-d6) δ ppm 0.11-0.19 (m, 1H) 0.50-0.60 (m, 1H) 0.86-1.07 (m, 11H) 1.40-1.56 (m, 2H) 1.63-1.75 (m, 1H) 2.23-2.37 (m, 1H) 2.55-2.65 (m, 1H) 3.43-3.51 (m, 2H) 7.36-7.51 (m, 6H) 7.55-7.67 (m, 4H).

### Description 11: [6-methyl-3-(propyloxy)-2-pyridinyl]methanol (D11)

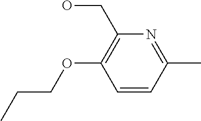

In a 250 ml round-bottomed flask 2-(hydroxymethyl)-6-methyl-3-pyridinol (available from Sigma-Aldrich #144428) (3 g, 21.56 mmol), 1-iodopropane (2.10 ml, 21.56 mmol) and potassium carbonate (14.90 g, 108 mmol) were dissolved in DMF (30 ml) and the mixture left under stirring overnight at room temperature. H₂O and EtOAc were added and the two layers were separated. The aqueous one was back-extracted several times with EtOAc. The combined organic phases were washed with brine/ice, dried (Na₂SO₄), filtered and concentrated under reduced pressure to give a crude material containing the title compound and some residual DMF. The residue was taken-up in water/ice and extracted with EtOAc. The organic phase was dried (Na₂SO₄) and concentrated under reduced pressure to afford the title compound D11 (3.60 g), which was used in the next step without any further purification.

MS: (ES/+) m/z: 182 (M+1) C₁₀H₁₅NO₂ requires 181.

¹H-NMR (400 MHz, CDCl₃) δ(ppm): 6.95-7.09 (m, 2H), 4.73 (s, 2H), 3.94 (t, 2H), 2.50 (s, 3H), 1.75-1.91 (m, 2H), 1.05 (t, 3H).

### Description 12: 6-methyl-3-(propyloxy)-2-pyridinecarboxylic acid (D12)

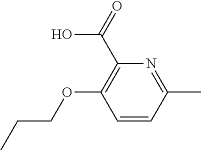

In a 500 ml round-bottomed flask [6-methyl-3-(propyloxy)-2-pyridinyl]methanol D11 (3.50 g) was suspended in water (16 ml) and KMnO₄ (6.10 g, 38.60 mmol) and KOH (1 M aqueous solution, 19 ml, 19 mmol) were added. The mixture was stirred at room temperature for 2 hours. The pH was adjusted to 4 by addition of a 1 M HCl aqueous solution and then MeOH (100 ml) was added. The solid was filtered off, volatiles were removed under reduced pressure and the aqueous phase was extracted twice with DCM. The collected organic layers were washed with brine, dried (Na₂SO₄) and concentrated under reduced pressure to afford the title compound D12 (2 g).

MS: (ES/+) m/z: 196 (M+1) C₁₀H₁₃NO₂ requires 195.

¹H-NMR (400 MHz, DMSO-d₆) δ(ppm): 12.96 (bs, 1H), 7.49 (d, 1H), 7.31 (d, 1H), 3.98 (t, 2H), 2.40 (s, 3H), 1.60-1.80 (m, 2H), 0.96 (t, 3H).

### Description 13: (1R,4S,6R)-4-{[(1,1-dimethylethyl)(diphenyl)silyl]oxy}methyl)-3-{[6-methyl-3-(propyloxy)-2-pyridinyl]carbonyl}-3-azabicyclo[4.1.0]heptane (D13)

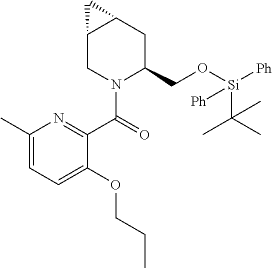

6-methyl-3-(propyloxy)-2-pyridinecarboxylic acid D12 (0.12 g) was dissolved in DMF (1 ml), TBTU (0.197 g, 0.615 mmol) and DIPEA (0.107 ml, 0.615 mmol) were added. The solution was stirred for one hour at room temperature, then (1R,4S,6R)-4-({[(1,1-dimethylethyl)(diphenyl)silyl]oxy}methyl)-3-azabicyclo[4.1.0]heptane D10 (0.164 g) dissolved in DMF (1 ml) was added. The resulting mixture was stirred for an additional hour. The solution was washed with brine and back-extracted with Et₂O. The collected organic phases were dried over Na₂SO₄ and evaporated to give the title compound D13 (0.340 g) as brown oil which was used in the next step without any further purification.

MS: (ES/+) m/z: 543 (M+1) C₃₃H₄₂N₂O₃Si requires 542.

¹H NMR (400 MHz, DMSO-d₆) δ ppm 7.11-7.80 (m, 12H), 4.26-4.55 (m, 2H), 3.04-4.04 (m, 5H), 2.22-2.43 (m, 3H), 1.52-2.22 (m, 4H), 0.78-1.13 (m, 14H), 0.46-0.70 (m, 1H), 0.18-0.35 (m, 1H).

### Description 14: ((1R,4S,6R)-3-{[6-methyl-3-(propyloxy)-2-pyridinyl]carbonyl}-3-azabicyclo[4.1.0]hept-4-yl)methanol (D14)

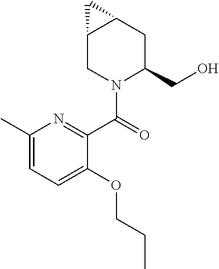

(1R,4S,6R)-4-({[(1,1-dimethylethyl)(diphenyl)silyl]oxy}methyl)-3-{[6-methyl-3-(propyloxy)-2-pyridinyl]carbonyl}-3-azabicyclo[4.1.0]heptane D13 (0.320 g of crude material) was dissolved in pyridine (3 ml) and cooled at 0° C. Hydrogen fluoride-pyridine (0.384 ml, 4.42 mmol) was slowly added and the solution was warmed at room temperature and stirred for 2.5 hours. Water was carefully added (150 ml) and the mixture was extracted with DCM. All the organic phases were combined together, dried over anhydrous Na₂SO₄, filtered and evaporated to dryness to give the crude product which was purified by silica gel chromatography (Biotage SP—column size 10 g SNAP, using Cy/EtOAc 8:2 to Cy/EtOAc 5:5 as eluent) to afford the title compound D14 (0.110 g).

MS: (ES/+) m/z: 305 (M+1) C₁₇H₂₄N₂O₃ requires 304.

¹H NMR (400 MHz, DMSO-d₆) minutes ppm 7.37-7.44 (m, 1H), 7.16-7.23 (m, 1H), 4.77-4.88 (m, 1H), 3.83-4.47 (m, 4H), 2.97-3.60 (m, 3H), 2.34-2.40 (m, 3H), 1.54-1.76 (m, 4H), 0.78-1.10 (m, 5H), 0.45-0.70 (m, 1H), 0.13-0.28 (m, 1H).

### Description 15: [3-(Ethyloxy)-6-methyl-2-pyridinyl]methanol (D15)

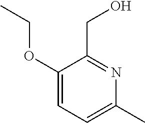

2-(hydroxymethyl)-6-methyl-3-pyridinol (available from Sigma-Aldrich #144428) (1.5 g, 10.78 mmol), K₂CO₃ (7.45 g, 53.9 mmol) and iodoethane (1.724 ml, 21.56 mmol) were dissolved in DMF (15 ml). The mixture was left stirring at room temperature overnight. To the solution were added H₂O and EtOAc. The two layers were separated. The aqueous one was extracted several times with EtOAc. The combined organic layers were washed with brine/ice and dried over Na₂SO₄. The solid was filtered out and the solvent was removed in vacuum to afford the title compound D15 (1.67 g) as a pale yellow solid.

MS: (ES/+) m/z: 182 (M+1) C₉H₁₃NO₂ requires 167.

¹H-NMR (400 MHz, CDCl₃) δ ppm: 6.98-7.06 (m, 2H), 4.72 (s, 2H), 4.47 (bs, 1H), 4.05 (q, 2H), 2.50 (s, 3H), 1.43 (t, 3H).

### Description 16: 3-(Ethyloxy)-6-methyl-2-pyridinecarboxylic acid (D16)

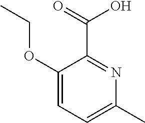

To a solution of [3-(ethyloxy)-6-methyl-2-pyridinyl]methanol D15 (1.67 g) in acetonitrile (50 ml) and phosphate buffer (38 ml) TEMPO (0.218 g, 1.397 mmol) was added at room temperature. After warming to 35° C., a solution of NaClO₂ (4.51 g, 49.9 mmol) in water (10 ml) and a solution of NaClO (18.96 ml, 39.9 mmol) were added simultaneously over 1 hour. After stirring 4 hours at 35° C., water (40 ml) was added to the reaction mixture which was then adjusted to pH 8 by addition of 1 M NaOH. The mixture was poured into ice-cold saturated aqueous sodium thiosulfate solution (100 ml) and stirring was continued for 30 minutes. The pH was adjusted to pH 3 by slow addiction of 1 M HCl and the aqueous phase was extracted with DCM. The combined organic layers were washed with brine, dried over Na₂SO₄ and concentrated to afford the title compound D16 (1.64 g).

MS: (ES/+) m/z: 182 (M+1). C₉H₁₁NO₃ requires 181. ¹H-NMR (400 MHz, DMSO-d₆) δ ppm: 12.90 (bs, 1H), 7.49 (d, 1H), 7.31 (d, 1H), 4.08 (q, 2H), 2.40 (s, 3H), 1.29 (t, 3H).

### Description 17: (1R,4S,6R)-4-{[(1,1-dimethylethyl)(diphenyl)silyl]oxy}methyl)-3-{[3-(ethyloxy)-6-methyl-2-pyridinyl]carbonyl}-3-azabicyclo[4.1.0]heptane (D17)

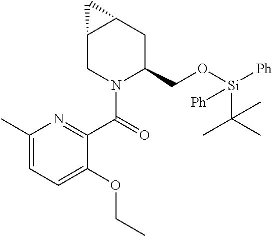

3-(ethyloxy)-6-methyl-2-pyridinecarboxylic acid D16 (0.271 g) was dissolved in DMF (5 ml), TBTU (0.48 g, 1.494 mmol) and DIPEA (0.261 ml, 1.494 mmol) were added. The solution was stirred for one hour at room temperature, then (1R,4S,6R)-4-({[(1,1-dimethylethyl)(diphenyl)silyl]oxy}methyl)-3-azabicyclo[4.1.0]heptane D10 (0.42 g) dissolved in DMF (5 ml) was added. The resulting mixture was stirred for an additional hour. The solution was washed with brine and back-extracted with Et₂O. All the collected organic phases were dried over Na₂SO₄ and evaporated to dryness to give a brown oil which was purified by flash chromatography (silica NH column, size 25 M, eluting with Cy/EtOAc 95:5 to 75:25) to afford the title compound D17 (0.544 g).

MS: (ES/+) m/z: 529 (M+1). C₃₂H₄₀N₂O₃Si requires 528. ¹H NMR (400 MHz, DMSO-d₆) δ ppm 7.14-7.74 (m, 12H), 3.06-4.50 (m, 7H), 2.24-2.41 (m, 3H), 1.57-2.18 (m, 2H), 0.78-1.32 (m, 14H), 0.46-0.70 (m, 1H), 0.23-0.35 (m, 1H).

### Description 18: ((1R,4S,6R)-3-{[3-(ethyloxy)-6-methyl-2-pyridinyl]carbonyl}-3-azabicyclo[4.1.0]hept-4-yl)methanol (D18)

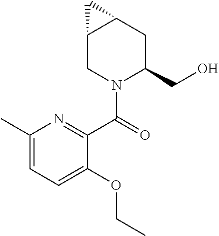

(1R,4S,6R)-4-({[(1,1-dimethylethyl)(diphenyl)silyl]oxy}methyl)-3-{[3-(ethyloxy)-6-methyl-2-pyridinyl]carbonyl}-3-azabicyclo[4.1.0]heptane D17 (0.53 g) was dissolved in pyridine (6 ml) and cooled at 0° C. Hydrogen fluoride-pyridine (0.871 ml, 10.02 mmol) was slowly added and the solution was warmed to room temperature and stirred for 2.5 hours. Water was carefully added and the mixture was extracted with DCM. All the organic phases were combined together, dried over anhydrous Na₂SO₄, filtered and evaporated to dryness to give the crude product which was purified by silica gel chromatography (Biotage SP—column size 25 g SNAP, using Cy/EtOAc 80:20 to Cy/EtOAc 50:50) to afford the title compound D18 (0.32 g).

MS: (ES/+) m/z: 291 (M+1). C₁₆H₂₂N₂O₃ requires 290. ¹H NMR (400 MHz, DMSO-d₆) δ ppm 7.45-7.74 (m, 2H), 2.95-4.48 (m, 7H), 2.33-2.43 (m, 3H), 1.53-2.14 (m, 2H), 1.20-1.42 (m, 3H), 0.75-1.01 (m, 2H), 0.46-0.74 (m, 1H), 0.10-0.37 (m, 1H).

### Description 19: 1,1-dimethylethyl (1R,4S,6R)-4-{[(1,1-dimethylethyl)(diphenyl)silyl]oxy}methyl)-3-azabicyclo[4.1.0]heptane-3-carboxylate (D19)

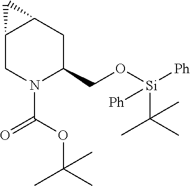

(1R,4S,6R)-4-({[(1,1-dimethylethyl)(diphenyl)silyl]oxy}methyl)-3-azabicyclo[4.1.0]heptane D10 (0.9 g) was dissolved in DCM (50 ml), Boc₂O (0.612 ml, 2.63 mmol) was added, followed by TEA (0.35 ml, 2.51 mmol). The colorless solution was stirred at room temperature for 30 minutes, then it was evaporated at reduced pressure and the residue was dried under high vacuum for 30 minutes. The title compound D19 was obtained as a colourless oil (1.18 g).

UPLC (Basic GEN_QC): rt=1.35 minutes, peak observed: 466 (M+1). C₂₈H₃₉NO₃Si requires 465. ¹H NMR (400 MHz, CDCl₃) δ ppm 7.31-7.76 (m, 10H) 3.15-4.39 (m, 5H) 1.60-2.24 (m, 2H) 1.31-1.51 (m, 9H) 1.06 (s, 9H) 0.49-0.97 (m, 3H) 0.06-0.22 (m, 1 H).

### Description 20:1,1-dimethylethyl (1R,4S,6R)-4-(hydroxymethyl)-3-azabicyclo[4.1.0]heptane-3-carboxylate (D20)

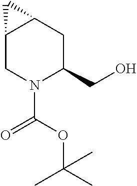

1,1-dimethylethyl (1R,4S,6R)-4-({[(1,1-dimethylethyl)(diphenyl)silyl]oxy}methyl)-3-azabicyclo[4.1.0]heptane-3-carboxylate D19 (1.18 g) was dissolved in THF (8 ml) and TBAF 1 M solution in THF (2.6 ml, 2.60 mmol) was added over 1 minute: the colorless solution turned pale yellow. The mixture was stirred 2.5 hours at room temperature, then new TBAF 1 M solution in THF (1.3 ml, 1.300 mmol) was added over 1 minute and the mixture was stirred again 1 hour at room temperature. New TBAF 1 M solution in THF (0.5 ml, 0.500 mmol) was added and the mixture was stirred again 1 hour at room temperature: complete deprotection. The solvent was removed at reduced pressure and the residue purified by flash chromatography on silica gel (Biotage, Snap-50 g column, EtOAc/Cy from 10:90 to 50:50). After evaporation at reduced pressure of the pure collected fractions the title compound D20 was obtained as colourless oil (0.479 g).

MS: (ES/+) m/z: 172 [(M+1-C(Me)₃+1)]. C₁₂H₂₁NO₃ requires 227. ¹H NMR (400 MHz, DMSO-d₆) δ ppm 4.69 (bs., 1H), 3.71 (m, 2H), 3.34-3.49 (m, 2H), 3.15-3.32 (m, 1H), 1.88 (m, 1H), 1.43-1.62 (m, 1H), 1.31-1.43 (s, 9H), 0.81-0.99 (m, 2H), 0.59 (m, 1H), −0.08 (m, 1H).

### Description 21: 1,1-dimethylethyl (1R,4S,6R)-4-({[5-(trifluoromethyl)-2-pyridinyl]oxy}methyl)-3-azabicyclo[4.1.0]heptane-3-carboxylate (D21)

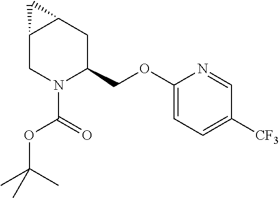

To 1,1-dimethylethyl (1R,4S,6R)-4-(hydroxymethyl)-3-azabicyclo[4.1.0]heptane-3-carboxylate D20 (429 mg), 2-chloro-5-(trifluoromethyl)pyridine (380 mg, 2.093 mmol) and K₂CO₃ (300 mg, 2.171 mmol) DMF (7 ml) was added and the mixture was stirred at 110° C. overnight. New 2-chloro-5-(trifluoromethyl)pyridine (380 mg, 2.093 mmol) was added, followed by NaH (226 mg, 5.66 mmol). The mixture was stirred at room temperature for 30 minutes. The reaction was quenched by a slow and careful addition of NaHCO₃ saturated solution (5 ml) (gas evolution), then it was partitioned between NaHCO₃ saturated solution and DCM; aqueous layer extracted with DCM. The organic phases were joined, washed with brine, dried over Na₂SO₄ and evaporated at reduced pressure. The so obtained yellow/orange oily residue was purified by flash chromatography on silica gel (Biotage, Snap-100 g column, from Cy to EtOAc/Cy 20:80). The title compound D21 was obtained as colourless oil (506 mg).

UPLC (Basic GEN_QC: rt=0.62 minutes and rt=1.1 minutes (rotamers present), peak observed: 372 (M+1). C₁₈H₂₃F₃N₂O₃ requires 373. ¹H NMR (400 MHz, CDCl₃) δ ppm 8.44 (s, 1H), 7.70-7.87 (m, 1H), 6.82 (m, 1H), 4.45 (m, 2H), 3.91 (m, 1H), 3.43 (m, 1H), 2.07 (m, 1H), 2.02 (m, 1H), 1.80 (m, 1H), 1.32-1.53 (m, 9H), 0.85-1.11 (m, 2H), 0.72 (m, 1H), 0.01-0.19 (m, 1H).

### Description 22: (1R,4S,6R)-4-({[5-(trifluoromethyl)-2-pyridinyl]oxy}methyl)-3-azabicyclo[4.1.0]heptane (D22)

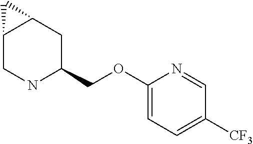

1,1-dimethylethyl (1R,4S,6R)-4-({[5-(trifluoromethyl)-2-pyridinyl]oxy}methyl)-3-azabicyclo[4.1.0]heptane-3-carboxylate D21 (547 mg) was dissolved in DCM (20 ml); TFA (2 ml, 26.0 mmol) dissolved in DCM (5 ml) was added over 1 minute. The mixture was stirred at room temperature for 1 hour, then it was purified by SCX-10 g column obtaining the crude amine, which was then purified by flash chromatography on silica gel (Biotage, Snap-100 g column, DCM/MeOH 95:5). The title compound D22 was obtained as pale yellow solid (348 mg).

UPLC (Basic GEN_QC: rt=0.62 minutes and rt=0.83 minutes (rotamers present), peak observed: 273 (M+1). C₁₃H₁₅F₃N₂O requires 272. ¹H NMR (500 MHz, DMSO-d₆) δ ppm 8.52-8.59 (m, 1H), 8.05 (dd, 1H), 7.00 (d, 1H), 4.13-4.24 (m, 2H), 3.28-3.32 (m, 1H), 2.63-2.68 (m, 1H), 2.56-2.63 (m, 1H), 2.14 (m, 1H), 1.74-1.83 (m, 1H), 1.49-1.62 (m, 1H), 0.94-1.05 (m, 2H), 0.55-0.64 (m, 1H), 0.17-0.26 (m, 1H).

### Description 23: 2-methylfuro[3,4-b]pyridine-5,7-dione (D23)

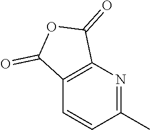

In a 100 ml round-bottomed flask 6-methyl-2,3-pyridinedicarboxylic acid (10 g, 55.2 mmol) and acetic anhydride (26 ml, 276 mmol) were added and heated at 100° C. under nitrogen for 5 hours. After this time the volatiles were removed under vacuum to give the title compound D23 (8.2 g) as a slightly brown solid.

¹H NMR (400 MHz, DMSO-d₆) δ ppm 8.41 (d, 1H), 7.82 (d, 1H), 2.73 (s, 3H).

### Description 24: 6-methyl-2-[(methyloxy)carbonyl]-3-pyridinecarboxylic acid (D24)

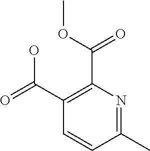

2-methylfuro[3,4-b]pyridine-5,7-dione D23 (3 g) was added portionwise over 5 minutes to stirred MeOH (20 ml) at 0° C. The mixture was stirred at 0° C. for 30 minutes then at room temperature for other 2.5 hours. The solution was evaporated at reduced pressure and the residue recrystallized from toluene (50 ml). The solid was filtered and dried under high vacuum for 30 minutes, obtaining a first batch of the title compound D24 (1.16 g) as pale brown solid. From the toluene solution new solid precipitated: this solid was filtered and dried under high vacuum for 30 minutes, obtaining a second batch of the title compound D24 (352 mg) as pale yellow solid. The toluene solution was then evaporated at reduced pressure and the residue recrystallized again from toluene (25 ml). The solid was filtered and dried under high vacuum for 30 minutes, obtaining a third batch of the title compound D24 (615 mg) as pale yellow solid.

UPLC (Basic GEN_QC): rt=0.23 minutes, peak observed: 195 (M+1). C₉H₉NO₄ requires 196. ¹H NMR (400 MHz, DMSO-d₆) δ ppm 13.61 (br. s., 1H), 8.09-8.31 (m, 1H), 7.51 (m, 1H), 3.82 (s, 3H), 2.55 (s, 3H).

### Description 25: methyl 3-{[(1,1-dimethylethyl)oxy]carbonyl}amino)-6-methyl-2-pyridinecarboxylate (D25)

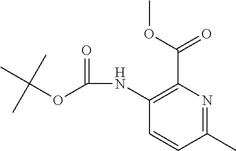

6-methyl-2-[(methyloxy)carbonyl]-3-pyridinecarboxylic acid D24 (1.15 g) was suspended in toluene (40 ml) and DIPEA (1.25 ml, 7.16 mmol) was added, causing the complete dissolution of the solid. This mixture was stirred 10 minutes at room temperature, then diphenyl azidophosphate (1.35 ml, 6.26 mmol) was added in one portion and the mixture was stirred at reflux for 1 hour. The solution was cooled at room temperature and t-BuOH (2.5 ml, 26 mmol) was added in one portion.

The mixture was then stirred at 70° C. for 1 hour and then cooled at room temperature, Et₂O (50 ml) was added and the resulting solution washed with NaHCO₃ saturated solution. The water phases were joined together and back-extracted with Et₂O. The two organic solutions were joined together, dried over Na₂SO₄ and evaporated at reduced pressure, obtaining the crude target material as pale yellow oil. This material was purified by flash chromatography on silica gel (Biotage, EtOAc/Cy from 10:90 to 70:30; Snap-100 g column). The title compound D25 (1.315 g) was obtained as white solid.

UPLC (Basic GEN_QC: rt=0.68 minutes, peak observed: 267 (M+1). C₁₃H₁₈N₂O₄ requires 266. ¹H NMR (400 MHz, CDCl₃) δ ppm 10.13 (bs., 1H), 8.77 (d, 1H), 7.34 (d, 1 H), 4.03 (s, 3H), 2.59 (s, 3H), 1.53-1.56 (m, 9H).

### Description 26: methyl 3-amino-6-methyl-2-pyridinecarboxylate (D26)

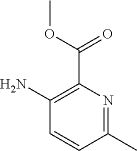

Methyl 3-({[(1,1-dimethylethyl)oxy]carbonyl}amino)-6-methyl-2-pyridinecarboxylate D25 (1.3 g) was dissolved in DCM (80 ml) and the mixture stirred at 0° C. A solution of TFA (5 ml, 64.9 mmol) in DCM (10 ml) was dropped into the cold mixture over 3 minutes. The resulting solution was left under stirring at 0° C. for 30 minutes, then the mixture was left still at room temperature overnight. TFA (4 ml, 51.9 mmol) dissolved in DCM (10 ml) was added over 3 minutes and the mixture stirred again at room temperature for 5 hours. The solution was loaded onto an SCX-25 g column to afford the title compound D26 (770 mg) as a white solid.

UPLC (Basic GEN_QC: rt=0.44 minutes, peak observed: 167 (M+1). C₈H₁₀N₂O₂ requires 166. ¹H NMR (400 MHz, CDCl₃) δ ppm 7.14 (d, 1H), 7.01 (d, 1H), 3.99 (s, 3H), 2.52 (s, 3 H).

### Description 27: methyl 3-iodo-6-methyl-2-pyridinecarboxylate (D27)

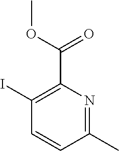

HCl 6 M solution in water (4.5 ml, 27.0 mmol) was added to methyl 3-amino-6-methyl-2-pyridinecarboxylate D26 (768 mg) and the resulting pale yellow mixture was sequentially diluted with water (4×5 ml) and chilled at 0° C. (internal temperature).

A solution of sodium nitrite (480 mg, 6.96 mmol) in water (2 ml) was dropped into the mixture over 1 minute. After this addition the mixture was stirred at 0° C. for 30 minutes, then a solution of KI (1.69 g, 10.18 mmol) in water (2 ml) was added over 1 minute, causing the formation of a dark violet crust (moderate gas evolution). The mixture was left under stirring for 1 hour: during this period the temperature passed from 0° C. to +5° C. EtOAc (50 ml) was then added to the stirred mixture, causing the dissolution of the dark solid. Water (50 ml) and EtOAc (50 ml) were added and the whole mixture was poured into a separator funnel. After the separation of the two phases, the water phase was extracted with EtOAc. All the organic phases were joined together and washed with NaHCO₃ saturated solution; the acidic water phase was neutralized by the addition of NaHCO₃ saturated solution and the resulting mixture extracted with EtOAc. All the organic phases were joined together, dried over Na₂SO₄ and evaporated at reduced pressure, obtaining the crude target material as dark brown/violet oil. This material was purified by silica gel chromatography (Biotage SP4 Snap-100 g column, EtOAc/Cy from 10:90 to 30:70). The title compound D27 (1.1 g) was obtained as a pale brown solid.

UPLC (Basic GEN_QC): rt=0.68 minutes, peak observed: 278 (M+1). C₈H₈INO₂ requires 277. ¹H NMR (400 MHz, CDCl₃) δ ppm 8.12 (d, 1H), 7.01 (d, 1H), 4.01 (s, 3H), 2.58 (s, 3 H).

### Description 28: methyl 6-methyl-3-(2-pyrimidinyl)-2-pyridinecarboxylate (D28)

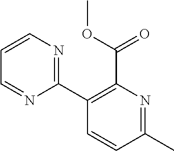

To a suspension of methyl 3-iodo-6-methyl-2-pyridinecarboxylate D27 (300 mg), CsF (329 mg, 2.166 mmol) and Pd(Ph₃P)₄ (50.0 mg, 0.043 mmol) in DMF (10 ml) stirred under nitrogen at room temperature, was added 2-(tributylstannanyl)pyrimidine (480 mg, 1.299 mmol). The reaction mixture was stirred at 130° C. for 30 minutes in a microwave Personal Chemistry. The reaction mixture was partitioned between EtOAc and aqueous NaHCO₃ saturated solution; the combined organic phases were dried to give the crude product, which was purified by silica gel chromatography (SNAP KP—NH 55 g; Cy/EtOAc from 100:0 to 70:30). Collected fractions were evaporated to obtain the title compound D28 (101 mg) as white solid.

UPLC (Basic GEN_QC): rt=0.56 minutes, peak observed: 230 (M+1). C₁₂H₁₁N₃O₂ requires 229. ¹H NMR (400 MHz, DMSO-d₆) δ ppm 8.92 (d, 2H), 8.49 (d, 1H), 7.44-7.63 (m, 2 H), 3.75 (s, 3H), 2.57 (s, 3H).

### Description 29: 6-methyl-3-(2-pyrimidinyl)-2-pyridinecarboxylic acid lithium salt (D29)

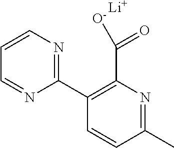

To a solution of methyl 6-methyl-3-(2-pyrimidinyl)-2-pyridinecarboxylate D28 (100 mg) in MeOH (4.5 ml) and water (1.1 ml) was added LiOH (13.58 mg, 0.567 mmol) and the resulting mixture was submitted to microwave irradiation at 60° C. for 85 minutes. After this time the solvents were removed under reduced pressure to give the title compound D29 (100 mg) as a white solid.

C₁₁H₈N₃O₂.Li⁺ requires 221. ¹H NMR (400 MHz, DMSO-d₆) δ ppm 8.78 (m, 2H), 7.86 (m, 1H), 7.37 (m, 1H), 7.24 (m, 1H), 2.50 (s, 3H).

### Description 30: methyl 6-methyl-3-(4-methyl-1,3-thiazol-2-yl)-2-pyridinecarboxylate (D30)

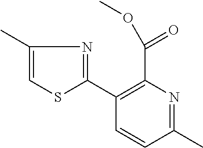

4-methyl-2-(tributylstannanyl)-1,3-thiazole (150 mg, 0.386 mmol) was dissolved in 1,4-Dioxane (2.5 ml). To the stirred solution methyl 3-iodo-6-methyl-2-pyridinecarboxylate D27 (100 mg) was added, followed by Pd(Ph₃P)₄ (41.7 mg, 0.036 mmol).

The resulting orange solution was heated into a microwave reactor at 120° C. for 30 minutes: complete conversion. The mixture was loaded onto an SCX-5 g column. After evaporation of the ammoniacal solution it was obtained the crude target material as colorless oil, which was then purified by flash chromatography on silica gel (Biotage SNAP-10 g silica gel column, EtOAc/Cy 25:75). It was obtained the title compound D30 (74 mg) as white solid.

MS: (ES/+) m/z: 249 (M+1). C₁₂H₁₂N₂O₂S requires 248.

¹H NMR (400 MHz, CDCl₃) δ ppm 7.97 (d, 1H), 7.33 (d, 1H), 6.98 (s, 1H), 3.94 (s, 3H), 2.66 (s, 3H), 2.50 (s, 3H).

### Description 31: 6-methyl-3-(4-methyl-1,3-thiazol-2-yl)-2-pyridinecarboxylate lithium salt (D31)

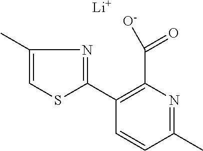

Methyl 6-methyl-3-(4-methyl-1,3-thiazol-2-yl)-2-pyridinecarboxylate D30 (73 mg) was dissolved in EtOH (1 ml) into a capped vial, then a solution of LiOH (8.5 mg, 0.355 mmol) in water (0.5 ml) was added in one portion. The mixture was then stirred at room temperature for 3 hours. The solvent was evaporated at reduced pressure, obtaining the title compound D31 as pale yellow solid (73 mg).

C₁₁H₉N₂O₂S Li⁺ requires 241. ¹H NMR (400 MHz, DMSO-d₆) δ ppm 8.04 (d, 1H), 7.22 (d, 1H), 7.08 (d, 1H), 2.39 (s, 3H), 2.42 (s, 3H).

### Description 32: methyl 6-methyl-3-(2H-1,2,3-triazol-2-yl)-2-pyridinecarboxylate (D32)

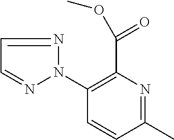

DMF (1.5 ml) was added to a mixture of methyl 3-iodo-6-methyl-2-pyridinecarboxylate D27 (100 mg), 1H-1,2,3-triazole (49.9 mg, 0.722 mmol), (1R,2R)—N,N′-dimethyl-1,2-cyclohexanediamine (10.27 mg, 0.072 mmol), CuI (3.44 mg, 0.018 mmol) and Cs₂CO₃ (235 mg, 0.722 mmol) in a microwave vial. The mixture was degassed via three vacuum/nitrogen cycles then irradiated in a single mode microwave reactor to 120° C. for 20 minutes. The mixture was irradiated in a single mode microwave reactor to 120° C. for a further 40 minutes. The reaction mixture was cooled and filtered washing the solids with EtOAc. The solids were dissolved in pH=3 buffer solution (5 ml); UPLC check of this aqueous solution showed it contained a considerable quantity of 6-methyl-3-(2H-1,2,3-triazol-2-yl)-2-pyridinecarboxylic acid. The aqueous phase was extracted repeatedly with DCM; the combined DCM extracts were diluted with MeOH (50 ml) and treated with TMS-diazomethane. The volatiles were evaporated to give a yellow residue that was purified by flash chromatography on silica gel (Biotage, SNAP 10 g column, 10%-50% EtOAc/cyclohexane) to give the title compound D32 (38 mg) as a white solid.

MS: (ES/+) m/z: 219 (M+1). C₁₀H₁₀N₄O₂ requires 218.

¹H NMR (400 MHz, CDCl₃) δ ppm 8.20 (d, 1H), 7.87 (s, 2H), 7.44 (d, 1H), 3.94 (s, 3H), 2.71 (s, 3H).

### Description 33: 6-methyl-3-(2H-1,2,3-triazol-2-yl)-2-pyridinecarboxylic acid (D33)

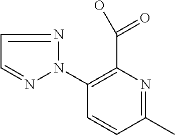

A solution of methyl 6-methyl-3-(2H-1,2,3-triazol-2-yl)-2-pyridinecarboxylate D32 (36 mg) and LiOH (5.93 mg, 0.247 mmol) in THF/water (2:1, 3 ml) was stirred overnight. The mixture was evaporated under reduced pressure; the residue was taken up in water (2 ml) and neutralised with 1 M HCl water solution and then loaded onto a pre-conditioned C18 column (5 g). The column was eluted with water and then MeOH. The methanol fractions were evaporated under reduced pressure to give the title compound D33 (34 mg) as a white solid.

MS: (ES/+) m/z: 205 (M+1) C₉H₈N₄O₂ requires 204.

¹H NMR (400 MHz, MeOD) δ ppm 8.24 (d, 1H), 7.99 (s, 2H), 7.61 (d, 1H), 2.67 (s, 3H).

### Description D34: methyl 6-methyl-3-(4-methyl-2H-1,2,3-triazol-2-yl)-2-pyridinecarboxylate (D34)

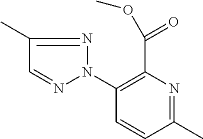

DMF (1.5 ml) was added to a mixture of methyl 3-iodo-6-methyl-2-pyridinecarboxylate D27 (200 mg), 4-methyl-1H-1,2,3-triazole (120 mg, 1.444 mmol), (1R,2R)—N,N′-dimethyl-1,2-cyclohexanediamine (20.54 mg, 0.144 mmol), copper(I)trifluoromethanesulfonate benzene complex (18.17 mg, 0.036 mmol) and cesium carbonate (470 mg, 1.444 mmol) in a screw-topped vial. The mixture was degassed via 3 vacuum/nitrogen cycles and heated with shaking in a PLS reaction station to 120° C. for 5 hours. The reaction mixture was evaporated to dryness under reduced pressure. The residue was dissolved in water/MeOH (1:1, 3 ml) and acidified to pH=2 by addition of 2 M HCl solution. The resulting mixture was evaporated to dryness under reduced pressure then the residue was triturated with DCM/MeOH (3:1, 5 ml). The mixture was filtered washing with more DCM/MeOH (3:1, 5 ml). The filtrate was treated with TMS-diazomethane solution 2 M in hexane (4 ml, 8 mmol) to re-esterify the acid. The reaction mixture was evaporated under reduced pressure and the residue was purified via Biotage (20%-50% EtOAc/cyclohexane; SNAP 25 silica column) to give the title compound D34 (121 mg) as colourless solid.

UPLC (Acid QC_POS_{—}50-800): rt=0.59 minutes, peak observed: 233 (M+1) C₁₁H₁₂N₄O₂ requires 232. ¹H NMR (400 MHz, CDCl₃) δ ppm 8.15 (d, 1H), 7.59 (s, 1H), 7.37 (d, 1H), 3.92 (s, 3H), 2.66 (s, 3H), 2.40 (s, 3H).

### Description D35: 6-methyl-3-(4-methyl-2H-1,2,3-triazol-2-yl)-2-pyridinecarboxylic acid (D35)

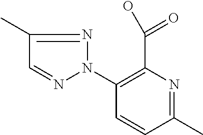

A solution of methyl 6-methyl-3-(4-methyl-2H-1,2,3-triazol-2-yl)-2-pyridinecarboxylate D34 (120 mg) and lithium hydroxide (18.56 mg, 0.775 mmol) in THF/water (2:1, 4.5 ml) was stirred for 2 hours. The mixture was stirred for another 2 hours then evaporated under reduced pressure; the residue was taken up in water (3 ml) and the pH was adjusted to pH=2 with 1 M HCl solution. The mixture was loaded onto a pre-conditioned C18 column (10 g, eluted with water and then MeOH). The methanol fractions were evaporated under reduced pressure to give the title compound D35 (109 mg) as white solid.

UPLC (Acid QC_POS_{—}50-800): rt=0.59 minutes, peak observed: 219 (M+1) C₁₀H₁₀N₄O₂ requires 218.

¹H NMR (400 MHz, CDCl₃) δ ppm 8.00 (d, 1H), 7.65 (s, 1H), 7.52 (d, 1H), 2.71 (s, 3H), 2.43 (s, 3H).

### Description D36: methyl 6-methyl-3-(1H-pyrazol-1-yl)-2-pyridinecarboxylate (D36)

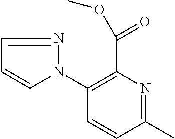

DMF (1.5 ml) was added to a mixture of methyl 3-iodo-6-methyl-2-pyridinecarboxylate D27 (200 mg), 1H-pyrazole (98 mg, 1.444 mmol), (1R,2R)—N,N′-dimethyl-1,2-cyclohexanediamine (20.54 mg, 0.144 mmol), bis(copper(I)trifluoromethanesulfonate), benzene complex (18.17 mg, 0.036 mmol) and cesium carbonate (470 mg, 1.444 mmol) in a screw-topped vial. The mixture was degassed via 3 vacuum/nitrogen cycles and heated with shaking in a PLS reaction station to 120° C. for 1 hour. The reaction mixture was evaporated to dryness under reduced pressure. The residue was dissolved in water/MeOH (1:1, 3 ml) and acidified to pH=2 by addition of 4 M HCl solution. The resulting mixture was evaporated to dryness under reduced pressure then the residue was triturated with DCM/MeOH (3:1, 20 ml). The mixture was filtered washing with more DCM/MeOH (3:1, 5 ml). The filtrate was treated with TMS-diazomethane solution (2 M in hexanes, 2 ml, 4 mmol) to re-esterify the acid. The reaction mixture was evaporated under reduced pressure and the residue was purified twice via Biotage (20%-50% EtOAc/cyclohexane; SNAP 10 silica column and then 1% EtOAc/DCM isocratic SNAP 11 NH column) to give the title compound D36 (107 mg) as colourless gum.

UPLC (Basic QC_{—}50-800_POS): rt=0.51 minutes, peak observed: 218 (M+1) C₁₁H₁₁N₃O₂ requires 217.

¹H NMR (400 MHz, CDCl₃) δ ppm 7.63-7.86 (m, 3H), 7.39 (m, 1H), 6.48 (m, 1H), 3.85 (s, 3H), 2.68 (s, 3H).

### Description D37: 6-methyl-3-(1H-pyrazol-1-yl)-2-pyridinecarboxylic acid (D37)

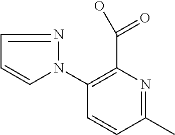

A solution of methyl 6-methyl-3-(1H-pyrazol-1-yl)-2-pyridinecarboxylate D36 (106 mg) and LiOH (17.53 mg, 0.732 mmol) in THF/water (2:1, 6 ml) was stirred overnight. The mixture was evaporated under reduced pressure; the residue was taken up in water (2 ml) and the pH was adjusted to pH=2 with 1 M HCl solution. The mixture was loaded onto a pre-conditioned C18 column (5 g, eluted with water and then MeOH). The methanol fractions were evaporated under reduced pressure to give the title compound D37 (98 mg) as white solid.

UPLC (Basic QC_{—}50-800 POS): rt=0.30 minutes, peak observed: 160 [(M-CO₂)+1] C₁₀H₉N₃O₂ requires 203.

¹H NMR (400 MHz, MeOD) δ ppm 7.77-8.03 (m, 2H), 7.74 (m, 1H), 7.58 (m, 1H), 6.55 (m, 1H), 2.66 (s, 3H).

### Description 38: 2-{[(1,1-Dimethylethyl)(dimethyl)silyl]oxy}methyl)-6-methyl-3-pyridinol (D38)

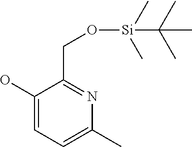

Imidazole (7.71 g, 113 mmol) and tert-butyldimethylsilyl chloride (6.82 g, 45.3 mmol) were added to a solution of 2-(hydroxymethyl)-6-methyl-3-pyridinol (5.25 g, 37.7 mmol) in anhydrous DMF (150 ml) with stirring at room temperature. The mixture was then stirred at 60° C. under nitrogen overnight. The mixture was diluted with DCM and washed with NH₄Cl saturated solution and brine. The organic layer was evaporated and dried over Na₂SO₄. The residual material was purified by flash chromatography on silica gel (SP1, 40 M column, with Cy/EtOAc: from Cy 100 to Cy/EtOAc 90:10 in 10 CV and then Cy/EtOAc 90:10 elution) to afford the title compound D38 (5.52 g) as a white solid.

MS: (ES/+) m/z: 254 (M+1) C₁₃H₂₃NO₂Si requires 253. ¹H NMR (400 MHz, DMSO-d₆) δ ppm: 9.5 (s, 1H), 7.03-7.06 (m, 1H), 6.95-6.98 (m, 1H), 4.67 (s, 2H) 2.33 (s, 3H), 0.87-0.85 (m, 9H), 0.06-0.04 (m, 6H).

### Description 39: 2-({[(1,1-Dimethylethyl)(dimethyl)silyl]oxy}methyl)-6-methyl-3-pyridinyl trifluoromethanesulfonate (D39)

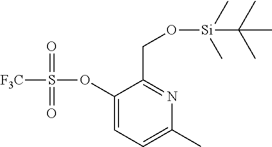

To a solution of 2-({[(1,1-dimethylethyl)(dimethyl)silyl]oxy}methyl)-6-methyl-3-pyridinol D38 (0.52 g) in anhydrous DCM (10 ml) was added DIPEA (1.075 ml, 6.16 mmol) dropwise with stirring. The mixture was then cooled to 0° C. and triflic anhydride (0.52 ml, 3.08 mmol) was added dropwise with stirring. The solution was allowed to warm up to room temperature and stirred under nitrogen for 4 hours. The solution was diluted with DCM and washed with water. The organic layer was then dried over Na₂SO₄ and evaporated. The residual brown oil was purified by flash chromatography on silica gel (Companion, 120 g cartridge, with Cy/EtOAc: from Cy 100 to Cy/EtOAc 80:20 elution) to afford the title compound D39 (0.62 g) as a yellow oil.

MS: (ES/+) m/z: 386 (M+1) C₁₄H₂₂F₃NO₄SSi requires 385.

¹H NMR (400 MHz, DMSO-d₆) δ ppm: 7.85-7.78 (d, 1H), 7.45-7.43 (d, 1H), 4.79 (s, 2H) 2.53-2.49 (m, 3H), 0.87-0.85 (m, 9H), 0.06-0.04 (m, 6H).

### Description 40: 2-{[(1,1-Dimethylethyl)(dimethyl)silyl]oxy}methyl)-6-methyl-3-phenylpyridine (D40)

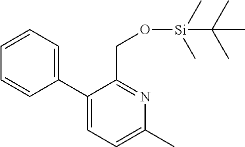

Nitrogen was passed through a suspension of 2-({[(1,1-dimethylethyl)(dimethyl)silyl]oxy}methyl)-6-methyl-3-pyridinyl trifluoromethanesulfonate D39 (0.200 g), phenyl boronic acid (0.127 g, 1.038 mmol) and anhydrous K₂CO₃ (0.108 g, 0.778 mmol) in toluene (5 ml) for 15 minutes. Pd(Ph₃P)₄ (0.060 g, 0.052 mmol) was added and the mixture was heated at 85-90° C. for 5 hours. The reaction mixture was cooled to 25° C., diluted with EtOAc and washed sequentially with saturated NaHCO₃ aqueous solution, NH₄Cl, water and brine. The organic phase was concentrated and the residue was purified by flash chromatography on silica gel (Companion, 80 g cartridge, with Cy/EtOAc from Cy 100 to Cy/EtOAc 95:5 in 10 CV, Cy/EtOAc 95:5 3 CV, from Cy/EtOAc 95:5 to Cy/EtOAc 80:20 in 7 CV elution) to afford the title compound D40 (0.114 g) as a yellow oil.

MS: (ES/+) m/z: 314 (M+1) C₁₉H₂₇NOSi requires 313.

¹H NMR (400 MHz, DMSO-d₆) δ ppm 7.59 (d, 1H), 7.35-7.48 (m, 5H), 7.28 (d, 1H), 4.61 (s, 2H), 2.53-2.49 (m, 3H), 0.79-0.93 (m, 9H), −0.06-0.04 (m, 6H).

### Description 41: (6-Methyl-3-phenyl-2-pyridinyl)methanol (D41)

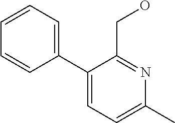

A solution of 2-({[(1,1-dimethylethyl)(dimethyl)silyl]oxy}methyl)-6-methyl-3-phenylpyridine D40 (0.99 g) in TBAF 1.0 M solution in THF (10 ml, 10.00 mmol) was stirred at room temperature for 30 minutes. The solvent was removed in vacuo and the residue was taken up in water (15 ml). The resulting solution was washed with DCM. The combined organic layers were dried (Na₂SO₄) and evaporated. The residual yellow oil was purified by flash chromatography on silica gel (Companion, 120 g cartridge with Cy/EtOAc from Cy 100 to Cy 70:30 elution) to afford the title compound D41 (0.53 g) as a white solid.

HPLC (walk up): rt=2.31 minutes, C₁₃H₁₃NO requires 199.

¹H NMR (400 MHz, DMSO-d₆) δ ppm 7.60 (d, 1H), 7.34-7.51 (m, 5H), 7.27 (d, 1H), 5.12 (m, 1H), 4.33-4.45 (m, 2H), 2.54-2.49 (m, 3H).

### Description 42: 6-Methyl-3-phenyl-2-pyridinecarboxylic acid (D42)

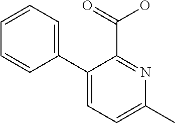

To a solution of (6-methyl-3-phenyl-2-pyridinyl)methanol D41 (0.2 g) in water (3 ml) was added dropwise a solution of KMnO₄ (0.206 g, 1.305 mmol) in water (7 ml) at 5-10° C. with vigorous stirring, then the reaction mixture was stirred at room temperature overnight and then filtered through a plugh of celite (MnO₂ was removed). The filtrate was concentrated under reduced pressure. The unreacted substance was removed by extraction with DCM. The pH of the aqueous layer was adjusted to pH=5.5 with 2 N HCl and the product was extracted with DCM. The organic layers were collected, dried over Na₂SO₄ and evaporated to afford the title compound D42 (0.056 g) as a white solid.

UPLC (Basic GEN_QC: rt=0.64 minutes, peak observed: 214 (M+1) C₁₃H₁₁NO₂ requires 213.

¹H NMR (400 MHz, DMSO-d₆) δ ppm 13.23 (br. s., 1H), 7.78 (d, 1H), 7.50-7.35 (m, 6 H), 2.53 (s, 3H).

### Description 43: (1R,4S,6R)-4-{[(1,1-dimethylethyl)(diphenyl)silyl]oxy}methyl)-3-[(6-methyl-3-phenyl-2-pyridinyl)carbonyl]-3-azabicyclo[4.1.0]heptane (D43)

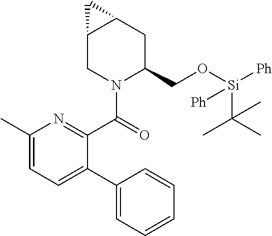

To a solution of (1R,4S,6R)-({[(1,1-dimethylethyl)(diphenyl)silyl]oxy}methyl)-3-azabicyclo[4.1.0]heptane D10 (439 mg) in DMF (5 ml) at 20° C., TBTU (386 mg, 1.201 mmol), 6-methyl-3-phenyl-2-pyridinecarboxylic acid D42 (300 mg) and DIPEA (0.630 ml, 3.60 mmol) were added. Reaction was stirred overnight, then NaHCO₃ saturated solution was added and the mixture extracted twice with DCM. Solvent was removed under reduced pressure to give the title compound D43 (710 mg) as orange oil which was used without further purification in following step.

UPLC (Acid GEN_QC: rt1=1.22 minutes and rt2=1.26 minutes (rotamers present), peak observed: 561 (M+1) C₃₆H₄₀N₂O₂Si requires 560.

### Description 44: {(1R,4S,6R)-3-[(6-methyl-3-phenyl-2-pyridinyl)carbonyl]-3-azabicyclo[4.1.0]hept-4-yl}methanol (D44)

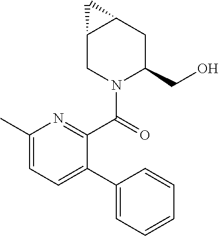

To a solution of (1R,4S,6R)-4-({[(1,1-dimethylethyl)(diphenyl)silyl]oxy}methyl)-3-[(6-methyl-3-phenyl-2-pyridinyl)carbonyl]-3-azabicyclo[4.1.0]heptane D43 (710 mg) in THF (2 ml) at 20° C., TBAF (1.646 ml, 1.646 mmol) was added. After 3 hours further TBAF (0.5 ml) was added. After further 2 hours NaHCO₃ saturated solution was added and the mixture extracted twice with DCM. Solvent was removed to give a crude that was added to a silica gel column and was eluted with DCM/MeOH 0 to 20% to give title compound D44 (250 mg) as colourless oil.

UPLC (Acid GEN_QC): rt1=0.63 minutes and rt2=0.65 minutes (rotamers present), peak observed: 323 (M+1) C₂₀H₂₂N₂O₂ requires 322.

¹H NMR (400 MHz, CDCl₃-d) d ppm 7.60-7.74 (m, 1H) 7.35-7.55 (m, 5H) 7.19-7.26 (m, 1H) 4.56 (d, 1H) 3.28-3.92 (m, 4H) 2.59 (s, 3H) 1.53-1.76 (m, 2H) 0.55-1.14 (m, 3H) −0.23-−0.15 (m, 1H).

### Description 45: (1R,4S,6R)-4-{[(1,1-dimethylethyl)(diphenyl)silyl]oxy}methyl)-3-{[6-methyl-3-(2-pyrimidinyl)-2-pyridinyl]carbonyl}-3-azabicyclo[4.1.0]heptane (D45)

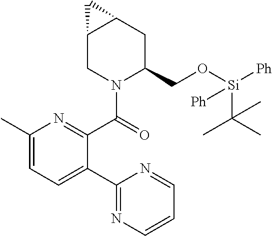

(1R,4S,6R)-4-({[(1,1-dimethylethyl)(diphenyl)silyl]oxy}methyl)-3-azabicyclo[4.1.0]heptane D10 (850 mg), 6-methyl-3-(2-pyrimidinyl)-2-pyridinecarboxylic acid D59 (650 mg) T₃P (1480 mg, 2.325 mmol) and DIPEA (0.406 ml, 2.325 mmol) were collected in DCM (20 ml) and reacted at room temperature for 2 hours then monitored. The reaction was not completed. It was then stirred at 45° C. (external temperature) for further 2 hours. It was then taken up with DCM (200 ml) and washed with water, dried over Na₂SO₄, filtered and concentrated. The crude was then purified with Biotage (SP 1 over a 100 g SNAP KP—NH column, eluting with a gradient of Cy/EtOAc), to afford the title compound D45 as yellow oil (710 mg).

UPLC (Acid GEN_QC_SS): rt1=1.23 minutes and rt2=1.25 minutes (rotamers present), peak observed: 563 (M+1) C₃₄H₃₈N₄O₂Si requires 562.

### Description 46: ((1R,4S,6R)-3-{[6-methyl-3-(2-pyrimidinyl)-2-pyridinyl]carbonyl}-3-azabicyclo[4.1.0]hept-4-yl)methanol (D46)

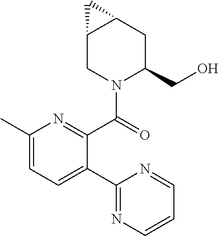

(1R,4S,6R)-4-({[(1,1-dimethylethyl)(diphenyl)silyl]oxy}methyl)-3-{[6-methyl-3-(2-pyrimidinyl)-2-pyridinyl]carbonyl}-3-azabicyclo[4.1.0]heptane D45 (810 mg) was dissolved in THF (20 ml) and reacted with TBAF (2.88 ml, 2.88 mmol) at room temperature for 4 hours. The reaction was concentrated under vacuum and the resulting oil was purified with Biotage (Sp 1, over a 150 Analogix Silica column, eluting with a gradient of DCM and MeOH) to afford the title compound D46 (430 mg) as yellow oil.

UPLC (Acid GEN_QC_SS): rt=0.52 minutes, peak observed: 325 (M+1) C₁₈H₂₀N₄O₂ requires 324.

### Description 47: methyl 2-chloro-6-methyl-3-pyridinecarboxylate (D47)

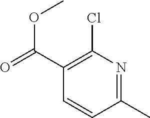

To a solution of 2-chloro-6-methyl-3-pyridinecarboxylic acid (8 g, 46.6 mmol) (available from Sigma-Aldrich #357847) in DCM (100 ml) and MeOH (50.0 ml) stirred under nitrogen at room temperature was added TMS-diazomethane 2 M in hexane (46.6 ml, 93 mmol). The reaction mixture was stirred at room temperature for 20 minutes. The solvents were removed to give the title compound D47 (7 g).

MS: (ES/+) m/z: 186 (M+1) C₈H₈ClNO₂ requires 185.

¹H NMR (400 MHz, CDCl₃) ppm 8.10 (d, 1H), 7.18 (d, 1H), 3.96 (s, 3H), 2.61 (s, 3H).

### Description 48: methyl 2-ethenyl-6-methyl-3-pyridinecarboxylate (D48)

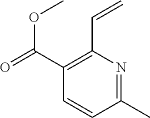

To a solution of methyl 2-chloro-6-methyl-3-pyridinecarboxylate D47 (2 g), Pd(Ph₃P)₄ (0.436 g, 0.377 mmol) in 1,4-Dioxane (15 ml) stirred under nitrogen at room temperature, was added tributyl(ethenyl)stannane (3.76 g, 11.85 mmol) neat in one charge. The reaction mixture was stirred at microwave Personal Chemistry at 100° C. for 30 minutes. The solvent was removed to give the crude product which was purified by flash cromatography on silica (Companion: 120 g column, gradient elution from Cy to Cy/EtOAc 1:1) to afford the title compound D48 (1.9 g).

UPLC (Basic GEN_QC: rt=0.73 minutes, peak observed: 178 (M+1) C₁₀H₁₁NO₂ requires 177.

¹H NMR (400 MHz, CDCl₃) δ ppm 8.08 (d, 1H), 7.66 (m, 1H), 7.12 (d, 1H), 6.52 (m, 1 H), 5.59 (m, 1H), 3.93 (s, 3H), 2.63 (s, 3H).

### Description 49: 2-ethenyl-6-methyl-3-(3-methyl-1,2,4-oxadiazol-5-yl)pyridine (D49)

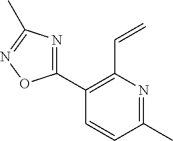

To a suspension of NaH 60% oil dispersion (0.903 g, 22.57 mmol) and molecular sieves in dry THF (10 ml) stirred under nitrogen at room temperature, acetamide oxime (0.836 g, 11.29 mmol) was added and the reaction stirred at room temperature for 30 minutes.

A solution of methyl 2-ethenyl-6-methyl-3-pyridinecarboxylate D48 (1 g) in dry THF (10 ml) was added in one charge. The reaction mixture was heated at the microwave Personal Chemistry at 100° C. for 30 minutes. NaHCO₃ saturated solution was added and the aqueous extracted with EtOAc; the organic phase was passed through a hydrophobic frit, the solvent removed to give the crude product which was purified by flash chromatography on silica (Companion: 80 g column, gradient elution from Cy to Cy/EtOAc 40:60) to afford the title compound D49 (308 mg).

UPLC (Basic GEN_QC: rt=0.78 minutes, peak observed: 202 (M+1) C₁₁H₁₁N₃O requires 201.

¹H NMR (400 MHz, CDCl₃) δ ppm 8.21 (d, 1H), 7.83 (m, 1H), 7.22 (d, 1H), 6.65 (m, 1 H), 5.69 (m, 1H), 2.67 (s, 3H), 2.52 (s, 3H).

### Description 50: 6-methyl-3-(3-methyl-1,2,4-oxadiazol-5-yl)-2-pyridinecarbaldehyde (D50)

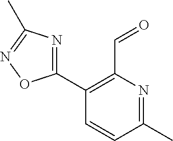

To a solution of 2-ethenyl-6-methyl-3-(3-methyl-1,2,4-oxadiazol-5-yl)pyridine D49 (100 mg) in THF (3 ml) and water (4.5 ml) stirred under nitrogen at room temperature, a solution of osmium tetroxide 4% in water (0.39 ml, 0.05 mmol) was added; after 5 minutes sodium periodate (319 mg, 1.491 mmol) was added in one charge. The reaction mixture was stirred at room temperature for 2 hours. The mixture was poured into a separatory funnel, washed with brine and the aqueous extracted with EtOAc. The phases were separated on a hydrophobic frit, the combined organic solvent was removed to give the crude product, which was purified by flash chromatography on silica gel (25 g column, gradient elution from Cy to Cy/EtOAc 80:20) to afford the title compound D50 (93 mg).

UPLC (Basic GEN_QC: rt 1=0.50 minutes and rt 2=0.55 minutes (rotamers present), peak observed: 204 (M+1) C₁₀H₉N₃O₂ requires 203.

¹H NMR (400 MHz, CDCl₃) δ ppm 10.55 (s, 1H), 8.21 (m, 1H), 7.53 (m, 1H), 2.78 (s, 3 H), 2.52-2.56 (m, 3H).

### Description 51: 6-methyl-3-(3-methyl-1,2,4-oxadiazol-5-yl)-2-pyridinecarboxylic acid (D51A/D51B)

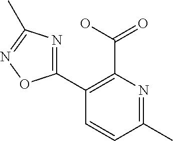

A) To a solution of 6-methyl-3-(3-methyl-1,2,4-oxadiazol-5-yl)-2-pyridinecarbaldehyde D50 (90 mg) in THF (3.00 ml) and water (6 ml) stirred at 0° C. was added solid NaOH (17.72 mg, 0.443 mmol) and after 10 minutes KMnO₄ (140 mg, 0.886 mmol) in one charge. The reaction mixture was stirred for 10 minutes. While still cold the reaction mixture was filtered on celite and the celite washed with HCl 1 M water solution and water. The aqueous filtrate at pH=1 was passed through a 50 g C18 column (MeOH, water to condition, water and then MeOH to elute) to afford the title compound DMA (70 mg).

UPLC (Basic GEN_QC: rt 1=0.50 minutes and rt 2=0.55 minutes (rotamers present), peak observed: 219 (M+1) C₁₀H₉N₃O₃ requires 218.

¹H NMR (400 MHz, CDCl₃) δ ppm 8.02 (d, 1H), 7.60 (d, 1H), 2.77 (s, 3H), 2.55 (s, 3H).

B) 6-methyl-3-(3-methyl-1,2,4-oxadiazol-5-yl)-2-pyridinecarbaldehyde D50 (0.89 g) was dissolved in a mixture of DMSO (10 ml) and pH=3 buffer solution (3 ml) and the solution was cooled to 0° C. A 1 M solution of NaClO₂ in water (16 ml) was added; the solution turned to pale yellow and after the addition was left stirring at room temperature for 2 hours. New pH=3 buffer solution (1.5 ml) was added and the stirring was continued for 1 hour. The mixture was eluted through a 70 g C18 cartridge (preconditioned with MeOH and then with water; eluted with water and then with MeOH). The methanol fractions were joined and evaporated under reduced pressure to afford the title compound D51B (0.89 g).

### Description 52: 2-chloro-N-(2-hydroxybutyl)-6-methyl-3-pyridinecarboxamide (D52)

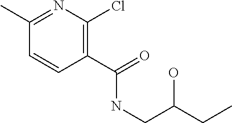

2-chloro-6-methyl-3-pyridinecarboxylic acid (2.5 g, 14.57 mmol) (available from Sigma-Aldrich #357847) was dissolved in DMF (35 ml) and DIPEA (7.63 ml, 43.7 mmol) was added. To this mixture TBTU (5.15 g, 16.03 mmol) was added in one portion and the resulting orange solution was stirred 45 minutes at room temperature. 1-amino-2-butanol (2.5 g, 28.0 mmol) was then added dissolved in DMF (5 ml) and the resulting mixture stirred at room temperature for 90 minutes. The mixture was then stored into the fridge overweekend. The mixture was partitioned between NaHCO₃ saturated solution and Et₂O; the water layer was extracted with Et₂O. The water layer was then extracted with EtOAc. The organic phases deriving from the Et₂O extractions were joined and dried over Na₂SO₄ and evaporated at reduced pressure; the oily residue was dried under high vacuum at 45° C. for 2 hours, obtaining a first batch of crude material purified by flash chromatography on silica gel (Biotage 100 g column, EtOAc/Cy from 30:70 to 75:25). The organic phases deriving from the EtOAc extractions were joined, dried over Na₂SO₄ and evaporated at reduced pressure; the oily residue was dried under high vacuum at 45° C. for 1 hour, obtaining a second batch of crude material, purified by flash chromatography on silica gel (Biotage 340 g column, EtOAc/Cy from 30:70 to 75:25). The fractions eluted performing the two purifications were joined together and then evaporated at reduced pressure, to obtain the title compound D52 as pale yellow oil (3.62 g).

MS: (ES/+) m/z: 243 (M+1) C₁₁H₁₅ClN₂O₂ requires 242.

¹H NMR (400 MHz, DMSO-d₆) δ ppm 8.45 (m, 1H), 7.77 (m, 1H), 7.33 (m, 1H), 4.69 (m, 1H), 3.43-3.61 (m, 1H), 3.05-3.30 (m, 2H), 2.48 (s, 3H), 1.51 (m, 1H), 1.18-1.42 (m, 1H), 0.90 (t, 3H)

### Description 53: 2-chloro-6-methyl-N-(2-oxobutyl)-3-pyridinecarboxamide (D53)

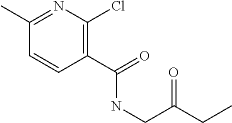

2-chloro-N-(2-hydroxybutyl)-6-methyl-3-pyridinecarboxamide D52 (3.62 g) was dissolved in DCM (100 ml) then, to the stirred solution, Dess-Martin periodinane (6.75 g, 15.91 mmol) was added portionwise over 5 minutes. The mixture was stirred at room temperature for 45 minutes (white suspension). The mixture was then partitioned between NaHCO₃ saturated solution and DCM; water layer was extracted with DCM. The organic phases were joined, dried over Na₂SO₄ and evaporated at reduced pressure, obtaining the crude target material as pale yellow solid (7.2 g). This material was stored in the fridge overnight and was purified by flash chromatography on silica gel (Snap-340 g column, EtOAc/Cy from 20:80 to 80:20) to give the title compound D53 (3.11 g) as white solid.

MS: (ES/+) m/z: 241 (M+1) C₁₁H₁₃ClN₂O₂ requires 240.

¹H NMR (400 MHz, DMSO-d₆) δ ppm 8.82 (m, 1H), 7.81 (m, 1H), 7.37 (m, 1H), 4.09 (d, 2H), 3.30-3.35 (s, 3H), 2.53-2.59 (m, 2H), 0.97 (t, 3H).

### Description 54: 2-chloro-3-(5-ethyl-1,3-oxazol-2-yl)-6-methylpyridine (D54)

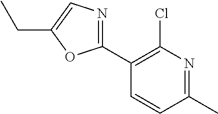

2-Chloro-6-methyl-N-(2-oxobutyl)-3-pyridinecarboxamide D53 (3.051 g) was dissolved in THF (100 ml) and Burgess reagent (3.104 g, 13.03 mmol) was added in one portion. The pale yellow solution was stirred at room temperature for 4.5 hours, then new Burgess reagent (0.41 g, 1.72 mmol) was added and the mixture stirred at 60° C. for 1.5 hours.

The solvent was evaporated at reduced pressure and the residue partitioned between NaHCO₃ saturated solution and EtOAc; water layer was extracted with EtOAc. The organic phases were joined, dried over Na₂SO₄ and evaporated at reduced pressure, obtaining the crude target material, which was then purified by flash chromatography on silica gel (Snap-100 g column, EtOAc/Cy from 20:80 to 90:10). After evaporation at reduced pressure it was obtained the title compound D54 (1.7 g) as colorless oil, which slowly solidified upon standing at room temperature.

MS: (ES/+) m/z: 223 (M+1) C₁₁H₁₁ClN₂O requires 222.

¹H NMR (400 MHz, CDCl₃) δ ppm 8.21 (d, 1H), 7.21 (d, 1H), 6.96 (s, 1H), 2.80 (m, 2H), 2.62 (s, 3H), 1.35 (t, 3H).

### Description 55: 2-ethenyl-3-(5-ethyl-1,3-oxazol-2-yl)-6-methylpyridine (D55)

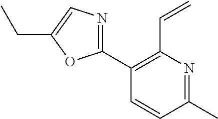

2-chloro-3-(5-ethyl-1,3-oxazol-2-yl)-6-methylpyridine D54 (168 mg), Pd(Ph₃P)₄ (70 mg, 0.061 mmol), 2-ethenyl-4,4,5,5-tetramethyl-1,3,2-dioxaborolane (0.2 ml, 1.179 mmol) and K₂CO₃ (209 mg, 1.509 mmol) were mixed together, then 1,4-dioxane (8 ml) and water (3 ml) were added. The mixture was stirred at 80° C. for 30 minutes. The mixture was stirred again at 80° C. for other 50 minutes. The solvents were evaporated at reduced pressure and the residue partitioned between NaHCO₃ saturated solution and Et₂O; water layer extracted with Et₂O. The organic phases were joined, dried over Na₂SO₄ and evaporated at reduced pressure, obtaining the crude target material which was purified by flash chromatography on silica gel (Snap-25 g column, EtOAc/Cy from 5:95 to 30:70). It was obtained the title compound D55 (135 mg) as white solid.

MS: (ES/+) m/z: 215 (M+1) C₁₃H₁₄N₂O requires 214.

¹H NMR (400 MHz, CDCl₃) δ ppm 8.10 (m, 1H), 7.87 (m, 1H), 7.15 (m 1H), 6.92 (s, 1 H), 6.56 (m, 1H), 5.61 (m, 1H), 2.68-2.87 (m, 2H), 2.63 (s, 3H), 1.34 (t, 3H).

### Description 56: 3-(5-ethyl-1,3-oxazol-2-yl)-6-methyl-2-pyridinecarbaldehyde (D56)

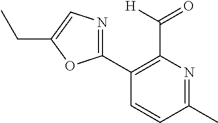

2-Ethenyl-3-(5-ethyl-1,3-oxazol-2-yl)-6-methylpyridine D55 (132 mg) was dissolved in THF (3 ml) and water (3 ml). To this stirred mixture a solution of osmium tetroxide 4% in water (0.390 ml, 0.050 mmol) was added over 30 seconds and the resulting mixture was then stirred at room temperature for 5 minutes. Sodium periodate (329 mg, 1.538 mmol) was then added in one portion and the resulting mixture was left stirring at room temperature for 70 minutes. The mixture was then partitioned between NaHCO₃ saturated solution and Et₂O; aqueous layer was extracted with Et₂O. The organic phases were joined, dried over Na₂SO₄ and evaporated at reduced pressure, obtaining the title compound D56 (136 mg) as brown solid.

MS: (ES/+) m/z: 217 (M+1) C₁₂H₁₂N₂O₂ requires 216.

¹H NMR (400 MHz, CDCl₃) δ ppm 10.75 (s, 1H), 8.25 (d, 1H), 7.45 (d, 1H), 6.98 (s, 1H), 2.76-2.91 (m, 2H), 2.74 (s, 3H), 1.35 (t, 3H).

### Description 57: 3-(5-ethyl-1,3-oxazol-2-yl)-6-methyl-2-pyridinecarboxylic acid (D57)

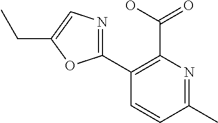

3-(5-ethyl-1,3-oxazol-2-yl)-6-methyl-2-pyridinecarbaldehyde D56 (550 mg) was dissolved in DMSO (5 ml) and citric pH=3 buffer solution (1.5 ml) and the mixture was chilled at 0° C. NaClO₂ 1 M in water (7 ml, 7.00 mmol) was dropped into the mixture over 10 minutes, then the stirring was continued at room temperature. New citric pH=3 buffer solution (1.5 ml), followed by new NaClO₂ 1 M in water (3 ml, 3.00 mmol) were dropped into the mixture, which was then stirred at room temperature for other 30 minutes, then the whole mixture was stored in the fridge overnight. NaClO₂ 1 M in water (1 ml, 3.00 mmol) was dropped into the mixture, which was then stirred at room temperature for other 30 minutes. The whole dark mixture was loaded onto a C18-70 g column (firstly eluted with water then with MeOH). After evaporation at reduced pressure of the methanol fractions it was obtained the crude dark brown oil, which solidified by Et₂O (2 ml) addition. To this solid acetone (2.5 ml) and Et₂O (3 ml) were added. The solid was filtered and dried under high vacuum for 30 minutes, giving a dark brown solid (23 mg). To the solution Et₂O (8 ml) was added and the so obtained mixture was stored for 70 minutes into the fridge. The solid was filtered and washed with Et₂O (3 ml). All the organic solution (mother liquor and Et₂O of washing) were joined, evaporated at reduced pressure and dried under high vacuum at 45° C. for 30 minutes, giving the title compound D57 (362 mg) as brown gum.

MS: (ES/−) m/z: 231 (M−1) C₁₂H₁₂N₂O₃ requires 232.

¹H NMR (400 MHz, DMSO-d₆) δ ppm 8.20 (d, 1H), 7.50 (d, 1H), 7.05 (s, 1H), 2.61-2.82 (m, 3H), 2.55 (s, 3H), 1.23 (m, 3H).

### Description 58: 6-methyl-3-(2-pyrimidinyl)-2-pyridinecarbonitrile (D58)

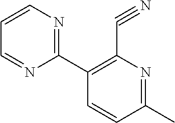

Isopropylmagnesium chloride LiCl (37.9 ml, 36.5 mmol) was added portion wise (in overall 10 minutes) to a solution of 3-bromo-6-methyl-2-pyridinecarbonitrile (4 g, 20.30 mmol) in THF (150 ml) cooled to −70° C. (internal temperature). The reaction was kept to that temperature for 15 minutes, then it was allowed to gently warm up to −40° C. in overall 1 hour. Then, it was cooled to −78° C. and zinc chloride (3.32 g, 24.36 mmol) was added. The resulting mixture was allowed to warm up to room temperature in 1 hour. Pd(Ph₃P)₄ (2.346 g, 2.030 mmol), 2-chloropyrimidine (3 g, 26.2 mmol) were added and the mixture was refluxed (external temperature 100° C.) until complete consumption of starting choropyrimidine (3 hours). The reaction mixture was cooled to room temperature and poured into water (200 ml) cooled to 10° C. It was then extracted with EtOAc. The collected organic phases, containing large amount of colloid material and water, were washed with brine (200 ml). The aqueous phase was filtered over a gouch, and the solid material was washed with further EtOAc. The collected organic phases were dried overnight over Na₂SO₄, filtered and concentrated to give the crude material (7 g) which was purified (Biotage Sp1 over a 240 g Silica Analogix column, with a 25 g pre-column) to give the title compound D58 as yellow solid (1.8 g).

UPLC (Acid GEN_QC_SS): rt=0.58 minutes, peak observed: 197 (M+1) C₁₁H₈N₄ requires 196.

### Description 59: 6-methyl-3-(2-pyrimidinyl)-2-pyridinecarboxylic acid (D59)

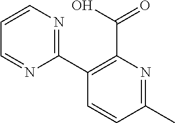

6-methyl-3-(2-pyrimidinyl)-2-pyridinecarbonitrile D58 (0.8 g) was reacted in 6 M aqueous HCl (40 ml, 240 mmol) at 80° C. for 3 hours, then solvent was removed under vacuum, and the resulting crude was purified (70 g Varian C18 column, conditioning with MeOH, then water, loading in water, washing with water). The title compound D59 was recovered as yellow solid (0.6 g).

UPLC (Acid GEN_QC_SS): rt=0.30 minutes, peak observed: 216 (M+1) C₁₁H₉N₃O₂ requires 217.

¹H NMR (400 MHz, DMSO-d₆) δ ppm 13.07 (bs, 1H), 8.78-9.01 (m, 2H), 8.39 (m, 1H), 7.39-7.67 (m, 2H), 2.56-2.67 (s, 3H).

### Description 60: (1R,4S,6R)-4-{[(1,1-dimethylethyl)(diphenyl)silyl]oxy}methyl)-3-{[6-methyl-3-(3-methyl-1,2,4-oxadiazol-5-yl)-2-pyridinyl]carbonyl}-3-azabicyclo[4.1.0]heptane (D60)

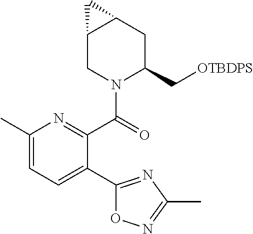

To a solution of 6-methyl-3-(3-methyl-1,2,4-oxadiazol-5-yl)-2-pyridinecarboxylic acid D51 (400 mg) in dry DMF (5 ml) under N₂ atmosphere, TBTU (585 mg, 1.822 mmol) and DIPEA (0.398 ml, 2.277 mmol) were added; mixture became black and was stirred at room temperature for 20 minutes.

A solution of (1R,4S,6R)-4-({[(1,1-dimethylethyl)(diphenyl)silyl]oxy}methyl)-3-azabicyclo[4.1.0]heptane D10 (555 mg) in DMF (3 ml) was added and the reaction was left stirring at room temperature for 3 hours.

Mixture was diluted with EtOAc and washed with NaHCO₃ saturated solution and water. Organics were dried and evaporated, and the crude was purified by Si flash chromatography (SNAP 25 g) eluting with Cy/EtOAc 1:1, affording the title compound D60 (640 mg) as white foam.

UPLC (Acid GEN_QC_SS): rt1=1.25 minutes and rt2=1.27 minutes (rotamers present) peak observed: 567 (M+1) C₃₃H₃₈N₄O₃Si requires 566.

### Description 61: ((1R,4S,6R)-3-{[6-methyl-3-(3-methyl-1,2,4-oxadiazol-5-yl)-2-pyridinyl]carbonyl}-3-azabicyclo[4.1.0]hept-4-yl)methanol (D61)

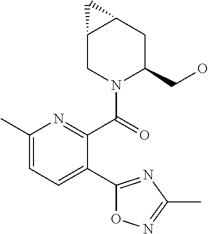

To a solution of (1R,4S,6R)-4-({[(1,1-dimethylethyl)(diphenyl)silyl]oxy}methyl)-3-{[6-methyl-3-(3-methyl-1,2,4-oxadiazol-5-yl)-2-pyridinyl]carbonyl}-3-azabicyclo[4.1.0]heptane D60 (0.64 g) in dry THF (7 ml) at room temperature under N₂ flux, TBAF (1.129 ml, 1.129 mmol) was added and the mixture was stirred at room temperature for 2 hours.

Volatiles were removed under reduced pressure and the resulting crude was purified by Si flash chromatography (SNAP 10 g) eluting with EtOAc 100%, affording the title compound D61 (335 mg) as white solid.

UPLC (Acid GEN_QC_SS): rt1=0.60 minutes and rt2=0.62 minutes (rotamers present) peak observed: 329 (M+1) C₁₇H₂₀N₄O₃ requires 328.

### Description 62: (1R,4S,6R)-4-{[(1,1-dimethylethyl)(diphenyl)silyl]oxy}methyl)-3-{[6-methyl-3-(2H-1,2,3-triazol-2-yl)-2-pyridinyl]carbonyl}-3-azabicyclo[4.1.0]heptanes (D62)

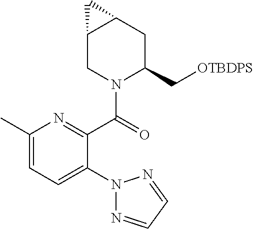

TBTU (97 mg, 0.301 mmol) was added to a stirred solution of (1R,4S,6R)-4-({[(1,1-dimethylethyl)(diphenyl)silyl]oxy}methyl)-3-azabicyclo[4.1.0]heptane D10 (100 mg), 6-methyl-3-(2H-1,2,3-triazol-2-yl)-2-pyridinecarboxylic acid D33 (55.9 mg) and DIPEA (0.057 ml, 0.328 mmol) in DCM (3 ml) at room temperature. The reaction was stirred for 1 hour. The reaction was quenched with saturated NaHCO₃ solution (30 ml), and extracted with EtOAc (2×30 ml). The combined organic phases were washed with water (30 ml), brine (20 ml), dried (Na₂SO₄) and evaporated under reduced pressure, to give a colourless residue which was purified via Biotage (SNAP 28 g KP—NH column 2-5% iPrOH/cyclohexane, and then SNAP 25 SiO₂ column, EtOAc isocratic) to give the title compound D62 (140 mg) as colourless oil.

UPLC (Acid GEN_QC: rt1=1.13 minutes and rt2=1.15 minutes (rotamers present) peak observed: 552 (M+1) C₃₂H₃₇N₅O₂Si requires 551.

¹H NMR (400 MHz, CHLOROFORM-d) δ ppm 0.43-0.64 (m, 2H) 0.73-0.99 (m, 2H) 1.11 (s, 9H) 1.75-1.88 (m, 1H) 2.30-2.40 (m, 1H) 2.61 (s, 3H) 3.23-4.02 (m, 4H) 4.63-4.74 (m, 1H) 7.18-7.83 (m, 13H) 8.16 (d, 1H)

### Description 63: ((1R,4S,6R)-3-{[6-methyl-3-(2H-1,2,3-triazol-2-yl)-2-pyridinyl]carbonyl}-3-azabicyclo[4.1.0]hept-4-yl)methanol (D63)

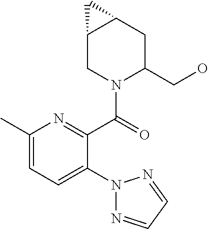

1 M TBAF in THF (0.266 ml, 0.266 mmol) was added to a stirred solution of (1R,4S,6R)-4-({[(1,1-dimethylethyl)(diphenyl)silyl]oxy}methyl)-3-{[6-methyl-3-(2H-1,2,3-triazol-2-yl)-2-pyridinyl]carbonyl}-3-azabicyclo[4.1.0]heptane D62 (140 mg) in THF (5 ml) at room temperature and the reaction mixture was stirred overnight. The reaction mixture was evaporated under reduced pressure and the residue was partitioned between EtOAc (40 ml) and saturated NH₄Cl solution (20 ml). The phases were separated and the aqueous one was extracted with EtOAc (20 ml). The combined organic phases were washed with water (40 ml) and brine (30 ml), dried (Na₂SO₄) and evaporated under reduced pressure to give a colourless gummy residue which was purified via Biotage (SNAP 25 g SiO₂ column, MeOH/DCM 2-10%) to give the title compound D63 (78 mg) as colourless gum.

UPLC (Acid GEN_QC: rt1=0.54 minutes and rt2=0.55 minutes (rotamers present) peak observed: 314 (M+1) C₁₆H₁₉N₅O₂ requires 313.

¹H NMR (400 MHz, CHLOROFORM-d) δ ppm 0.52-0.59 (m, 1H) 0.78-0.86 (m, 1H) 0.99-1.21 (m, 2H) 1.94-2.12 (m, 2H) 2.64 (s, 3H) 3.39-4.12 (m, 4H) 4.31-4.63 (m, 1 H) 4.75 (d, 1H) 7.35 (d, 1H) 7.86 (s, 2H) 8.22 (d, 1H)

### Description 64: 5-bromo-2-pyrazinamine (D64)

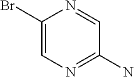

In a 2 L, ice-bath cooled (NaCl) round-bottomed flask, 2-pyrazinamine (30 g, 315 mmol) was dissolved in DCM (850 ml) NBS (59.0 g, 331 mmol) was added and the reaction mixture was left stirring at 0° C. for 1 hour. The mixture was let to warm-up to room temperature and was left stirring for 1 hour. Solvent was evaporated and the crude was purified by Silica Pad eluting with DCM 100% to DCM/MeOH 90:10. The recovered product was triturated with cyclohexane. The resulting pale yellow solid was filtered through a gouch funnel and dried under vacuum to give the title compound D64 (23.6 g). Mother liquors were concentrated under vacuum to give a second batch of title compound D64 (4.8 g). Impure fractions coming from the silica pad were purified by Silica Chromatography (Biotage SP—column size 340 g) using DCM 100% to DCM/MeOH 90:10 as eluent. It was recovered a third batch of title compound D64 (7.2 g).

UPLC (Acid Final_QC): rt=0.54 minutes, peak observed: 174 (M) C₄H₄BrN₃ requires 174.

¹H NMR (400 MHz, DMSO-d₆) δ ppm 6.64 (br. s., 2H) 7.70 (d, 1H) 8.04 (d, 1H)

### Description 65: 2-bromo-5-iodopyrazine (D65)

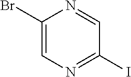

In a 1000 ml, ice bath cooled round-bottomed flask was dissolved 5-bromo-2-pyrazinamine D64 (11.3 g) in Acetonitrile (125 ml)/water (188 ml). In the mixture HI 67% water solution (45 ml, 401 mmol) was added. To the solution was added dropwise during 150 minutes a solution of sodium nitrite (31.4 g, 455 mmol) in water (125 ml). After the addition the reaction mixture was sealed, was let to warm-up to room temperature and was heated at 50° C. for 30 hours. After cooling the mixture was poured into 800 ml of 20% NaOH and was extracted with Et₂O (3×800 ml). Gathered Et₂O layers were washed with Na₂S₂O₅ saturated solution, dried over Na₂SO₄ and the solvent was evaporated. The crude was purified by Silica Chromatography (Biotage SP—column size 340 g) using DCM 100% to DCM/MeOH 90:10 as eluent. It was recovered the title compound D65 (896 mg) as yellow powder.

¹H NMR (400 MHz, CHLOROFORM-d) δ ppm 8.49-8.56 (m, 1H) 8.65 (d, 1H)

### Description 66: 2-bromo-5-(trifluoromethyl)pyrazine (D66)

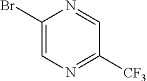

Potassium fluoride (238 mg, 4.09 mmol) and copper(I) iodide (779 mg, 4.09 mmol) were mixed and heated under vacuum using heat gun (temperature 360° C., on display of heating gun) for 20 minutes (until a greenish colour of the mixture appeared). After cooling at room temperature, DMF (4 ml) and NMP (4.00 ml) were added followed by (trifluoromethyl)trimethylsilane (0.603 ml, 3.77 mmol) and 2-bromo-5-iodopyrazine D65 (896 mg). The resulting mixture was stirred at room temperature for 5 hours. The reaction mixture was poured in 200 ml of 6N NH₃ water solution and was extracted twice with Et₂O (3×50 ml). Gathered Et₂O layers were dried over Na₂SO₄.

Diethyl ether was distilled by Claisen apparatus. It was recovered the title compound D66 (586 mg).

¹H NMR (400 MHz, CHLOROFORM-d) δ ppm 8.73-8.81 (m, 1H) 8.84 (s, 1H)

### Description 67: 1,1-dimethylethyl (1R,4S,6R)-4-({[5-(trifluoromethyl)-2-pyrazinyl]oxy}methyl)-3-azabicyclo[4.1.0]heptane-3-carboxylate (D67)

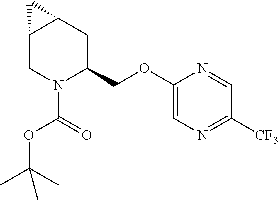

To a solution of 1,1-dimethylethyl (1R,4S,6R)-4-(hydroxymethyl)-3-azabicyclo[4.1.0]heptane-3-carboxylate D20 (120 mg) and 2-bromo-5-(trifluoromethyl)pyrazine D66 (120 mg) in DMF (4 ml) at 0° C. (ice bath) was added NaH (31.7 mg, 0.792 mmol) (gas evolution). The reaction mixture was slowly warmed to room temperature and stirred at room temperature for 1 hour. The reaction was quenched by a slow and careful addition of a saturated aqueous solution of NaHCO₃ (40 ml). The organic phase was extracted with DCM (3×50 ml); the joined organic phase was washed with water and brine, dried over Na₂SO₄, filtered and concentrated to give the crude material. This was purified by Silica Chromatography (Biotage SP—column size 25 g) using Cy/EtOAc 90:10 as eluent. The appropriate fractions were concentrated to obtain the title compound D67 (62 mg).

UPLC (Basic GEN_QC): rt=1.07 minutes, peak observed: 374 (M+1) C₁₇H₂₂F₃N₃O₃ requires 374.

### Description 68: (1R,4S,6R)-4-({[5-(trifluoromethyl)-2-pyrazinyl]oxy}methyl)-3-azabicyclo[4.1.0]heptane (D68)

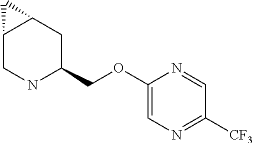

To a solution of 1,1-dimethylethyl (1R,4S,6R)-4-({[5-(trifluoromethyl)-2-pyrazinyl]oxy}methyl)-3-azabicyclo[4.1.0]heptane-3-carboxylate D67 (62 mg) in DCM (1.5 ml) at room temperature TFA (0.75 ml, 9.73 mmol) was added dropwise. The solution was stirred at room temperature for 1 hour. The volatiles were removed under reduced pressure.

The residue was dissolved in DCM and then was loaded onto a SCX column and eluted with methanol and ammonia 2M in methanol. It was recovered the title compound D68 (35 mg).

UPLC (Acid Final_QC): rt=0.47 minutes, peak observed: 274 (M+1) C₁₂H₁₄F₃N₃O requires 273.

### Description 69: 1,1-dimethylethyl (1R,4S,6R)-4-formyl-3-azabicyclo[4.1.0]heptane-3-carboxylate (D69)

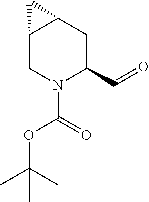

To a solution of 1,1-dimethylethyl (1R,4S,6R)-4-(hydroxymethyl)-3-azabicyclo[4.1.0]heptane-3-carboxylate D20 (11 g) in DCM (200 ml) at room temperature, Dess-Martin periodinane (22 g, 51.9 mmol) was added portion-wise and the reaction was stirred at room temperature overnight. TLC check (Cy/EtOAc 1:1) showed the disappearance of starting material. The reaction was quenched with Na₂S₂O₃ 5% solution in NaHCO₃ saturated solution (500 ml). The mixture was stirred vigorously for 2 hours, extracted with DCM (3×400 ml), filtered on a phase separator and concentrated to obtain 12 g of yellow oil.

It was purified by Silica flash chromatography (SP1, SNAP 340 g Si cartridge, eluting mixture Cy/EtOAc (from 95:5 to 70:30, 10 CV)), to obtain the title compound D69 (9.18 g) as yellow oil.

UPLC (Acid GEN_QC_SS): rt=0.83 minutes, peaks observed: 226 (M+1) 170 [M+1-C(CH₃)₃], C₁₂H₁₉NO₃ required 225

¹H NMR (500 MHz, CHLOROFORM-d) δ ppm 0.22-0.27 (m, 1H) 0.64-0.74 (m, 1H) 0.91-1.03 (m, 1H) 1.07-1.16 (m, 1H) 1.43 (s, 9H) 1.65-1.72 (m, 1H) 2.26-2.34 (m, 1 H) 3.51 (dd, 1H) 3.84 (dd, 1H) 3.95-4.03 (m, 1H) 9.49-9.57 (m, 1H)

### Description 70:1,1-dimethylethyl (1R,4S,6R)-4-ethenyl-3-azabicyclo[4.1.0]heptane-3-carboxylate (D70)

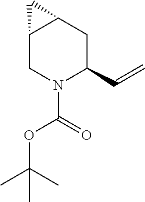

To a suspension of methyl(triphenyl)phosphonium bromide (3.78 g, 10.59 mmol) in THF (40 ml) (white, quite homogeneous) at room temperature, 1.6M BuLi in Hexane (6.62 ml, 10.59 mmol) was added dropwise. The suspension became orange.

The reaction mixture was stirred for 10 minutes, then a solution of 1,1-dimethylethyl (1R,4S,6R)-4-formyl-3-azabicyclo[4.1.0]heptane-3-carboxylate D69 (1 g) in THF (10 ml) was added. The resulting mixture became dark orange and was stirred at room temperature overnight.

The reaction was quenched with NaHCO₃ saturated solution (100 ml) and extracted with EtOAc (3×60 ml). The organic phase was filtered by a phase separator and concentrated, to obtain 2.75 g of a dark yellow oil.

It was purified by Si flash chromatography (SP1, Snap 100 g Si cartridge, eluting mixture Cy/EtOAc from 0% to 5% EtOAc, 10 CV) to obtain the title compound D70 (0.848 g) as colourless oil.

UPLC (IPQC): rt=1.25 minutes, peak observed: 224 (M+1), C₁₃H₂₁NO₂ requires 223

¹H NMR (400 MHz, CHLOROFORM-d) δ ppm 0.02-0.18 (m, 1H) 0.60-0.72 (m, 1H) 0.88-1.02 (m, 2H) 1.45 (s, 9H) 1.67-1.77 (m, 1H) 2.00-2.12 (m, 1H) 3.46-3.58 (m, 1 H) 3.67-3.76 (m, 1H) 4.26-4.55 (m, 1H) 5.09-5.22 (m, 2H) 5.79-5.91 (m, 1H)

### Description 71: 1,1-dimethylethyl (1R,4S,6R)-4-(2-hydroxyethyl)-3-azabicyclo[4.1.0]heptane-3-carboxylate (D71)

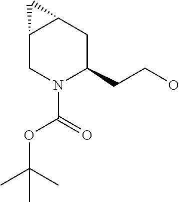

To a solution of 1,1-dimethylethyl (1R,4S,6R)-4-ethenyl-3-azabicyclo[4.1.0]heptane-3-carboxylate D70 (3.5 g) in dry THF (70 ml) under Argon at 0° C., 9-BBN 0.5M in THF (47.0 ml, 23.51 mmol) was added dropwise (slightly exothermic). After 2 hours further 9-BBN 0.5M in THF (47.0 ml, 23.51 mmol) was added and after 1 hour the reaction mixture was allowed to reach room temperature. After overall 6 hours TLC showed almost complete conversion. Mixture (homogeneous and colourless) was cooled down to −15° C. and dropwise treated subsequently with H₂O₂ 30% (31 ml, 303 mmol) (caution exothermic, internal temperature maintained below 10° C.) and NaOH 1M (31 ml, 31.0 mmol). The resulting slurry was left stirring overnight at room temperature. Mixture was taken up with a mixture of EtOAc (250 ml)/Et₂O (250 ml)/water (250 ml). Phases were separated and the aqueous one was back extracted with EtOAc/Et₂O 1:1 (2×200 ml). Combined organic phases were dried over Na₂SO₄ and evaporated to dryness to get 10 g of crude material as colourless oil.

It was purified over Si flash chromatography (Biotage SNAP 340 g column eluting with Cy/EtOAc 85:15 to 40:60 in 15 CV). Evaporation of solvent afforded the title compound D71 (3.2 g).

UPLC (IPQC): rt=0.93 minutes, peak observed: 242 (M+1) C₁₃H₂₃NO₃ requires 241.

### Description 72: 1,1-dimethylethyl (1R,4S,6R)-4-{2-[(5-fluoro-2-pyridinyl)oxy]ethyl}-3-azabicyclo[4.1.0]heptane-3-carboxylate (D72)

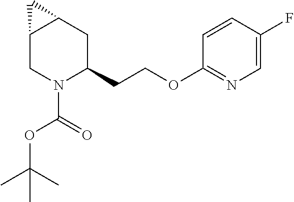

To a solution of 1,1-dimethylethyl (1R,4S,6R)-4-(2-hydroxyethyl)-3-azabicyclo[4.1.0]heptane-3-carboxylate D71 (0.640 g), 5-fluoro-2(1H)-pyridinone (0.450 g, 3.98 mmol) and tributylphosphine (1.309 ml, 5.30 mmol) in THF (40 ml) at 40° C., DEAD (1.221 g, 5.30 mmol) was added and the reaction mixture was stirred at 39° C. for 30 minutes. The reaction mixture was concentrated to obtain a crude yellow oil (3.65 g).

The crude was purified by Si flash chromatography (SP1, SNAP 100 g Si cartridge, eluting mixture Cy/EtOAc from 10:0 to 9:1, 3 CV—9:1, 7 CV), to obtain the title compound D72 (0.673 g) as colourless oil.

UPLC (IPQC): rt=1.35 minutes, peak observed: 337 (M+1), C₁₈H₂₅FN₂O₃ requires 336.

### Description 73: (1R,4S,6R)-4-{2-[(5-fluoro-2-pyridinyl)oxy]ethyl}-3-azabicyclo[4.1.0]heptane (D73)

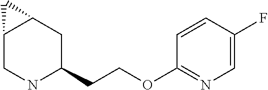

To a solution of 1,1-dimethylethyl (1R,4S,6R)-4-{2-[(5-fluoro-2-pyridinyl)oxy]ethyl}-3-azabicyclo[4.1.0]heptane-3-carboxylate D72 (0.673 g) in DCM (20 ml) at 0° C., TFA (5 ml, 64.9 mmol) was added and the reaction was stirred, allowing the mixture to reach room temperature, for 1 hour. Solvent was concentrated. The crude obtained was dissolved in MeOH and loaded on a 5 g SCX cartridge and eluted with MeOH/NH₃ 2M in MeOH. Ammoniacal fractions were gathered and concentrated to obtain the title compound D73 as whitish solid (0.457 g).

UPLC (IPQC): rt=0.53 minutes, peak observed 237 (M+1), C₁₃H₁₇FN₂O requires 236.

### Description 74: N-(4-chloro-2-pyridinyl)-2,2-dimethylpropanamide (D74)

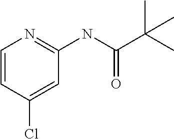

2,2-dimethylpropanoyl chloride (7.03 g, 58.3 mmol) was added to a solution of 4-chloro-2-pyridinamine (5 g, 38.9 mmol) in pyridine (20 ml) keeping the internal temperature below 35° C. The resulting mixture was stirred at room temperature overnight, then it was taken up with EtOAc (300 ml) and washed with water (2×100 ml). The organic phase was dried over Na₂SO₄, filtered and concentrated to give a yellow solid. This was dissolved in EtOH (30 ml), and stored at 4° C. overnight. The resulting solid was filtered washing with cold EtOH to give a first batch of title compound D74 as colourless solid (1.4 g). The filtrate was concentrated and taken up with EtOH (20 ml) and left on standing at 4° C. to give a second batch of D74 as nice colourless crystals (650 mg). The solution was concentrated to give a third batch of D74 (5.5 g).

UPLC (Acid GEN_QC_SS): rt=0.81 minutes, peak observed: 213 (M+1) C₁₀H₁₃ClN₂O requires 212.

¹H NMR (400 MHz, CHLOROFORM-d) δ ppm 1.30-1.41 (m, 9H) 7.07 (dd, 1H) 8.08 (br. s., 1H) 8.18 (d, 1H) 8.39 (d, 1H)

### Description 75: N-(4,5-dichloro-2-pyridinyl)-2,2-dimethylpropanamide (D75)

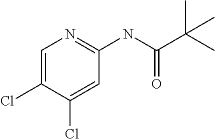

N-(4-chloro-2-pyridinyl)-2,2-dimethylpropanamide D74 (1.5 g) was reacted with NCS (4.71 g, 35.3 mmol) in Acetonitrile (50 ml) at reflux for 5 hours, then solvent was removed under vacuum, rinsed with DCM (200 ml) and washed with 10% aqueous NaOH (2×30 ml) and water (2×50 ml), dried over Na₂SO₄, filtered and concentrated. The resulting solid was crystallised from EtOH to give a first batch of title compound D75 (0.860 mg). The solution was further concentrated to 20 ml and left on standing at 4° C. for 3 days. Then it was filtered to give a second batch of title compound D75 (200 mg), and the solution concentrated to give a crude (450 mg) that was purified with Biotage Sp1 over a 50 g silica SNAP column, with a gradient of Cy/EtOAc. The title compound was eluted with ca 15% EtOAc, obtaining a third batch of title compound. This was collected with the second batch to give a fourth batch of title compound D75 (560 mg).

UPLC (Acid GEN_QC_SS): rt=0.95 minutes, peak observed: 247 (M+1) C₁₀H₁₂Cl₂N₂O requires 246.

¹H NMR (400 MHz, CHLOROFORM-d) δ ppm 1.29-1.42 (m, 9H) 8.03 (br. s., 1H) 8.29 (s, 1H) 8.52 (s, 1H)

### Description 76: 4,5-dichloro-2-pyridinamine (D76)

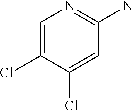

N-(4,5-dichloro-2-pyridinyl)-2,2-dimethylpropanamide D75 (560 mg) was reacted with HCl (10 ml, 60.0 mmol) at 80° C. for 1 hour then it was purified over a 20 g SCX Strata column, washing with MeOH and eluting with 2M ammonia in MeOH, to give the title compound D76 (360 mg) as colourless solid.

¹H NMR (400 MHz, CHLOROFORM-d) δ ppm 4.54 (br. s., 2H) 6.63 (s, 1H) 8.09 (s, 1H)

### Description 77: 2,4,5-trichloropyridine (D77)

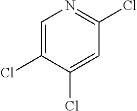

4,5-dichloro-2-pyridinamine D76 (360 mg) was dissolved in HCl (8 ml, 96 mmol) at 0° C., then sodium nitrite (305 mg, 4.42 mmol) was added portionwise, and the resulting yellow mixture was stirred at 0° C. for 1 hour and then at room temperature for 1 hour. On the basis of HPLC/MS, starting material was consumed to give the required product and the corresponding pyridone.

The reaction was then poured into ammonium hydroxide (10 ml) in ice and extracted with Et₂O (3×150 ml). The collected organic phases were dried over Na₂SO₄, then filtered and carefully concentrated (max 200 mBar) to give the title compound D77 (200 mg) as yellow oil.

UPLC (Acid GEN_QC_SS): rt=0.86 minutes, peak observed: 184 (M+2) C₅H₂Cl₃N requires 182.

¹H NMR (400 MHz, CHLOROFORM-d) δ ppm 7.48 (s, 1H) 8.43 (s, 1H)

### Description 78: (1R,4S,6R)-4-{[(1,1-dimethylethyl)(diphenyl)silyl]oxy}methyl)-3-[(6-methyl-2-pyridinyl)carbonyl]-3-azabicyclo[4.1.0]heptane (D78)

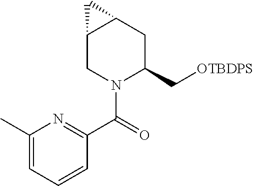

Into a 50 ml round bottomed flask 6-methyl-2-pyridinecarboxylic acid (0.188 g, 1.368 mmol) was added and dissolved in DCM (20 ml). To this solution DIPEA (1.433 ml, 8.21 mmol) and TBTU (0.483 g, 1.504 mmol) were added and the resulting mixture was stirred at room temperature for 30 minutes. Then a DCM solution (5 ml) of (1R,4S,6R)-4-({[(1,1-dimethylethyl)(diphenyl)silyl]oxy}methyl)-3-azabicyclo[4.1.0]heptane D10 (0.5 g) was added and the resulting mixture left under stirring at room temperature for 12 hours. After this time additional 1.1 equivalent of TBTU (0.483 g, 1.504 mmol) was added and the reaction mixture was left under stirring at room temperature for 3 hours. The solution was transferred into a reparatory funnel containing brine (50 ml) and it was extracted with DCM (4×25 ml). The collected organic phases were dried (Na₂SO₄) and solvent evaporated to give a slightly orange oil. This material was purified by column chromatography on silica gel (flash master, 50 g Si cartridge, eluting with DCM/MeOH from 100:0 to 90:10). Collected fractions gave the title compound D78 (0.490 g) as a slightly orange thick oil.

UPLC (IPQC): rt=1.59 minutes, peak observed: 485 (M+1) C₃₀H₃₆N₂O₂Si requires 484.

### Description 79: {(1R,4S,6R)-3-[(6-methyl-2-pyridinyl)carbonyl]-3-azabicyclo[4.1.0]hept-4-yl}methanol (D79)

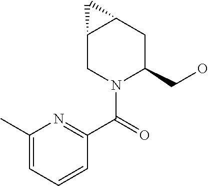

Into a 50 ml round-bottomed flask (1R,4S,6R)-4-({[(1,1-dimethylethyl)(diphenyl)silyl]oxy}methyl)-3-[(6-methyl-2-pyridinyl)carbonyl]-3-azabicyclo[4.1.0]heptane D78 (0.49 g) was dissolved in THF and the resulting solution cooled at 0° C. To this solution TBAF 1M in THF (1.112 ml, 1.112 mmol) was added dropwise, the ice-bath was removed and the reaction was left under stirring at room temperature for 2 hours. Volatiles were removed under reduced pressure and the crude oil charged into a separatory funnel containing a saturated NaHCO₃ aqueous solution (100 ml) and it was extracted with DCM (4×50 ml). The collected organic phases were dried (Na₂SO₄) and solvent was removed under reduced pressure to give an oil. This material was purified by column chromatography on silica gel (flash master, 50 g Si cartridge, eluting with DCM/MeOH from 100:0 to 90:10). Collected fractions gave the title compound D79 (0.23 g) as colorless thick oil.

UPLC (IPQC): rt=0.59 minutes, peak observed: 247 (M+1) C₁₄H₁₈N₂O₂ requires 246.

### Description 80: methyl 3-chloro-6-methyl-2-pyridinecarboxylate (D80)

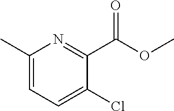

HCl 6M solution in water (14.37 ml, 86 mmol) was added to methyl 3-amino-6-methyl-2-pyridinecarboxylate D26 (2.47 g) and the resulting pale yellow mixture was sequentially diluted with water (15 ml) and chilled at 0° C. (internal temperature). A solution of sodium nitrite (1.538 g, 22.30 mmol) in water (4 ml) was dropped into the mixture over 1 minute.

After this addition the mixture was stirred at 0° C. for 30 minutes, then dropped into a suspension of copper (I) chloride (1.471 g, 14.86 mmol) in water (4 ml) over 1 minute. The mixture was left under stirring for 1 hour: during this period the temperature passed from 0° C. to 5° C.

EtOAc was then added to the stirred mixture.

Water and EtOAc were added and the mixture was poured into a separator funnel. The water phase was extracted with EtOAc. All the organic phases were joined together, dried over Na₂SO₄ and evaporated at reduced pressure, obtaining a crude that was purified in two times by column chromatography (silica 100 g column, gradient elution from Cy to Cy/EtOAc 7:3 in 30 minutes, flow rate 60 ml/min) to give the title compound D80 (1.5 gr) as an oil.

¹H NMR (400 MHz, CHLOROFORM-d) δ ppm 2.59 (s, 3H) 4.00 (s, 3H) 7.23 (d, 1H) 7.68 (d, 1H)

### Description 81: 3-chloro-6-methyl-2-pyridinecarboxylic acid lithium salt (D81)

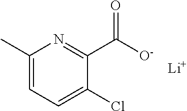

Methyl 3-chloro-6-methyl-2-pyridinecarboxylate D80 (200 mg) was dissolved in Ethanol (5 ml) into a capped vial, then a solution of lithium hydroxide (38.7 mg, 1.616 mmol) in water (2 ml) was added in one portion.

The mixture was then stirred at room temperature for 2 hours: complete conversion.

The solvent was evaporated at reduced pressure obtaining the title compound D81 (214 mg) as white solid.

¹H NMR (400 MHz, DMSO-d₆) δ ppm 2.37 (s, 3H) 7.02 (d, 1H) 7.58 (d, 1H)

### Description 82: (1R,4S,6R)-3-[(3-chloro-6-methyl-2-pyridinyl)carbonyl]-4-{[(1,1-dimethylethyl)(diphenyl)silyl]oxy}methyl)-3-azabicyclo[4.1.0]heptane (D82)

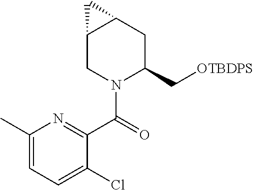

Into a 50 ml round-bottomed flask, 3-chloro-6-methyl-2-pyridinecarboxylic acid lithium salt D81 (270 mg) was added and dissolved in DCM (20 ml). To this solution DIPEA (1.433 ml, 8.21 mmol) and TBTU (0.483 g, 1.504 mmol) were added and the resulting mixture was stirred at room temperature for 30 minutes. Then a DCM solution (5 ml) of (1R,4S,6R)-4-({[(1,1-dimethylethyl)(diphenyl)silyl]oxy}methyl)-3-azabicyclo[4.1.0]heptane D10 (0.5 g) was added and the resulting mixture left under stirring at room temperature for 14 hours. Additional 3-chloro-6-methyl-2-pyridinecarboxylic acid lithium salt D81 (0.270 g) and TBTU (0.483 g, 1.504 mmol) were added and the mixture left under stirring at room temperature for additional 5 hours. The reaction mixture was transferred into a separatory funnel containing brine (50 ml) and it was extracted with DCM (4×25 ml). The collected organic phases were dried (Na₂SO₄) and evaporated under reduced pressure to give an oil. This material was purified by column chromatography on silica gel (flash master, 50 g Si cartridge, eluting with DCM/MeOH from 100:0 to 90:10). Collected fractions gave the title compound D82 (0.72 g) as a slightly yellow oil.

UPLC (IPQC): rt1=1.60 minutes and rt2=1.65 minutes (rotamers present) peak observed: 519 (M+1) C₃₀H₃₅ClN₂O₂Si requires 518.

### Description 83: {(1R,4S,6R)-3-[(3-chloro-6-methyl-2-pyridinyl)carbonyl]-3-azabicyclo[4.1.0]hept-4-yl}methanol (D83)

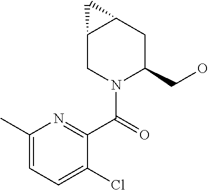

Into a 25 ml round-bottomed flask (1R,4S,6R)-3-[(3-chloro-6-methyl-2-pyridinyl)carbonyl]-4-({[(1,1-dimethylethyl)(diphenyl)silyl]oxy}methyl)-3-azabicyclo[4.1.0]heptane D82 (0.72 g) was dissolved in THF (5 ml) and the resulting solution cooled at 0° C. To this solution TBAF 1M solution in THF (1.068 ml, 1.068 mmol) was added dropwise, the ice-bath was removed and the reaction was left under stirring at room temperature for 1.5 hours. Volatiles were removed under reduced pressure and the crude oil poured into a separatory funnel containing a saturated NaHCO₃ aqueous solution (100 ml) and it was extracted with DCM (4×50 ml). The collected organic phases were dried (Na₂SO₄) and solvent was removed under reduced pressure to give an oil. This material was purified by column chromatography on silica gel (flash master, 50 g Si cartridge, eluting with DCM/MeOH from 100:0 to 90:10). Collected fractions gave the title compound D83 (0.258 g) as colorless thick oil.

UPLC (IPQC): rt1=0.67 minutes and rt2=0.71 minutes (rotamers present) peak observed: 281 (M+1) C₁₄H₁₇ClN₂O₂ requires 280.

### Description 84: 2,3-dimethylpyrazine 1-oxide (D84)

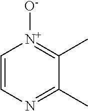

2,3-dimethylpyrazine (3.98 ml, 37.0 mmol) and MCPBA (6.38 g, 37.0 mmol) were dissolved in DCM (170 ml) and stirred at 23° C. After 42 hours triphenylphosphine (4.2 g, 16.01 mmol) was added to reduce any unreacted peracid and the mixture was stirred for 2 hours. The solvent was removed under reduced pressure and the solid obtained was purified by silica gel chromatography (SNAP KP-Sil 340 g; eluted with EtOAc/MeOH, 2 CV 100% EtOAc, 5 CV from 100% EtOAc to 90:10, 5 CV 90:10). Evaporated fractions gave the title compound D84 (3.5 g) as white solid.

UPLC (IPQC): rt=0.32 minutes, peak observed: 125 (M+1) C₆H₈N₂O requires 124.

### Description 85: 5-chloro-2,3-dimethylpyrazine (D85)

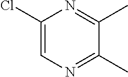

2,3-dimethylpyrazine 1-oxide D84 (3.5 g) was suspended in POCl₃ (26.3 ml, 282 mmol) and refluxed at 110° C. for 1 hour.

The reaction mixture was poured into a 1 l flask with ice and the pH value was carefully brought to ca. 8 with solid KOH; the aqueous phase was extracted with EtOAc (4×100 ml), the organic layers were collected together, dried (Na₂SO₄), filtered and evaporated under reduced pressure to give a dark oil.

It was purified by silica gel chromatography (SNAP KP-Sil 100 g eluted with Cy/EtOAc 5 CV from 100% to 70:30, 5 CV 70:30).

Evaporated fractions gave a yellowish oil, resulted to be the title compound D85 (860 mg).

UPLC (IPQC): rt=0.70 minutes, peak observed: 143 (M+1) C₆H₇ClN₂ requires 142.

### Description 86: 2,3-dimethyl-5-[(phenylmethyl)oxy]pyrazine (D86)

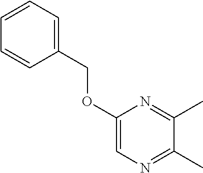

Potassium tert-butoxide (413 mg, 3.68 mmol) was added to a solution of 5-chloro-2,3-dimethylpyrazine D85 (350 mg) and benzyl alcohol (0.638 ml, 6.14 mmol) in 1,4-Dioxane (12 ml). The resulting yellow suspension was stirred at 98° C. for 20 minutes and then the temperature was allowed to reach 23° C. Water (5 ml) and EtOAc (20 ml) were added, the aqueous phase was extracted with EtOAc (3×10 ml) and the collected organic layers washed with brine (2×5 ml), dried over Na₂SO₄, filtered and evaporated under reduced pressure to give a yellow oil.

This was purified by column chromatography on silica gel (SNAP KP-Sil 50 g; eluted with Cy/EtOAc 90:10) and an orange solid was obtained. It resulted to be not pure and it was further purified by silica gel chromatography (SNAP KP-Sil; eluted with n-hexane/Et₂O 90:10). Evaporated fractions gave the title compound D86 as yellow solid (175 mg).

UPLC (IPQC): rt=1.10 minutes, peak observed: 215 (M+1) C₁₃H₁₄N₂O requires 214.

### Description 87: 5,6-dimethyl-2-pyrazinol (D87)

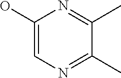

2,3-dimethyl-5-[(phenylmethyl)oxy]pyrazine D86 (175 mg) was dissolved in MeOH (8 ml) and Pd/C (8.69 mg, 0.082 mmol) was added. The mixture was stirred under H₂ atmosphere at 1 atm. After 1.5 hours the reaction was complete, the suspension was filtered and the organic solvent evaporated under reduced pressure to give an orange semisolid.

This was triturated with toluene (3×5 ml), the organic solvent was removed by suction and evaporated to give the title compound D87 (175 mg) as yellow solid.

UPLC (IPQC): rt=0.34 minutes, peak observed: 125 (M+1) C₆H₈N₂O requires 124.

### Description 88: (1R,4S,6R)-4-(2-{[5-(trifluoromethyl)-2-pyridinyl]oxy}ethyl)-3-azabicyclo[4.1.0]heptane (D88)

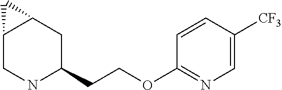

Di-tert-butyl azodicarboxylate (210 mg, 0.869 mmol) was added to a solution of 1,1-dimethylethyl (1R,4S,6R)-4-(2-hydroxyethyl)-3-azabicyclo[4.1.0]heptane-3-carboxylate D71 (100 mg), 5-(trifluoromethyl)-2(1H)-pyridinone (106 mg, 0.651 mmol) and tri-n-butylphosphine (0.214 ml, 0.869 mmol) in THF (5 ml) at 35° C. and the resulting mixture was stirred at 50° C. for 2 hours. Then the volatiles were removed to give the crude, which was purified by flash chromatography on silica (50 g column, gradient elution from Cy to Cy/EtOAc 90:10 in 4 CV then Cy/EtOAc 90:10 for 3 CV) to give desired compound as Boc-derivative.

This was dissolved in DCM and TFA (0.335 ml, 4.34 mmol) was added; the reaction mixture was stirred at room temperature for 18 hours, then the solvent was removed to give the crude which was purified two times by SCX 5 g column (DCM, MeOH and NH₃ 2 M in MeOH to elute). The ammoniacal solvent was removed to give a crude, that was further purified by flash chromatography on silica (25 g column, gradient elution from DCM to DCM/MeOH 70:30 then Cy/EtOAc 90:10 for 3 CV, flow rate 40 ml/min) to give a first batch of the title compound D88 (22 mg) and a second batch of the title compound D88 (63 mg).

UPLC (IPQC): rt=0.69 minutes, peak observed: 287 (M+1) C₁₄H₁₇F₃N₂O requires 286

### Description 89: 1,1-dimethylethyl (1R,4S,6R)-4-[2-(3-pyridinyloxy)ethyl]-3-azabicyclo[4.1.0]heptane-3-carboxylate (D89)

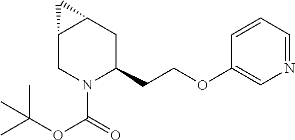

In a 25 ml round bottomed flask 1,1-dimethylethyl (1R,4S,6R)-4-(2-hydroxyethyl)-3-azabicyclo[4.1.0]heptane-3-carboxylate D71 (0.050 g) was added and dissolved in THF (3 ml). To this solution tri-n-butylphosphine (0.102 ml, 0.414 mmol) and 3-hydroxy-pyridine (0.030 g, 0.311 mmol) were added and the resulting solution was warmed to 40° C. To this solution di-tert-butyl azodicarboxylate (0.095 g, 0.414 mmol) was added and the resulting reaction mixture left under stirring at 40° C. for 1 hour. Volatiles were removed under reduced pressure, and the crude oil was purified by column chromatography on silica gel (flash master, 50 g Si cartridge, eluting with DCM/MeOH from 100:0 to 90:10). Collected fractions gave the title compound D89 (250 mg) as yellowish oil. The purity was estimated to be roughly 50% by ¹H-NMR analysis. This material was used in the next step without further purification.

UPLC (IPQC): rt=0.85 minutes, peak observed: 319 (M+1) C₁₈H₂₆N₂O₃ requires 318.

### Description 90: (1R,4S,6R)-4-[2-(3-pyridinyloxy)ethyl]-3-azabicyclo[4.1.0]heptane (D90)

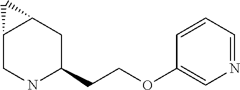

Into a 10 ml round bottomed flask 1,1-dimethylethyl (1R,4S,6R)-4-[2-(3-pyridinyloxy)ethyl]-3-azabicyclo[4.1.0]heptane-3-carboxylate D89 (0.250 g) was added and dissolved in DCM (3 ml). TFA (2 ml) was added and the resulting reaction mixture was left under stirring at room temperature for 2 hours, then reaction mixture was flashed trough a SCX column (10 g) with MeOH and NH₃ 2M in MeOH. Collected fractions gave a crude yellowish oil. This material was purified by column chromatography on silica gel (flash master, 50 g NH cartridge, eluting with DCM/MeOH from 100:0 to 80:20). Collected fractions gave the title compound D90 (27 mg) as a colorless oil.

UPLC (IPQC): rt=0.28 minutes, peak observed: 219 (M+1) C₁₃H₁₈N₂O requires 219.

### Description 91: 1,1-dimethylethyl (1R,4S,6R)-4-(2-{[(1,1-dimethylethyl)(diphenyl)silyl]oxy}ethyl)-3-azabicyclo[4.1.0]heptane-3-carboxylate (D91)

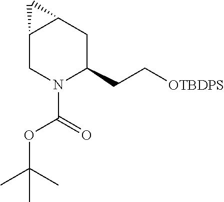

To a solution of 1,1-dimethylethyl (1R,4S,6R)-4-(2-hydroxyethyl)-3-azabicyclo[4.1.0]heptane-3-carboxylate D71 (2 g) in dry DMF (30 ml) under nitrogen, imidazole (2.82 g, 41.4 mmol) and chloro(1,1-dimethylethyl)diphenylsilane (2.73 g, 9.95 mmol) were added and the reaction stirred at room temperature overnight. EtOAc (200 ml) was added followed by water (200 ml), the organic layer was separated, dried over sodium sulphate, filtered and dried to give a crude that was purified on Si flash chromatography (SNAP 100 g+50 g column, Cy 100% to Cy/EtOAc 90:10 as eluent). The combined fractions were collected and dried under reduced pressure to give the title compound D91 (3.2 g) as a colorless oil.

UPLC (IPQC): rt=1.76 minutes, peak observed: 480 (M+1) C₂₉H₄₁NO₃Si requires 479.

### Description 92: (1R,4S,6R)-4-(2-{[(1,1-dimethylethyl)(diphenyl)silyl]oxy}ethyl)-3-azabicyclo[4.1.0]heptane (D92)

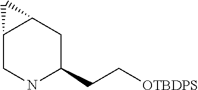

To a solution of 1,1-dimethylethyl (1R,4S,6R)-4-(2-{[(1,1-dimethylethyl)(diphenyl)silyl]oxy}ethyl)-3-azabicyclo[4.1.0]heptane-3-carboxylate D91 (3.2 g) in dry DCM (10 ml) under nitrogen, TFA (5 ml) was added and the resulting solution was stirred at room temperature for 2 hours. The mixture was dried under vacuum and the residue purified on SCX (50 g column, conditioned with MeOH and eluted with DCM then MeOH, then NH₃ 1M sol in MeOH. The combined fractions were dried to give the title compound D92 (1.8 g) as colorless oil.

UPLC (IPQC): rt=1.07 minutes, peak observed: 380 (M+1) C₂₄H₃₂NOSi requires 379.

### Description 93: (1R,4S,6R)-4-(2-{[(1,1-dimethylethyl)(diphenyl)silyl]oxy}ethyl)-3-{[6-methyl-3-(2-pyrimidinyl)-2-pyridinyl]carbonyl}-3-azabicyclo[4.1.0]heptane (D93)

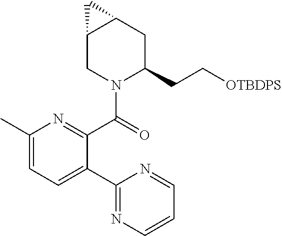

To a solution of (1R,4S,6R)-4-(2-{[(1,1-dimethylethyl)(diphenyl)silyl]oxy}ethyl)-3-azabicyclo[4.1.0]heptanes D92 (1.8 g), 6-methyl-3-(2-pyrimidinyl)-2-pyridinecarboxylic acid D59 (2.355 g) and DIPEA (2.484 ml, 14.23 mmol) in DCM (50 ml), TBTU (2.284 g, 7.11 mmol) was added and the reaction was stirred under N₂ at room temperature for 2.5 hours.

The reaction was quenched with NaHCO₃ saturated solution (30 ml), the two phases were separated and the organic one was dried over a phase separator, then it was concentrated under vacuum to obtain 6.5 g of a dark red oil.

This was purified by SNAP 110 g NH cartridge (eluting mixture Cy/EtOAc 1:1, 10 CV) to obtain a first batch of the title compound D93 (1.73 g) as yellow oil and a second batch of the title compound D93 (0.445 g) as yellow foam.

UPLC (IPQC): rt1=1.61 minutes and rt2=1.65 minutes (rotamers present) peak observed: 577 (M+1) C₃₅H₄₀N₄O₂Si requires 576.

### Description 94: 2-((1R,4S,6R)-3-{[6-methyl-3-(2-pyrimidinyl)-2-pyridinyl]carbonyl}-3-azabicyclo[4.1.0]hept-4-yl)ethanol (D94)

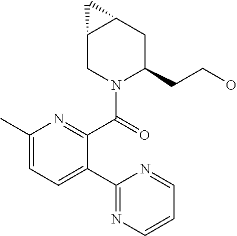

To a pale orange solution of (1R,4S,6R)-4-(2-{[(1,1-dimethylethyl)(diphenyl)silyl]oxy}ethyl)-3-{[6-methyl-3-(2-pyrimidinyl)-2-pyridinyl]carbonyl}-3-azabicyclo[4.1.0]heptane D93 (2.17 g) in THF (40 ml) at room temperature, TBAF (3.76 ml, 3.76 mmol) was added (the solution became light blue, then light green and finally turned back to yellow) and the reaction mixture was stirred at room temperature for 2 hours. The solvent was removed under vacuum to obtain 3.6 g of crude yellow oil.

This was purified by SNAP 110 g NH column (eluting mixture Cy/EtOAc from 8:2 to 0:10) and then by SNAP 100 g Si column (eluting mixture EtOAc/MeOH 95:5) to obtain a first batch of the title compound D94 (0.522 g) as whitenish gum.

During the process, there was a leak in the column: solvent was recovered and evaporated to obtain a crude. The column was then washed with MeOH 100% (200 ml) and the fractions collected were put together with the crude to obtain a yellow oil, 1.77 g.

This oil was loaded on a SNAP 50 g silica cartridge (eluted with DCM/MeOH) to obtain a second batch of the title compound D94 (0.25 g) as whitenish foam.

UPLC (IPQC): rt: 0.68 minutes and 0.73 minutes (rotamers present) peak observed: 339 (M+1), C₁₉H₂₂N₄O₂ requires 338.

### Description 95: (1R,4S,6R)-4-{[(1,1-dimethylethyl)(diphenyl)silyl]oxy}methyl)-3-{[5-methyl-2-(2-pyrimidinyl)phenyl]carbonyl}-3-azabicyclo[4.1.0]heptane (D95)

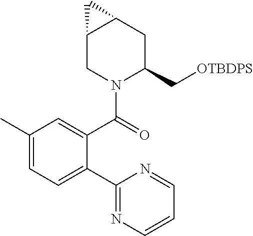

To a solution of 5-methyl-2-(2-pyrimidinyl)benzoic acid (145 mg, 0.677 mmol) and (1R,4S,6R)-4-({[(1,1-dimethylethyl)(diphenyl)silyl]oxy}methyl)-3-azabicyclo[4.1.0]heptane D10 (247 mg) in DCM (5 ml) at room temperature, DIPEA (0.296 ml, 1.692 mmol) and T₃P (1292 mg, 2.031 mmol) were added. The solution was heated to 40° C. for 2 days than water was added, mixture was extracted with DCM; organic phase was washed with NaOH 1M and then brine. DCM was dried and solvent removed to give a crude. This was added to a silica gel (50 g) column and was eluted with Cy/EtOAc 0 to 100% to give title compound D95 (127 mg) as orange oil.

UPLC (Acid GEN_QC): rt1=1.17 minutes and rt2=1.22 minutes (rotamers present) peak observed: 562 (M+1) C₃₅H₃₉N₃O₂Si requires 561.

### Description 96: ((1R,4S,6R)-3-{[5-methyl-2-(2-pyrimidinyl)phenyl]carbonyl}-3-azabicyclo[4.1.0]hept-4-yl)methanol (D96)

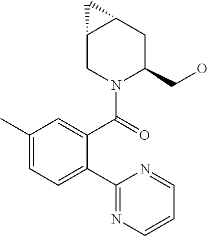

To a solution of (1R,4S,6R)-4-({[(1,1-dimethylethyl)(diphenyl)silyl]oxy}methyl)-3-{[5-methyl-2-(2-pyrimidinyl)phenyl]carbonyl}-3-azabicyclo[4.1.0]heptane D95 (127 mg) in THF (1 ml) at 20° C., TBAF (0.249 ml, 0.249 mmol) was added. After 3.5 hours NaHCO₃ saturated solution was added and the mixture extracted with DCM; the organic phase was dried and solvent removed to give a crude, that was added to a silica gel column (10 g) and was eluted with DCM/MeOH 80:20, to give the title compound D96 (56 mg) as yellow oil.

UPLC (Acid GEN_QC: rt1=0.57 minutes and rt2=0.61 minutes (rotamers present) peak observed: 324 (M+1) C₁₉H₂₁N₃O₂ requires 323.

### EXAMPLES

### Example 1

### (1R,4S,6R)-3-{[6-Methyl-3-(propyloxy)-2-pyridinyl]carbonyl}-4-({[5-(trifluoromethyl)-2-pyridinyl]oxy}methyl)-3-azabicyclo[4.1.0]heptane hydrochloride (E1)

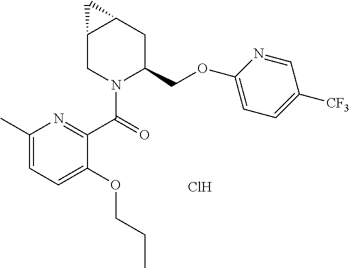

A solution of ((1R,4S,6R)-3-{[6-methyl-3-(propyloxy)-2-pyridinyl]carbonyl}-3-azabicyclo[4.1.0]hept-4-yl)methanol D14 (0.102 g) in THF (1 ml) was cooled at 0° C., NaH (60% w/w dispersion in mineral oil, 0.016 g, 0.402 mmol) was added and the mixture stirred at room temperature for 30 minutes. Then a solution of 2-chloro-5-(trifluoromethyl)pyridine (0.076 g, 0.419 mmol) in THF (1 ml) was added and the solution was gently warmed at 65-70° C. for 2 hours. The reaction was quenched with water and extracted with DCM. All the combined organic phases were dried over Na₂SO₄ and evaporated to dryness to give a crude orange oil (0.160 g) which was purified by flash chromatography on silica gel (SNAP 10 g column, eluting with DCM to DCM/MeOH 80:20) to afford the free base of the title compound (1R,4S,6R)-3-{[6-methyl-3-(propyloxy)-2-pyridinyl]carbonyl}-4-({[5-(trifluoromethyl)-2-pyridinyl]oxy}methyl)-3-azabicyclo[4.1.0]heptane (0.124 g).

UPLC (Basic GEN_QC: rt=0.99 minutes, peak observed: 450 (M+1). C₂₃H₂₆F₃N₃O₃ requires 449.0.

¹H-NMR (500 MHz, DMSO-d₆) δ ppm: 8.39-8.29 (m, 1H), 8.08-8.03 (dd, 1H), 7.35-7.28 (d, 1H), 7.12-7.06 (d, 1H), 7.00-6.95 (d, 1H), 4.62-4.00 (m, 3H), 3.99-3.84 (m, 2H), 3.72-3.61 (m, 1H), 3.37-3.33 (m, 1H), 2.17-2.09 (s, 3H), 1.89-1.78 (m, 2H), 1.71-1.52 (m, 2 H), 1.16-0.82 (m, 5H), 0.76-0.64 (m, 1H), 0.25-0.18 (m, 1H).

The remaining (1R,4S,6R)-3-{[6-methyl-3-(propyloxy)-2-pyridinyl]carbonyl}-4-({[5-(trifluoromethyl)-2-pyridinyl]oxy}methyl)-3-azabicyclo[4.1.0]heptane was dissolved in DCM (2.5 ml) and 1 M HCl in Et₂O (0.020 ml, 0.670 mmol) was added and then stirred for 1 hour. The volatiles were removed under reduced pressure and the solid obtained was triturated with Et₂O (2.5 ml) which was removed by suction. The solid was dried under reduced pressure to afford the title compound E1 (0.14 g) like yellow foam. UPLC (Basic GEN_QC): rt=0.99 minutes, peak observed: 450 (M-HCl+1) C₂₃H₂₆F₃N₃O₃.HCl requires 486.

### Example 2

### (1R,4S,6R)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane (E2)

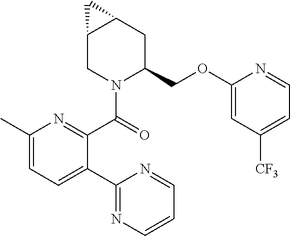

((1S,4S,6S)-3-{[6-methyl-3-(2-pyrimidinyl)-2-pyridinyl]carbonyl}-3-azabicyclo[4.1.0]hept-4-yl)methanol D46 (20 mg) was dissolved in DMF (2 ml). NaH 60% wt. (2.466 mg, 0.062 mmol) was added and the mixture was shaken for 30 minutes at room temperature.

2-fluoro-4-(trifluoromethyl)pyridine was added and the resulting mixture was shaken at 50° C. for 4 hours. Solvent was then removed under vacuum, and the crude was taken up with DCM and straightforward purified with Biotage SP1 (10 g SNAP KP—NH column, with a gradient of Cy/EtOAc). The title compound E2 (13 mg) was recovered as colourless solid.

UPLC (Acid GEN_QC_SS): rt1=0.94 minutes and rt2=0.96 minutes (rotamers present), peak observed: 470 (M+1). C₂₄H₂₂F₃N₅O₂ requires 469. ¹H-NMR (500 MHz, DMSO-d₆) δ ppm: 8.91-8.81 (, 2H), 8.51-8.47 (d, 1H), 8.47-8.43 (d, 1H), 7.51-7.41 (m, 2H), 7.41-7.36 (m, 1H), 7.28-7.22 (m, 1H), 4.72-4.44 (m, 3H), 3.61-3.52 (dd, 1H), 3.29-3.21 (dd, 1H), 2.40-2.33 (s, 3H), 2.30-2.17 (m, 1H), 1.86-1.76 (m, 1H), 1.10-0.89 (m, 2H), 0.67-0.56 (m, 1H), 0.55-0.43 (m, 1H).

The following compounds were prepared using a similar procedure to that described for Example 1 and Example 2. Each compound was obtained by reacting ((1R,4S,6R)-3-{[heteroaryl-carbonyl}-3-azabicyclo[4.1.0]hept-4-yl)methanol with the appropriate halo derivative. This is provided merely for assistance to the skilled chemist. The starting material may not necessarily have been prepared from the batch referred to.

| No. | Reactants | Characterising data |
|---|---|---|
| 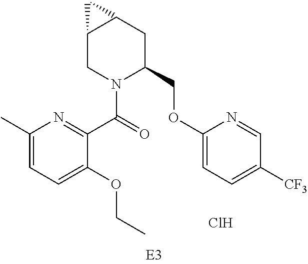 | D18 and 2-chloro-5- (trifluoromethyl) pyridine | (1R,4S,6R)-3-{[3-(ethyloxy)-6-methyl-2- pyridinyl]carbonyl}-4-({[5-(trifluoromethyl)-2- pyridinyl]oxy}methyl)-3-azabicyclo[4.1.0]heptane UPLC (Basic GEN_QC): rt = 0.95 minutes, peak observed: 436 (M + 1). C₂₂H₂₄F₃N₃O₃ requires 435. ¹H- NMR (400 MHz, DMSO-d₆) δ ppm: 8.20-8.06 (m, 1 H), 7.88-7.81 (dd, 1 H), 7.12-7.06 (d, 1 H), 6.91-6.85 (d, 1 H), 6.79-6.73 (d, 1 H), 4.38-4.26 (m, 1 H), 4.28- 4.11 (m, 2 H), 3.88-3.65 (m, 2 H), 3.51-3.39 (m, 1 H), 3.18-3.02 (m, 1 H), 1.97-1.85 (s, 3 H), 1.71-1.50 (m, 2 H), 1.11-0.93 (t, 3 H), 0.96-0.75 (m, 2 H), 0.54-0.41 (m, 1 H), 0.04-0.03 (m, 1 H). (1R,4S,6R)-3-{[3-(ethyloxy)-6-methyl-2- pyridinyl]carbonyl}-4-({[5-(trifluoromethyl)-2- pyridinyl]oxy}methyl)-3-azabicyclo[4.1.0]heptane hydrochloride HPLC (walk-up): rt = 5.34 minutes. C₂₂H₂₄F₃N₃O₃•HCl requires 471. ¹H NMR (400 MHz, DMSO-d₆) δ ppm 8.28-8.38 (m, 1 H), 7.99-8.14 (m, 1 H), 6.93-7.57 (m, 3 H), 4.32-4.61 (m, 3 H), 3.73- 4.20 (m, 3 H), 3.12-3.42 (m, 1 H), 2.12-2.18 (m, 3 H), 1.77-1.92 (m, 2 H), 0.82-1.38 (m, 5 H), 0.51- 0.75 (m, 1 H), 0.15-0.36 (m, 1 H). |
| 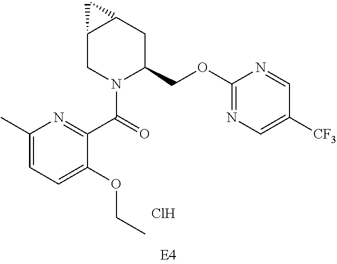 | D18 and 2-chloro-5- (trifluoromethyl) pyrimidine | (1R,4S,6R)-3-{[3-(ethyloxy)-6-methyl-2- pyridinyl]carbonyl}-4-({[5-(trifluoromethyl)-2- pyrimidinyl]oxy}methyl)-3- azabicyclo[4.1.0]heptane HPLC: rt = 0.85 minutes and rt = 0.86 minutes (rotamers present), peak observed: 437 (M + 1). C₂₁H₂₃F₃N₄O₃ requires 436. ¹H-NMR (400 MHz, DMSO-d₆) δ ppm: 9.32-9.23 (s, 2 H), 7.87-7.56 (m, 2 H), 5.29-4.92 (m, 3 H), 4.70-4.42 (m, 2 H), 4.39-3.76 (m, 1 H), 3.01-2.92 (s, 3 H), 2.5-2.38 (m, 2 H), 1.93- 1.82 (m, 3 H), 1.76-1.44 (m, 2 H), 1.35-1.25 (m, 1 H), 0.88-0.82 (m, 1 H). (1R,4S,6R)-3-{[3-(ethyloxy)-6-methyl-2- pyridinyl]carbonyl}-4-({[5-(trifluoromethyl)-2- pyrimidinyl]oxy}methyl)-3- azabicyclo[4.1.0]heptane hydrochloride HPLC (walk-up): rt = 4.65 minutes. C₂₁H₂₃F₃N₄O₃•HCl requires 472. ¹H NMR (400 MHz, DMSO-d₆) δ ppm 8.90-9.00 (m, 2 H), 7.01-7.56 (m, 2 H), 4.36-4.66 (m, 3 H), 3.28-4.26 (m, 4 H), 2.13 (s, 3 H), 1.73-1.94 (m, 2 H), 0.77-1.42 (m, 5 H), 0.51-0.75 (m, 1 H), 0.19-0.34 (m, 1 H). |
| 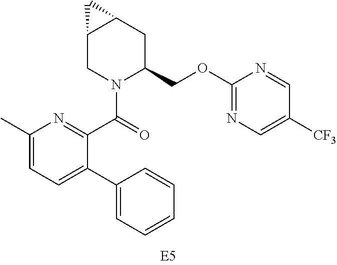 | D44 and 2-chloro-5- (trifluoromethyl) pyrimidine | (1R,4S,6R)-3-[(6-methyl-3-phenyl-2- pyridinyl)carbonyl]-4-({[5-(trifluoromethyl)-2- pyrimidinyl]oxy}methyl)-3- azabicyclo[4.1.0]heptane UPLC (Acid GEN_QC): rt1 = 0.79 minutes and rt2 = 0.81 minutes (rotamers present), peak observed: 469 (M + 1). C₂₅H₂₃F₃N₄O₂ requires 468. ¹H NMR (500 MHz, CDCl₃) δ ppm 8.78 (s, 2 H), 7.61-7.65 (m, 1 H), 7.33-7.54 (m, 5 H), 7.25 (d, 1 H), 4.69-4.84 (m, 1 H), 4.34-4.55 (m, 2 H), 3.24-3.43 (m, 1 H), 2.98- 3.09 (m, 1 H), 2.63 (s, 3 H), 2.18-2.28 (m, 1 H), 1.55- 1.75 (m, 1 H), 0.84-0.95 (m, 1 H), 0.70-0.77 (m, 1 H), 0.38-0.49 (m, 1 H), −0.30-0.01 (m, 1 H). |
| 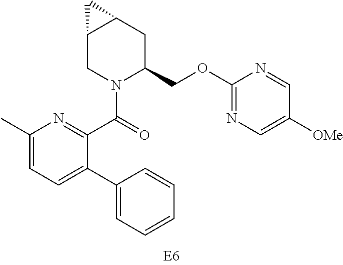 | D44 and 2-chloro-5- (methyloxy)pyrimidine | (1R,4S,6R)-4-({[5-(methyloxy)-2- pyrimidinyl]oxy}methyl)-3-[(6-methyl-3-phenyl-2- pyridinyl)carbonyl]-3-azabicyclo[4.1.0]heptane UPLC (Acid GEN_QC): rt1 = 0.70 minutes and rt2 = 0.73 minutes (rotamers present), peak observed: 431 (M + 1). C₂₅H₂₆N₄O₃ requires 430. ¹H NMR (500 MHz, CDCl₃) δ ppm 8.12 (s, 2 H), 7.57-7.66 (m, 2 H), 7.31- 7.55 (m, 5 H), 7.22 (d, 1 H), 4.47 (d, 1 H), 4.22-4.39 (m, 2 H), 3.89 (s, 3 H), 3.47-3.58 (m, 1 H), 3.26- 3.36 (m, 1 H), 2.54 (s, 3 H), 1.80-1.94 (m, 1 H), 0.84- 0.92 (m, 1 H), 0.73-0.85 (m, 1 H), 0.63-0.76 (m, 1 H), 0.52-0.61 (m, 1 H), −0.36--0.08 (m, 1 H) |
| 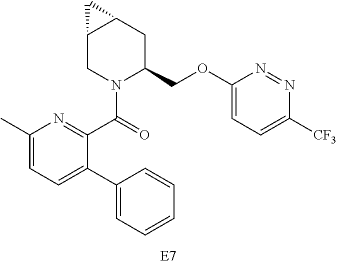 | D44 and 3-chloro-6- (trifluoromethyl) pyridazine | (1R,4S,6R)-3-[(6-methyl-3-phenyl-2- pyridinyl)carbonyl]-4-({[6-(trifluoromethyl)-3- pyridazinyl]oxy}methyl)-3-azabicyclo[4.1.0]heptane UPLC (Acid GEN_QC): rt1 = 0.83 minutes and rt2 = 0.85 minutes (rotamers present), peak observed: 469 (M + 1). C₂₅H₂₃F₃N₄O₂ requires 468. ¹H NMR (500 MHz, CDCl₃) δ ppm 7.67-7.73 (m, 1 H), 7.57 (d, 1 H), 7.30-7.54 (m, 5 H), 7.24 (d, 1 H), 7.05-7.15 (m, 1 H), 4.46-4.69 (m, 3 H), 3.60-3.69 (m, 1 H), 3.28- 3.35 (m, 1 H), 2.61 (s, 3 H), 1.51-1.59 (m, 1 H), 0.84- 0.92 (m, 1 H), 0.66-0.81 (m, 2 H), 0.48-0.56 (m, 1 H), −0.70--0.49 (m, 1 H). |
| 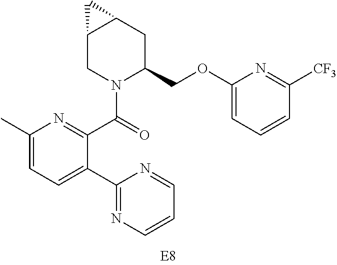 | D46 and 2-fluoro-6- (trifluoromethyl) pyridine | (1R,4S,6R)-3-{[6-methyl-3-(2-pyrimidinyl)-2- pyridinyl]carbonyl}-4-({[6-(trifluoromethyl)-2- pyridinyl]oxy}methyl)-3-azabicyclo[4.1.0]heptane UPLC (Basic GEN_QC): rt1 = 0.91 minutes and rt2 = 0.97 minutes (rotamers present), peak observed: 470 (M + 1). C₂₄H₂₂F₃N₅O₂ requires 469. ¹H-NMR (500 MHz, DMSO-d₆) δ ppm: 8.93-8.85 (m, 2H), 8.51-8.45 (d, 1H), 8.05-7.98 (t, 1H), 7.55-7.51 (d, 1H), 7.48-7.36 (m, 2H), 7.26-7.19 (d, 1H), 4.70-4.38 (m, 3H), 3.60- 3.51 (d, 1H), 3.27-3.13 (d, 1H), 2.58-2.51 (s, 3H), 2.34-2.23 (m, 1H), 1.87-1.77 (m, 1H), 1.18-0.96 (m, 2H), 0.67-0.59 (m, 1H), 0.50-0.43 (m, 1H) |
| 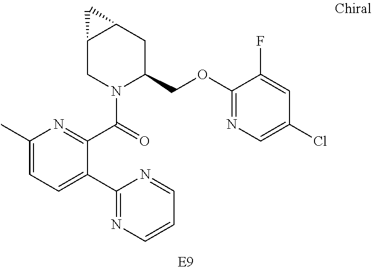 | D46 and 5-chloro-2,3- difluoropyridine | (1R,4S,6R)-4-{[(5-chloro-3-fluoro-2- pyridinyl)oxy]methyl}-3-{[6-methyl-3-(2- pyrimidinyl)-2-pyridinyl]carbonyl}-3- azabicyclo[4.1.0]heptane UPLC (Basic GEN_QC): rt1 = 0.92 minutes, and rt2 = 0.95 minutes (rotamers present), peak observed: 454 (M + 1). C₂₃H₂₁ClFN₅O₂ requires 453. ¹H NMR (500 MHz, DMSO-d₆) δ ppm 0.16-0.23 (m, 1 H) 0.27-0.35 (m, 1 H) 0.62-0.80 (m, 2 H) 1.48- 1.62 (m, 1 H) 1.89-1.97 (m, 1 H) 2.23 (s, 3 H) 2.94 (dd, 1 H) 3.26 (dd, 1 H) 4.16-4.41 (m, 3 H) 7.12- 7.15 (m, 1 H) 7.17 (t, 1 H) 7.75 (dd, 1 H) 7.83 (d, 1 H) 8.15 (d, 1 H) 8.50-8.59 (m, 2 H) |
| 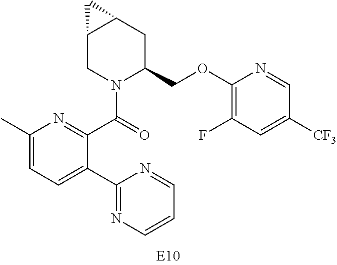 | D46 and 2,3-difluoro- 5- (trifluoromethyl) pyridine | (1R,4S,6R)-4-({[3-fluoro-5-(trifluoromethyl)-2- pyridinyl]oxy}methyl)-3-{[6-methyl-3-(2- pyrimidinyl)-2-pyridinyl]carbonyl}-3- azabicyclo[4.1.0]heptane UPLC (Basic GEN_QC): rt1 = 0.94 minutes and rt2 = 0.98 minutes (rotamers present), peak observed: 488 (M + 1). C₂₄H₂₁F₄N₅O₂ requires 487. ¹H NMR (500 MHz, DMSO-d₆) δ ppm 0.14-0.24 (m, 1 H) 0.26-0.36 (m, 1 H) 0.61-0.79 (m, 2 H) 1.48- 1.62 (m, 1 H) 1.87-1.98 (m, 1 H) 2.04 (s, 3 H) 2.89- 2.97 (m, 1 H) 3.27 (dd, 1 H) 4.24-4.53 (m, 3 H) 7.11- 7.20 (m, 2 H) 7.82-7.86 (m, 1 H) 7.95 (d, 1 H) 8.14 (d, 1 H) 8.53-8.59 (m, 2 H) |
| 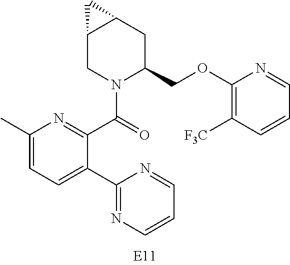 | D46 and 2-fluoro-3- (trifluoromethyl) pyridine | (1R,4S,6R)-3-{[6-methyl-3-(2-pyrimidinyl)-2- pyridinyl]carbonyl}-4-({[3-(trifluoromethyl)-2- pyridinyl]oxy}methyl)-3-azabicyclo[4.1.0]heptane UPLC (Acid GEN_QC_SS): rt1 = 0.90 minutes and rt2 = 0.94 minutes (rotamers present), peak observed: 470 (M + 1): C₂₄H₂₂F₃N₅O₂ requires 469. 1H-NMR (500 MHz, DMSO-d₆) δ ppm: 8.92-8.73 (m, 2H), 8.56- 8.39 (m, 2H), 8.20-8.08 (m, 1H), 7.50-7.39 (m, 2H), 7.27-7.19 (m, 1H), 4.72-4.52 (m, 3H), 3.68-3.48 (m, 1H), 3.29-3.21 (m, 1H), 2.55-2.53 (s, 3H), 2.34-2.23 (m, 1H), 1.89-1.73 (m, 1H), 1.09-0.93 (m, 2H), 0.64- 0.56 (m, 1H), 0.57-0.50 (m, 1H) |
| 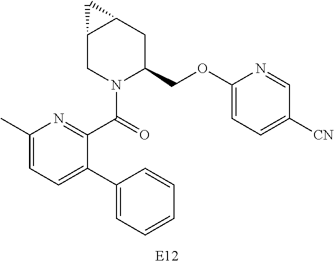 | D44 and 6-bromo-3- pyridinecarbonitrile | 6-[({(1R,4S,6R)-3-[(6-methyl-3-phenyl-2- pyridinyl)carbonyl]-3-azabicyclo[4.1.0]hept-4- yl}methyl)oxy]-3-pyridinecarbonitrile UPLC (Acid FINAL_QC): rt1 = 0.77 minutes and rt2 = 0.79 minutes (rotamers present), peak observed 425 (M + 1), C₂₆H₂₇N₃O₃ required 424. ¹H NMR (500 MHz, CDCl₃) δ ppm 0.14-0.41 (m, 1 H) 1.00-1.09 (m, 1 H) 1.17-1.52 (m, 3 H) 2.06-2.18 (m, 1 H) 3.12 (s, 3 H) 3.56 (d, 1 H) 3.97-4.09 (m, 1 H) 4.67-4.95 (m, 2 H) 5.03 (d, 1 H) 7.36 (d, 1 H) 7.69 (d, 1 H) 7.73-8.04 (m, 5 H) 8.13 (d, 1 H) 8.24-8.36 (m, 1 H) 8.96-9.04 (m, 1 H) |
| 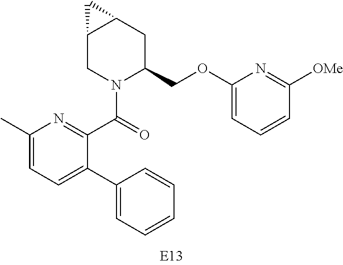 | D44 and 2-bromo-6- (methyloxy)pyridine | (1R,4S,6R)-4-({[6-(methyloxy)-2- pyridinyl]oxy}methyl)-3-[(6-methyl-3-phenyl-2- pyridinyl)carbonyl]-3-azabicyclo[4.1.0]heptane UPLC (Acid FINAL_QC): rt1 = 0.83 minutes and rt2 = 0.88 minutes (rotamers present), peak observed 430 (M + 1), C₂₆H₂₇N₃O₃ required 429. ¹H NMR (500 MHz, CDCl₃) δ ppm −0.24 (br. s., 0 H) 0.42-0.53 (m, 0 H) 0.66-0.95 (m, 2 H) 1.59-1.68 (m, 2 H) 2.56 (s, 3 H) 3.19-3.28 (m, 1 H) 3.42-3.51 (m, 1 H) 3.86 (s, 3 H) 3.91-4.33 (m, 2 H) 4.47 (d, 1 H) 6.14-6.27 (m, 2 H) 7.09 (d, 1 H) 7.25-7.46 (m, 6 H) 7.47-7.60 (m, 1 H) |
| 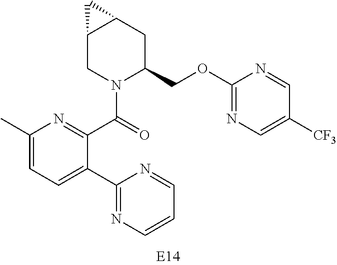 | D46 and 2-chloro-5- (trifluoromethyl) pyrimidine | (1R,4S,6R)-3-{[6-methyl-3-(2-pyrimidinyl)-2- pyridinyl]carbonyl}-4-({[5-(trifluoromethyl)-2- pyrimidinyl]oxy}methyl)-3- azabicyclo[4.1.0]heptane UPLC (Acid GEN_QC_SS): rt1 = 1.00 minutes and rt2 = 1.02 minutes (rotamers present), peak observed: 470 (M + 1). C₂₃H₂₁Cl₂N₅O₂ requires 439. ¹H NMR (400 MHz, CDCl₃) d ppm 0.54-0.64 (m, 1 H) 0.65- 0.76 (m, 1 H) 0.80-1.37 (m, 2 H) 1.87-1.97 (m, 1 H) 2.39-2.48 (m, 1 H) 2.63 (s, 3 H) 3.34 (dd, 1 H) 3.61- 3.71 (m, 1 H) 4.42-4.93 (m, 2 H) 4.93-5.04 (m, 1 H) 7.15-7.31 (m, 2 H) 8.57 (d, 1 H) 8.80 (s, 2 H) 8.83 (d, 2 H) |
| 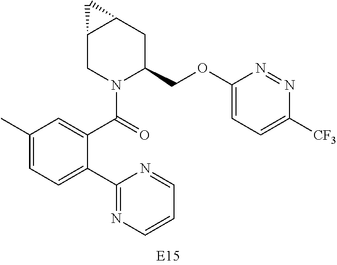 | D96 and 3-chloro-6- (trifluoromethyl) pyridazine | (1R,4S,6R)-3-{[5-methyl-2-(2- pyrimidinyl)phenyl]carbonyl}-4-({[6- (trifluoromethyl)-3-pyridazinyl]oxy}methyl)-3- azabicyclo[4.1.0]heptane UPLC (Basic GEN_QC): rt1 = 0.90 minutes and rt2 = 0.93 minutes (rotamers present) peak observed: 470 (M + 1) C₂₄H₂₂F₃N₅O₂ requires: 469. ¹H NMR (500 MHz, DMSO-d₆) δ ppm 0.10-0.19 (m, 1 H) 0.65-0.77 (m, 1 H) 0.85-1.01 (m, 1 H) 0.99- 1.12 (m, 1 H) 1.19-1.31 (m, 1 H) 1.65-1.79 (m, 1 H) 2.17 (s, 3 H) 3.29-3.41 (m, 1 H) 3.83-3.96 (m, 1 H) 4.47 (d, 1 H) 4.55-4.90 (m, 2 H) 7.00-7.06 (m, 1 H) 7.21 (d, 1 H) 7.42 (t, 1 H) 7.58-7.63 (m, 1 H) 7.95 (d, 1 H) 8.14-8.24 (m, 1 H) 8.79-8.90 (m, 2 H) |
| 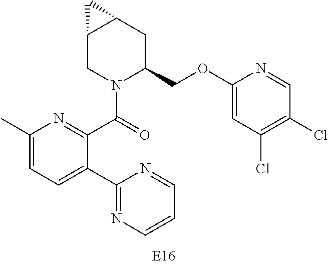 | D46 and D77 | (1R,4S,6R)-4-{[(4,5-dichloro-2- pyridinyl)oxy]methyl}-3-{[6-methyl-3-(2- pyrimidinyl)-2-pyridinyl]carbonyl}-3- azabicyclo[4.1.0]heptane UPLC (IPQC): rt = 1.11 minutes peak observed: 471 (M + 1) C₂₃H₂₁Cl₂N₅O₂ requires 470. |
| 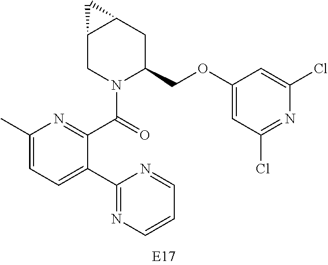 | D46 and 2,4,6- trichloropyridine | (1R,4S,6R)-4-{[(2,6-dichloro-4- pyridinyl)oxy]methyl}-3-{[6-methyl-3-(2- pyrimidinyl)-2-pyridinyl]carbonyl}-3- azabicyclo[4.1.0]heptane ¹H NMR (500 MHz, DMSO-d₆) δ ppm 0.14-0.22 (m, 1 H) 0.27-0.35 (m, 1 H) 0.61-0.78 (m, 2 H) 1.43- 1.52 (m, 1 H) 1.84-1.94 (m, 1 H) 2.24 (s, 3 H) 2.89 (dd, 1 H) 3.26 (dd, 1 H) 3.95-4.32 (m, 3 H) 7.04 (s, 2 H) 7.09-7.21 (m, 2 H) 8.16 (d, 1 H) 8.51-8.59 (m, 2 H) |
| 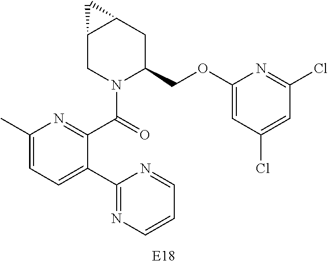 | D46 and 2,4,6- trichloropyridine | (1R,4S,6R)-4-{[(4,6-dichloro-2- pyridinyl)oxy]methyl}-3-{[6-methyl-3-(2- pyrimidinyl)-2-pyridinyl]carbonyl}-3- azabicyclo[4.1.0]heptane UPLC (Acid GEN_QC_SS): rt1 = 1.00 minutes and 1.04 minutes (rotamers present) peak observed: 470 (M + 1) C₂₃H₂₁Cl₂N₅O₂ requires 469. ¹H NMR (500 MHz, DMSO-d₆) δ ppm 0.43-0.50 (m, 1 H) 0.58-0.66 (m, 1 H) 0.93-1.07 (m, 2 H) 1.76- 1.83 (m, 1 H) 2.20-2.27 (m, 1 H) 2.54 (s, 3 H) 3.22 (dd, 1 H) 3.55 (dd, 1 H) 4.38-4.47 (m, 1 H) 4.56- 4.63 (m, 2 H) 7.11-7.13 (m, 1 H) 7.37-7.39 (m, 1 H) 7.43-7.50 (m, 2 H) 8.47 (d, 1 H) 8.92 (d, 2 H) |
| 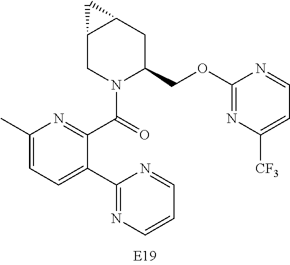 | D46 and 2-chloro-4- (trifluoromethyl) pyrimidine | (1R,4S,6R)-3-{[6-methyl-3-(2-pyrimidinyl)-2- pyridinyl]carbonyl}-4-({[4-(trifluoromethyl)-2- pyrimidinyl]oxy}methyl)-3- azabicyclo[4.1.0]heptane UPLC (Acid GEN_QC_SS): rt1 = 0.81 minutes and rt2 = 0.86 minutes (rotamers present) peak observed: 471 (M + 1) C₂₃H₂₁F₃N₆O₂ requires 470. ¹H NMR (500 MHz, DMSO-d₆) δ ppm 0.19 (d, 1 H) 0.21-0.35 (m, 1 H) 0.55-0.64 (m, 1 H) 0.66-0.76 (m, 1 H) 1.44-1.53 (m, 1 H) 1.90 (dd, 1 H) 2.01 (br. s., 3 H) 2.78-2.96 (m, 1 H) 3.23 (dd, 1 H) 4.08-4.27 (m, 1 H) 4.26-4.51 (m, 2 H) 7.05-7.14 (m, 2 H) 7.34 (d, 1 H) 8.12 (d, 1 H) 8.51-8.62 (m, 2 H) 8.69 (d, 1 H) |
| 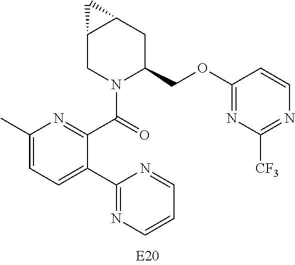 | D46 and 4-chloro-2- (trifluoromethyl) pyrimidine | (1R,4S,6R)-3-{[6-methyl-3-(2-pyrimidinyl)-2- pyridinyl]carbonyl}-4-({[2-(trifluoromethyl)-4- pyrimidinyl]oxy}methyl)-3- azabicyclo[4.1.0]heptane UPLC (Acid GEN_QC_SS): rt1 = 0.82 minutes and rt2 = 0.88 minutes (rotamers present) peak observed: 471 (M + 1) C₂₃H₂₁F₃N₆O₂ requires 470. ¹H NMR (500 MHz, DMSO-d₆) δ ppm 0.45-0.54 (m, 1 H) 0.59-0.68 (m, 1 H) 0.92-1.11 (m, 2 H) 1.77- 1.86 (m, 1 H) 2.22-2.32 (m, 1 H) 2.50 (s, 3 H) 3.22 (d, 1 H) 3.58 (dd, 1 H) 4.53-4.61 (m, 1 H) 4.61-4.70 (m, 1 H) 4.71-4.82 (m, 1 H) 7.32 (d, 1 H) 7.42-7.49 (m, 2 H) 8.47 (d, 1 H) 8.76-8.81 (m, 1 H) 8.89 (d, 2H) |
| 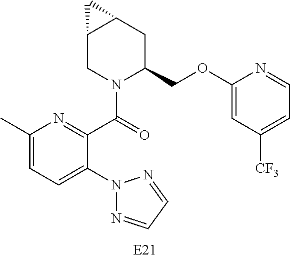 | D63 and 2-fluoro-4- (trifluoromethyl) pyridine | (1R,4S,6R)-3-{[6-methyl-3-(2H-1,2,3-triazol-2-yl)-2- pyridinyl]carbonyl}-4-({[4-(trifluoromethyl)-2- pyridinyl]oxy}methyl)-3-azabicyclo[4.1.0]heptane UPLC (Acid GEN_QC): rt1 = 0.82 minutes and rt2 = 0.83 minutes (rotamers present) peak observed: 459 (M + 1) C₂₂H₂₁F₃N₆O₂ requires 458. ¹H NMR (500 MHz, DMSO-d₆) δ ppm 0.40-0.50 (m, 1 H) 0.56-0.65 (m, 1 H) 0.92-1.08 (m, 2 H) 1.74- 1.91 (m, 1 H) 2.12-2.27 (m, 1 H) 2.54 (s, 3 H) 3.25- 3.32 (m, 1 H) 3.57 (dd, 1 H) 4.40-4.59 (m, 2 H) 4.60- 4.70 (m, 1 H) 7.08-7.24 (m, 1 H) 7.32 (d, 1 H) 7.48 (d, 1 H) 8.07 (s, 2 H) 8.19 (d, 1 H) 8.47 (d, 1 H) |
| 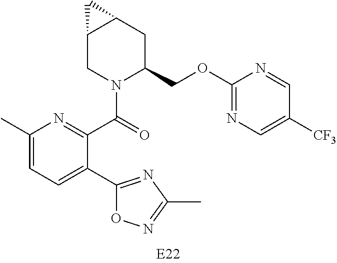 | D61 and 2-chloro-5- (trifluoromethyl) pyrimidine | (1R,4S,6R)-3-{[6-methyl-3-(3-methyl-1,2,4- oxadiazol-5-yl)-2-pyridinyl]carbonyl}-4-({[5- (trifluoromethyl)-2-pyrimidinyl]oxy}methyl)-3- azabicyclo[4.1.0]heptane UPLC (Acid GEN_QC_SS): rt1 = 0.87 minutes and 0.90 minutes (rotamers present) peak observed: 475 (M + 1) C₂₂H₂₁F₃N₆O₃ requires 474. ¹H NMR (500 MHz, CHLOROFORM-d) δ ppm 0.49- 0.59 (m, 1 H) 0.66-0.74 (m, 1 H) 0.96-1.03 (m, 1 H) 1.05-1.14 (m, 1 H) 1.90-2.01 (m, 1 H) 2.40-2.52 (m, 4 H) 2.66 (s, 3 H) 3.22-3.31 (m, 1 H) 3.66-3.73 (m, 1 H) 4.63-4.89 (m, 2 H) 4.90-5.01 (m, 1 H) 7.28- 7.35 (m, 1 H) 8.22-8.32 (m, 1 H) 8.79 (s, 2 H) |
| 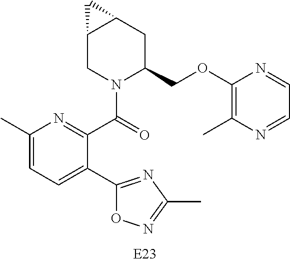 | D61 and 2-chloro-3- methylpyrazine | (1R,4S,6R)-3-{[6-methyl-3-(3-methyl-1,2,4- oxadiazol-5-yl)-2-pyridinyl]carbonyl}-4-{[(3- methyl-2-pyrazinyl)oxy]methyl}-3- azabicyclo[4.1.0]heptane UPLC (Acid GEN_QC_SS): rt1 = 0.76 minutes and rt2 = 0.80 minutes (rotamers present) peak observed: 421 (M + 1) C₂₃H₂₄N₆O₃ requires 420. ¹H NMR (500 MHz, DMSO-d₆) δ ppm 0.28-0.42 (m, 1 H) 0.53-0.65 (m, 1 H) 0.92-1.19 (m, 2 H) 1.82- 1.91 (m, 1 H) 2.17-2.25 (m, 1 H) 2.37 (s, 3 H) 2.47 (s, 3 H) 2.56 (s, 3 H) 3.16-3.25 (m, 1 H) 3.53-3.59 (m, 1 H) 4.32-4.65 (m, 2 H) 4.72-4.80 (m, 1 H) 7.55 (d, 1 H) 7.93-8.10 (m, 2 H) 8.38 (d, 1 H) |
| 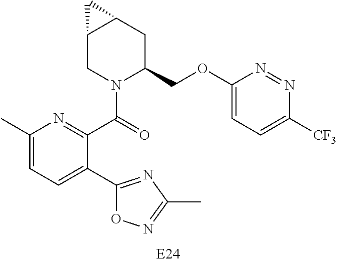 | D61 and 3-chloro-6- (trifluoromethyl) pyridazine | (1R,4S,6R)-3-{[6-methyl-3-(3-methyl-1,2,4- oxadiazol-5-yl)-2-pyridinyl]carbonyl}-4-({[6- (trifluoromethyl)-3-pyridazinyl]oxy}methyl)-3- azabicyclo[4.1.0]heptane UPLC (Acid GEN_QC): rt1 = 0.86 minutes and rt2 = 0.90 minutes (rotamers present) peak observed: 475 (M + 1) C₂₂H₂₁F₃N₆O₃ requires 474. ¹H NMR (500 MHz, DMSO-d₆) δ ppm 0.39-0.44 (m, 0 H) 0.73-0.80 (m, 0 H) 0.94-1.21 (m, 2 H) 1.69- 2.29 (m, 2 H) 2.38 (s, 3 H) 2.57 (s, 3 H) 3.21 (dd, 1 H) 3.58 (dd, 1 H) 4.60-4.89 (m, 3 H) 7.35-7.62 (m, 2 H) 8.16 (d, 1 H) 8.33 (d, 1 H) |
| 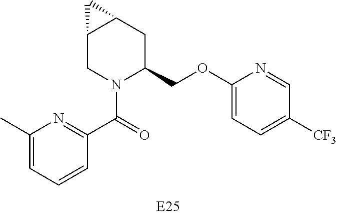 | D79 and 2-fluoro-5- (trifluoromethyl) pyridine | (1R,4S,6R)-3-[(6-methyl-2-pyridinyl)carbonyl]-4- ({[5-(trifluoromethyl)-2-pyridinyl]oxy}methyl)-3- azabicyclo[4.1.0]heptane UPLC (IPQC): rt = 1.15 minutes, peak observed: 392 (M + 1) C₂₀H₂₀F₃N₃O₂ requires 391 ¹H NMR (400 MHz, DMSO-d₆) δ ppm 0.07-0.15 (m, 1 H) 0.71-0.81 (m, 1 H) 0.94-1.09 (m, 1 H) 1.10-1.21 (m, 1 H) 1.80-1.92 (m, 2 H) 2.37 (s, 3 H) 3.27-3.37 (m, 1 H) 4.08-4.17 (m, 1 H) 4.30-4.61 (m, 3 H) 6.91-7.01 (m, 2 H) 7.20 (d, 1 H) 7.54 (t, 1 H) 8.03 (dd, 1 H) 8.27-8.34 (m, 1 H) |
| 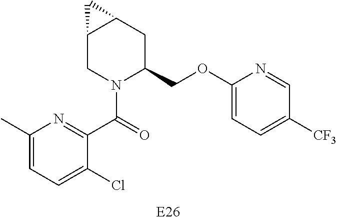 | D83 and 2-fluoro-5- (trifluoromethyl) pyridine | (1R,4S,6R)-3-[(3-chloro-6-methyl-2- pyridinyl)carbonyl]-4-({[5-(trifluoromethyl)-2- pyridinyl]oxy}methyl)-3-azabicyclo[4.1.0]heptane UPLC (IPQC): rt = 1.23 minutes, peak observed: 426 (M + 1) C₂₀H₁₉ClF₃N₃O₂ requires 425 ¹H NMR (500 MHz, DMSO-d₆) δ ppm 0.10-0.23 (m, 1 H) 0.70-0.80 (m, 1 H) 1.00-1.09 (m, 1 H) 1.11-1.19 (m, 1 H) 1.79-1.89 (m, 2 H) 2.28 (s, 3 H) 3.37 (dd, 1 H) 3.60-3.69 (m, 1 H) 4.34-4.66 (m, 3 H) 7.01 (t, 1 H) 7.27 (d, 1 H) 7.78 (d, 1 H) 8.02- 8.13 (m, 1 H) 8.36-8.41 (m, 1 H) |
| 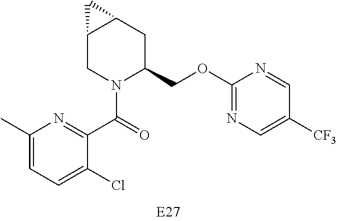 | D83 and 2-chloro-5- (trifluoromethyl) pyrimidine | (1R,4S,6R)-3-[(3-chloro-6-methyl-2- pyridinyl)carbonyl]-4-({[5-(trifluoromethyl)-2- pyrimidinyl]oxy}methyl)-3- azabicyclo[4.1.0]heptane UPLC (IPQC): rt1 = 1.06 minutes and rt2 = 1.17 minutes (rotamers present, peak observed: 427 (M + 1) C₁₉H₁₈ClF₃N₄O₂ requires 426 ¹H NMR (500 MHz, DMSO-d₆) δ ppm 0.25-0.31 (m, 1 H) 0.62 (m, 1 H) 0.94-1.11 (m, 2 H) 1.81- 1.92 (m, 1 H) 2.09-2.18 (m, 1 H) 2.45 (s, 3 H) 3.12 (dd, 1 H) 3.60 (dd, 1 H) 4.65 (m, 2 H) 4.75-4.85 (m, 1 H) 7.34 (d, 1 H) 7.87 (d, 1 H) 8.93-9.16 (m, 2 H) |
| 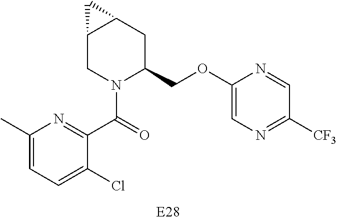 | D83 and 2-chloro-5- (trifluoromethyl) pyrazine | (1R,4S,6R)-3-[(3-chloro-6-methyl-2- pyridinyl)carbonyl]-4-({[5-(trifluoromethyl)-2- pyrazinyl]oxy}methyl)-3-azabicyclo[4.1.0]heptane UPLC (IPQC): rt1 = 1.15 minutes and rt2 = 1.16 minutes (rotamers present) peak observed: 427 (M + 1) C₁₉H₁₈ClF₃N₄O₂ requires 426. ¹H NMR (500 MHz, DMSO-d₆) δ ppm 0.27 (q, 1 H) 0.63 (m, 1 H) 0.93-1.11 (m, 2 H) 1.81-1.93 (m, 1 H) 2.11 (m, 1 H) 2.45 (s, 3 H) 3.10 (dd, 1 H) 3.59 (dd, 1 H) 4.66 (m, 2 H) 4.78-4.79 (m, 1 H) 7.34 (d, 1 H) 7.88 (d, 1 H) 8.47 (s, 1 H) 8.78 (s, 1 H) |
| 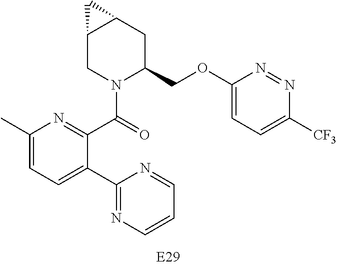 | D46 and 3-chloro-6- (trifluoromethyl) pyridazine | (1R,4S,6R)-3-{[6-methyl-3-(2-pyrimidinyl)-2- pyridinyl]carbonyl}-4-({[6-(trifluoromethyl)-3- pyridazinyl]oxy}methyl)-3-azabicyclo[4.1.0]heptane C₂₃H₂₁F₃N₆O₂ requires 470. ¹H NMR (400 MHz, DMSO-d₆) δ ppm 0.13-0.24 (m, 1 H) 0.25-0.36 (m, 1 H) 0.61-0.79 (m, 2 H) 1.47-1.56 (m, 1 H) 1.91-2.00 (m, 1 H) 2.21 (s, 3 H) 2.89-2.96 (m, 1 H) 3.27 (dd, 1 H) 4.33-4.52 (m, 3 H) 7.13 (d, 1 H) 7.16 (t, 1 H) 7.22 (d, 1 H) 7.86 (d, 1 H) 8.15 (d, 1 H) 8.55 (d, 1 H) |
| 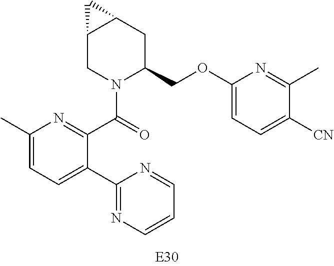 | D46 and 6-fluoro-2- methyl-3- pyridinecarbonitrile | 2-methyl-6-{[((1R,4S,6R)-3-{[6-methyl-3-(2- pyrimidinyl)-2-pyridinyl]carbonyl}-3- azabicyclo[4.1.0]hept-4-yl)methyl]oxy}-3- pyridinecarbonitrile C₂₅H₂₄N₆O₂ requires 440 ¹H NMR (500 MHz, DMSO-d₆) δ ppm 0.44-0.53 (m, 1 H) 0.57-0.68 (m, 1 H) 0.93-1.01 (m, 1 H) 1.01-1.07 (m, 1 H) 1.77-1.84 (m, 1 H) 2.20-2.31 (m, 1 H) 2.54 (s, 3 H) 2.63 (s, 3 H) 3.23 (dd, 1 H) 3.56 (dd, 1 H) 4.42-4.70 (m, 3 H) 6.89 (d, 1 H) 7.43- 7.46 (m, 1 H) 7.49 (t, 1 H) 8.10 (d, 1 H) 8.46 (d, 1 H) 8.89 (d, 2 H) |
| 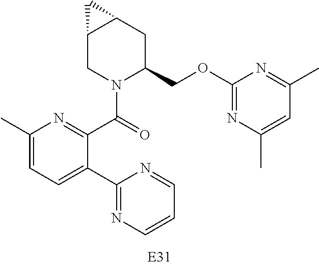 | D46 and 2-chloro-4,6- dimethylpyrimidine | (1R,4S,6R)-4-{[(4,6-dimethyl-2- pyrimidinyl)oxy]methyl}-3-{[6-methyl-3-(2- pyrimidinyl)-2-pyridinyl ]carbonyl}-3- azabicyclo[4.1.0]heptane C₂₄H₂₆N₆O₂ requires 430. ¹H NMR (500 MHz, DMSO-d₆) δ ppm 0.50-0.55 (m, 1 H) 0.59-0.66 (m, 1 H) 0.92-0.99 (m, 1 H) 0.99-1.07 (m, 1 H) 1.76-1.84 (m, 1 H) 2.21-2.28 (m, 1 H) 2.39 (s, 6 H) 2.55 (s, 3 H) 3.25 (dd, 1 H) 3.56 (dd, 1 H) 4.38-4.45 (m, 1 H) 4.55-4.66 (m, 2 H) 6.93 (s, 1 H) 7.42-7.47 (m, 1 H) 7.49 (t, 1 H) 8.48 (d, 1 H) 8.95 (d, 2 H) |

### Example 32

### 2-[({(1R,4S,6R)-3-[(6-methyl-3-phenyl-2-pyridinyl)carbonyl]-3-azabicyclo[4.1.0]hept-4-yl}methyl)oxy]-1,3-benzoxazole (E32)

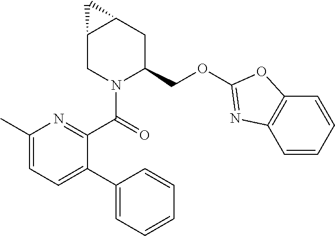

To a solution of {(1R,4S,6R)-3-[(6-methyl-3-phenyl-2-pyridinyl)carbonyl]-3-azabicyclo[4.1.0]hept-4-yl}methanol D44 (30 mg) in THF (1 ml) at 20° C., sodium hydride (2.456 mg, 0.102 mmol) was added. After 5 minutes 2-chloro-1,3-benzoxazole (10.62 μl, 0.093 mmol) was added and the mixture heated, in a microwave oven, to 100° C. for 1 hour. The mixture was further heated to 120° C. for 1 hour then DMF (1 ml) was added followed by sodium hydride (2.456 mg, 0.102 mmol) and after 10 minutes the mixture was heated to 150° C. for additional 1 hour. NaHCO₃ saturated solution was added and the mixture extracted with DCM. Organic phase was loaded and purified by SCX (2 g) using MeOH and 2M NH₃ in MeOH. Ammoniacal phase was evaporated in vacuum to give a brown oil, which was purified by Fraction Lynx (GEN_ACID method) to give title compound E32 (1.7 mg) as colourless oil.

UPLC (Acid GEN_QC: rt=0.87 minutes, peak observed: 440 (M+1) C₂₇H₂₅N₃O₃ requires 439.

¹H NMR (400 MHz, CHLOROFORM-d) δ ppm 0.10 (q, 1H) 0.69 (td, 1H) 0.82-0.95 (m, 1H) 0.95-1.08 (m, 1H) 1.61-1.74 (m, 2H) 2.59 (s, 3H) 3.51 (dd, 1H) 3.90 (dd, 1H) 4.11-4.22 (m, 1H) 4.35 (qd, 2H) 6.97-7.05 (m, 1H) 7.11-7.19 (m, 1H) 7.23 (d, 1H) 7.26-7.46 (m, 7H) 7.61 (d, 1H)

### Example 33

### (1R,4S,6R)-4-{[(5-fluoro-2-pyridinyl)oxy]methyl}-3-{[6-methyl-3-(2-pyrimidinyl)-2-pyridinyl]carbonyl}-3-azabicyclo[4.1.0]heptane (E33)

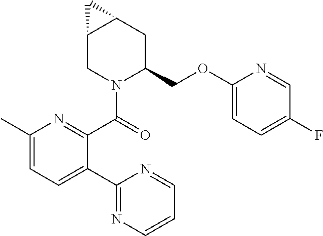

In a 8 ml screw-capped vial, ((1R,4S,6R)-3-{[6-methyl-3-(2-pyrimidinyl)-2-pyridinyl]carbonyl}-3-azabicyclo[4.1.0]hept-4-yl)methanol D46 (25 mg) was dissolved in THF (1.8 ml), to this solution 5-fluoro-2-pyridinol (13.07 mg, 0.116 mmol), PBu₃ (0.039 ml, 0.154 mmol) and 1,1′-(azodicarbonyl)dipiperidine (38.9 mg, 0.154 mmol) were added. The resulting mixture was stirred at 23° C. for 1 hour and then the solvent was removed under reduced pressure to give a yellow oil. It was purified by silica gel chromatography (SNAP KP-Sil 10 g; eluted with from 100% DCM to DCM/MeOH 98:2) and then by silica gel chromatography (SNAP KP—NH 11 g cartridge; Cy/EtOAc 1:1). The material obtained from this purification was contaminated by tributylphosphine oxide, so it was submitted to a HPLC preparative purification (Method 20 ml_ACID_GENERIC). The fractions containing the desired compound were collected and the organic solvent was removed in vacuo. The aqueous residue was extracted with DCM separated through a hydrophobic filter and evaporated to give the title compound E33 (3.6 mg).

UPLC (Basic GEN_QC: rt=0.81 minutes, peak observed: 420 (M+1) C₂₃H₂₂FN₅O₂ requires 419. ¹H NMR (500 MHz, DMSO-d₆) δ ppm 0.45-0.52 (m, 1H) 0.57-0.66 (m, 1 H) 0.91-1.09 (m, 2H) 1.76-1.84 (m, 1H) 2.18-2.26 (m, 1H) 2.55 (s, 3H) 3.25 (dd, 1H) 3.55 (dd, 1H) 4.36-4.55 (m, 2H) 4.57-4.65 (m, 1H) 6.93 (dd, 1H) 7.38-7.51 (m, 2H) 7.69-7.76 (m, 1H) 8.20 (d, 1H) 8.46 (d, 1H) 8.83-8.90 (m, 2H).

### Example 34

### (1R,4S,6R)-4-{[(4-fluoro-2-pyridinyl)oxy]methyl}-3-{[6-methyl-3-(2-pyrimidinyl)-2-pyridinyl]carbonyl}-3-azabicyclo[4.1.0]heptane (E34)

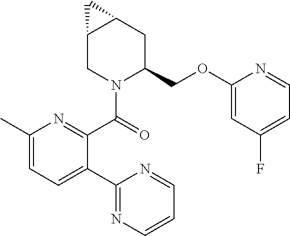

((1R,4S,6R)-3-{[6-methyl-3-(2-pyrimidinyl)-2-pyridinyl]carbonyl}-3-azabicyclo[4.1.0]hept-4-yl)methanol D46 (20 mg) and 4-fluoro-2-pyridinol (10.46 mg, 0.092 mmol) were dissolved in THF (1.5 ml) and then PBu₃ (0.031 ml, 0.123 mmol) was added. The mixture was heated to 50° C. and DEAD (28.4 mg, 0.123 mmol) was added at this temperature. After 40 min the reaction was complete, the solvent was removed under reduced pressure and the yellow oil obtained was purified by silica gel chromatography (SNAP KP-Sil 10 g; eluted with 100% DCM to DCM/MeOH 98:2). Collected and evaporated fractions gave yellowish oil which was purified by HPLC preparative purification (Method 20 ml_ACID_GENERIC). The product obtained was still contamined by tributylphosphine oxide so it was charged on a SCX 1 g column to afford a colorless oil which was left 4 hours under vacuum at 50° C. and the title compound E34 (3.6 mg) was obtained as white solid.

UPLC (Basic GEN_QC): rt=0.81 minutes, peak observed: 420 (M+1) C₂₃H₂₂FN₅O₂ requires 419. ¹H NMR (500 MHz, DMSO-d₆) δ ppm 0.45-0.53 (m, 1H) 0.56-0.65 (m, 1H) 0.89-1.09 (m, 2H) 1.74-1.84 (m, 1H) 2.14-2.28 (m, 1H) 2.55 (s, 3H) 3.19-3.31 (m, 1H) 3.56 (dd, 1H) 4.41-4.69 (m, 3H) 6.80 (dd, 1H) 6.93-7.03 (m, 1H) 7.38-7.52 (m, 2H) 8.22-8.29 (m, 1H) 8.46 (d, 1H) 8.82-8.91 (m, 2H).

### Example 35

### (1R,4S,6R)-3-{[6-methyl-3-(2-pyrimidinyl)-2-pyridinyl]carbonyl}-4-({[6-methyl-4-(trifluoromethyl)-2-pyridinyl]oxy}methyl)-3-azabicyclo[4.1.0]heptane (E35)

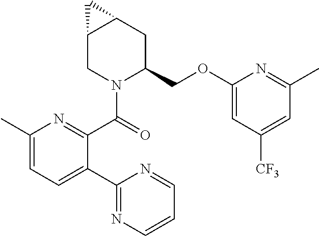

((1R,4S,6R)-3-{[6-methyl-3-(2-pyrimidinyl)-2-pyridinyl]carbonyl}-3-azabicyclo[4.1.0]hept-4-yl)methanol D46 and 6-methyl-4-(trifluoromethyl)-2-pyridinol were reacted using a similar procedure as for Example 34 to afford the title compound E35 (35 mg).

UPLC (IPQC): rt1=1.25 minutes and rt2=1.29 minutes (rotamers present), peak observed: 484 (M+1) C₂₅H₂₄F₃N₅O₂ requires 483.

¹H NMR (500 MHz, DMSO-d₆) δ ppm 0.44-0.52 (m, 1H) 0.59-0.66 (m, 1H) 0.94-1.09 (m, 2H) 1.77-1.85 (m, 1H) 2.22-2.28 (m, 1H) 2.29 (s, 3H) 2.54 (s, 3H) 3.19-3.26 (m, 1H) 3.53-3.61 (m, 1H) 4.40-4.67 (m, 3H) 7.00-7.04 (m, 1H) 7.22-7.26 (m, 1H) 7.42-7.52 (m, 2H) 8.47 (d, 1H) 8.90 (d, 2H).

### Example 36

### (1R,4S,6R)-4-{[(6-chloro-2-pyridinyl)oxy]methyl}-3-{[6-methyl-3-(2-pyrimidinyl)-2-pyridinyl]carbonyl}-3-azabicyclo[4.1.0]heptane (E36)

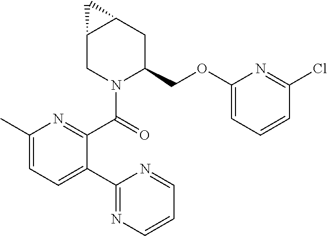

((1R,4S,6R)-3-{[6-methyl-3-(2-pyrimidinyl)-2-pyridinyl]carbonyl}-3-azabicyclo[4.1.0]hept-4-yl)methanol D46 (20.11 mg), 6-chloro-2(1H)-pyridinone (12.05 mg, 0.093 mmol), TMAD (16.01 mg, 0.093 mmol), PBu₃ (18.82 mg, 0.093 mmol) were collected and reacted in Toluene (2 ml) at room temperature for 24 hours and monitored. The reaction was further shaken at room temperature for 24 hours without any relevant change. The reaction mixture was purified with SCX 1 g column and the crude was further purified with (Biotage SP1, over a 12 g C18 column, eluting with a gradient of CH₃CN and water). The title compound E36 (12 mg) was recovered as colourless film.

UPLC (Acid GEN_QC_SS): rt1=0.88 minutes and rt2=0.94 minutes (rotamers present), peak observed: 437 (M+1) C₂₃H₂₂ClN₅O₂ requires 436.

¹H-NMR (400 MHz, DMSO-d₆) δ ppm: 8.95-8.92 (m, 2H), 8.50-8.45 (d, 1H), 7.84-7.78 (t, 1H), 7.51-7.38 (m, 2H), 7.17-7.12 (d, 1H), 6.93-6.89 (d, 1H), 4.63-4.36 (m, 3H), 3.59-3.52 (dd, 1H), 3.26-3.20 (dd, 1H), 2.57-2.53 (s, 3H), 2.34-2.20 (m, 1H), 1.86-1.76 (m, 1H), 1.11-0.88 (m, 2H), 0.67-0.58 (m, 1H), 0.51-0.42 (m, 1H).

### Example 37

### (1R,4S,6R)-4-{[(3,5-dichloro-2-pyridinyl)oxy]methyl}-3-{[6-methyl-3-(2-pyrimidinyl)-2-pyridinyl]carbonyl}-3-azabicyclo[4.1.0]heptane (E37)

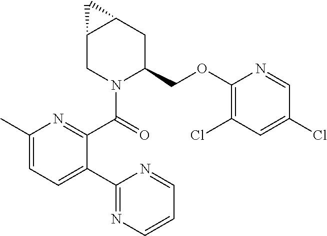

((1R,4S,6R)-3-{[6-methyl-3-(2-pyrimidinyl)-2-pyridinyl]carbonyl}-3-azabicyclo[4.1.0]hept-4-yl)methanol D46 (20.11 mg) and 3,5-dichloro-2-pyridinol (15.25 mg, 0.093 mmol) were reacted using a similar procedure as for example 36 to afford the title compound E37 (4 mg).

UPLC (Acid GEN_QC_SS): rt1=1.00 minutes and rt2=1.02 minutes (rotamers present), peak observed: 470 (M+1) C₂₃H₂₁Cl₂N₅O₂ requires 439. ¹H NMR (400 MHz, CDCl₃) d ppm 0.54-0.62 (m, 1H) 0.65-0.73 (m, 1H) 1.01-1.82 (m, 2H) 1.89-2.00 (m, 1H) 2.39-2.51 (m, 1H) 2.66 (s, 3H) 3.37 (dd, 1H) 3.66-3.75 (m, 1H) 4.45-4.80 (m, 2H) 4.92-5.02 (m, 1H) 7.19 (t, 1H) 7.29-7.32 (m, 1H) 7.69 (d, 1H) 8.07 (d, 1H) 8.58 (d, 1H) 8.79 (d, 2H)

### Example 38

### (1R,4S,6R)-4-{[(4-chloro-2-pyridinyl)oxy]methyl}-3-{[6-methyl-3-(2-pyrimidinyl)-2-pyridinyl]carbonyl}-3-azabicyclo[4.1.0]heptane (E38)

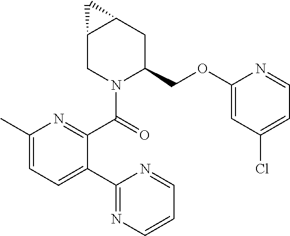

((1R,4S,6R)-3-{[6-methyl-3-(2-pyrimidinyl)-2-pyridinyl]carbonyl}-3-azabicyclo[4.1.0]hept-4-yl)methanol D46 (20.11 mg), PBu₃ (18.82 mg, 0.093 mmol), 4-chloro-2(1H)-pyridinone (12.05 mg, 0.093 mmol) were collected in a vial and dissolved in Toluene (2 ml). TMAD (16.01 mg, 0.093 mmol) was added and the resulting solution was shaken at room temperature overnight. The resulting mixture was then monitored. Starting material was still present, and no trace of product was detected. The reaction was then repeated in THF.

((1R,4S,6R)-3-{[6-methyl-3-(2-pyrimidinyl)-2-pyridinyl]carbonyl}-3-azabicyclo[4.1.0]hept-4-yl)methanol D46 (0.020 g), PBu₃ (0.019 g, 0.093 mmol), 4-chloro-2(1H)-pyridinone (0.012 g, 0.093 mmol) were collected in a vial and suspended in TMAD (16.01 mg, 0.093 mmol) and the resulting solution was shaken for 2 hours and monitored. Solvent was removed under vacuum, and the resulting crude was dissolved in 1M HCl aqueous (1 ml) and purified with Biotage SP1, over a 12 g C18 column, eluting with a gradient of water and ACN (maked up with HCOOH 0.05%) to afford the title compound E38 (10 mg).

UPLC (Acid GEN_QC_SS): rt1=0.90 minutes and rt2=0.92 minutes (rotamers present) peak observed: 436 (M+1) C₂₃H₂₂ClN₅O₂ requires 435.

¹H NMR (400 MHz, DMSO-d₆) δ ppm 0.42-0.53 (m, 1H) 0.56-0.67 (m, 1H) 0.91-1.11 (m, 2H) 1.72-1.85 (m, 1H) 2.15-2.26 (m, 1H) 2.55 (s, 3H) 3.24 (dd, 1H) 3.56 (dd, 1H) 4.35-4.63 (m, 2H) 4.57-4.68 (m, 1H) 7.04 (d, 1H) 7.15 (dd, 1H) 7.38-7.54 (m, 2H) 8.21 (d, 1H) 8.46 (d, 1H) 8.81-8.92 (m, 2H)

### Example 39

### (1R,4S,6R)-4-{[(5-chloro-2-pyridinyl)oxy]methyl}-3-{[6-methyl-3-(2-pyrimidinyl)-2-pyridinyl]carbonyl}-3-azabicyclo[4.1.0]heptane (E39)

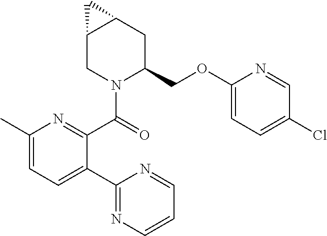

((1R,4S,6R)-3-{[6-methyl-3-(2-pyrimidinyl)-2-pyridinyl]carbonyl}-3-azabicyclo[4.1.0]hept-4-yl)methanol D46 (20.11 mg), 5-chloro-2(1H)-pyridinone (12.05 mg, 0.093 mmol), PBu₃ (18.82 mg, 0.093 mmol) were suspended in THF (2 ml). TMAD (16.01 mg, 0.093 mmol) was added and the mixture was shaken for 2 hours. Then it was monitored confirming the presence of the required product. Solvent was removed under vacuum, then it was dissolved in 1M aqueous HCl (1 ml) and purified with Biotage Sp1, over a 12 g C18 column, eluting with a gradient of water and ACN (maked up HCOOH, 0.5%). The title compound E39 was recovered as oil (10 mg).

UPLC (Acid GEN_QC_SS): rt1=0.91 minutes and rt2=0.94 minutes (rotamers present) peak observed: 436 (M+1) C₂₃H₂₂ClN₅O₂ requires 435.

¹H NMR (400 MHz, DMSO-d₆) δ ppm 0.44-0.53 (m, 1H) 0.56-0.66 (m, 1H) 0.89-1.09 (m, 2H) 1.74-1.84 (m, 1H) 2.15-2.28 (m, 1H) 2.55 (s, 3H) 3.20-3.30 (m, 1H) 3.56 (dd, 1H) 4.21-4.60 (m, 2H) 4.57-4.69 (m, 1H) 6.94 (d, 1H) 7.38-7.52 (m, 2H) 7.84 (dd, 1H) 8.25-8.30 (m, 1H) 8.43-8.50 (m, 1H) 8.82-8.91 (m, 2H)

### Example 40

### (1R,4S,6R)-4-{[(3-chloro-2-pyridinyl)oxy]methyl}-3-{[6-methyl-3-(2-pyrimidinyl)-2-pyridinyl]carbonyl}-3-azabicyclo[4.1.0]heptane (E40)

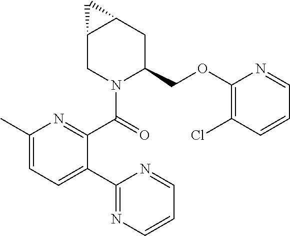

((1R,4S,6R)-3-{[6-methyl-3-(2-pyrimidinyl)-2-pyridinyl]carbonyl}-3-azabicyclo[4.1.0]hept-4-yl)methanol D46 (20.11 mg) and 3-chloro-2(1H)-pyridinone (8.03 mg, 0.062 mmol) were reacted using a similar procedure as for example 39 to afford the title compound E40 (5 mg).

UPLC (Basic GEN_QC): rt1=0.88 minutes and rt2=0.91 minutes (rotamers present) peak observed: 436 (M+1) C₂₃H₂₂ClN₅O₂ requires 435.

### Example 41

### (1R,4S,6R)-4-{[(5,6-dimethyl-2-pyrazinyl)oxy]methyl}-3-{[6-methyl-3-(2-pyrimidinyl)-2-pyridinyl]carbonyl}-3-azabicyclo[4.1.0]heptane (E41)

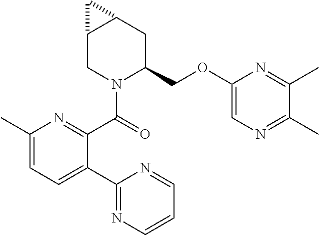

A solution of ((1R,4S,6R)-3-{[6-methyl-3-(2-pyrimidinyl)-2-pyridinyl]carbonyl}-3-azabicyclo[4.1.0]hept-4-yl)methanol D46 (89 mg), 5,6-dimethyl-2-pyrazinol (34 mg, 0.274 mmol) and tributylphosphine (0.137 ml, 0.548 mmol) in THF (6 ml) was heated to 50° C. After stirring 5 minutes at this temperature di-tert-butyl azodicarboxylate (126 mg, 0.548 mmol) was added and the resulting mixture was stirred for 3 hours at 50° C. The solvent was then removed under reduced pressure and the brown oil obtained was charged on a SCX cartridge (2 g), washed with 25 ml of MeOH and eluted with 10 ml of 2M NH₃/MeOH.

The ammoniacal fraction was evaporated under vacuum and the orange oil obtained was purified by chromatography on silica gel (SNAP KP—NH, 2×10 g; eluted with Cy/EtOAc from 100% Cy to 100% EtOAc in 5 CV, 100% EtOAc 5 CV). Evaporated fractions gave a mixture of mainly desired compound with other impurities.

This mixture was further purified by column chromatography (SNAP KP—NH; eluted with Cy/iPrOH 5 CV from 100% Cy to 95:5, 7 CV 95:5); evaporated fraction gave the title compound E41 (42 mg) as white solid.

UPLC (IPQC): rt1=0.91 minutes and rt2=0.99 minutes (rotamers present) peak observed: 431 (M+1) C₂₄H₂₆N₆O₂ requires 430.

¹H NMR (500 MHz, DMSO-d₆) δ ppm 0.45-0.52 (m, 1H) 0.58-0.66 (m, 1H) 0.93-1.08 (m, 2H) 1.76-1.83 (m, 1H) 2.21-2.28 (m, 1H) 2.41 (s, 3H) 2.44 (s, 3H) 2.54 (s, 3H) 3.24 (dd, 1H) 3.56 (dd, 1H) 4.40-4.46 (m, 1H) 4.54-4.65 (m, 2H) 7.45 (d, 1H) 7.49 (t, 1H) 8.04 (s, 1H) 8.46 (d, 1H) 8.89 (d, 2H)

### Example 42

### (1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane (E42)

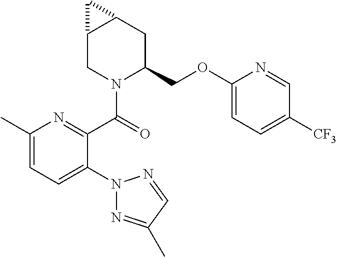

TBTU (32.4 mg, 0.101 mmol) was added to a stirred solution of 6-methyl-3-(4-methyl-2H-1,2,3-triazol-2-yl)-2-pyridinecarboxylic acid D35 (20.04 mg) and DIPEA (0.019 ml, 0.110 mmol) in DMF (1 ml) at room temperature. The mixture was stirred at room temperature for minutes then (1R,4S,6R)-4-({[5-(trifluoromethyl)-2-pyridinyl]oxy}methyl)-3-azabicyclo[4.1.0]heptane D22 (25 mg) was added. The reaction mixture was stirred at room temperature for 4 hours. The reaction was quenched with saturated NaHCO₃ solution (30 ml), and extracted with EtOAc (2×20 ml). The combined organic phases were washed with brine/water (1:1, 20 ml) and brine (20 ml), dried (Na₂SO₄) and evaporated under reduced pressure to give a pale yellow residue which was purified by flash chromatography on silica gel (Biotage KP—NH, 2× Snap-11 g column, EtOAc/Cy from 30:70 to 60:40 and then Biotage Snap 10-10 g column, EtOAc/Cy from 50:50 to 80:20) to give the title compound E42 (38 mg) as a colourless gum.

UPLC (Acid QC_POS_{—}50-800): rt1=0.84 minutes and rt2=0.88 minutes (rotamers present), peak observed: 473 (M+1) C₂₃H₂₃F₃N₆O₂ requires 472.

¹H NMR (500 MHz, DMSO-d₆) δ ppm 0.42-0.50 (m, 1H) 0.56-0.65 (m, 1H) 0.92-1.16 (m, 2H) 1.75-1.88 (m, 1H) 2.16-2.26 (m, 1H) 2.33 (s, 3H) 2.53 (s, 3H) 3.25 (d, 1H) 3.55 (d, 1H) 4.29-4.73 (m, 3H) 7.07 (d, 1H) 7.49 (d, 1H) 7.83-7.85 (m, 1H) 8.07-8.13 (m, 1H) 8.17 (d, 1H) 8.57-8.66 (m, 1H).

### Example 43

### (1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane (E43)

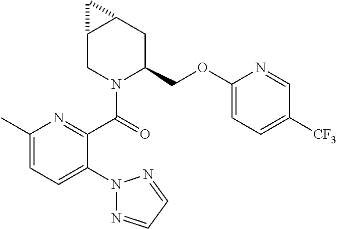

TBTU (29.4 mg, 0.092 mmol) was added to a stirred solution of 6-methyl-3-(2H-1,2,3-triazol-2-yl)-2-pyridinecarboxylic acid D33 (17 mg) and DIPEA (0.017 ml, 0.100 mmol) in DMF (1 ml) at room temperature. The mixture was stirred for 10 minutes then (1R,4S,6R)-4-({[5-(trifluoromethyl)-2-pyridinyl]oxy}methyl)-3-azabicyclo[4.1.0]heptane D22 (22.67 mg) was added. The resulting mixture was stirred for 18 hours. The reaction was quenched with saturated NaHCO₃ solution, and extracted with EtOAc. The combined organic phases were washed with brine/water (1:1, 20 ml) and brine, dried (Na₂SO₄) and evaporated under reduced pressure to give an orange residue which was purified via Biotage (30-80% EtOAc/cyclohexane; SNAP 11 g NH column) to give the title compound E43 (37 mg) of pale yellow glass.

UPLC (Basic QC_{—}50_{—}800_pos): rt1=0.94 minutes and rt2=0.96 minutes (rotamers present), peak observed: 459 (M+1) C₂₂H₂₁F₃N₆O₂ requires 458.

¹H NMR (400 MHz, DMSO-d₆) δ ppm 8.32-8.39 (m, 1H), 8.21 (d, 1H), 7.99-8.13 (m, 3 H), 7.51 (d, 1H), 7.06 (d, 1H), 4.61-4.72 (m, 1H), 4.43-4.65 (m, 2H), 4.35 (d, 1H), 3.39 (dd, 1H), 2.34 (s, 3H), 2.09-2.25 (m, 1H), 1.72-1.88 (m, 1H), 0.89-1.18 (m, 2H), 0.54-0.64 (m, 1H), 0.18-0.28 (m, 1H).

The following compounds were prepared using a similar procedure to that described for Example 42 and Example 43 (in some examples the solvent used was DCM instead of DMF). Each compound was obtained by amide coupling of (1R,4S,6R)-4-({[5-(trifluoromethyl)-2-pyridinyl]oxy}methyl)-3-azabicyclo[4.1.0]heptane D22 or (1R,4S,6R)-4-({[5-(trifluoromethyl)-2-pyrazinyl]oxy}methyl)-3-azabicyclo[4.1.0]heptane D68 with the appropriate carboxylic acid. This is provided merely for assistance to the skilled chemist.

The starting material may not necessarily have been prepared from the batch referred to.

| | Amide coupling | |
|---|---|---|
| No. | Reactants | Characterising data |
| 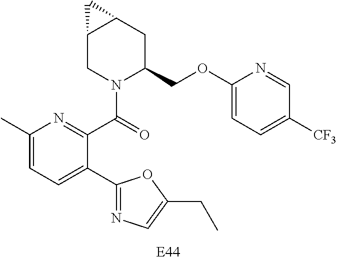 | D22 and D57 | (1R,4S,6R)-3-{[3-(5-ethyl-1,3-oxazol-2-yl)-6-methyl- 2-pyridinyl]carbonyl}-4-({[5-(trifluoromethyl)-2- pyridinyl]oxy}methyl)-3-azabicyclo[4.1.0]heptane UPLC (Basic_GEN_QC): rt1 = 1.0 minutes and rt2 = 1.4 minutes (rotamers present), peak observed: 487 (M + 1). C₂₅H₂₅F₃N₄O₃ requires 486. ¹H NMR (500 MHz, DMSO-d₆) d ppm 8.30-8.36 (m, 1 H), 7.95 (d, 1 H), 7.83 (dd, 1 H), 7.15 (d, 1 H), 6.79 (d, 1 H), 6.68- 6.71 (m, 1 H), 4.38-4.45 (m, 1 H), 4.04-4.39 (m, 2 H), 3.26 (dd, 1 H), 2.82-2.90 (m, 1 H), 2.38-2.48 (m, 2 H), 2.09 (s, 3 H), 1.90-2.00 (m, 1 H), 1.50-1.64 (m, 1 H), 0.91 (t, 3 H), 0.62-0.81 (m, 2 H), 0.43-0.50 (m, 1 H), 0.04-0.11 (m, 1 H). |
| 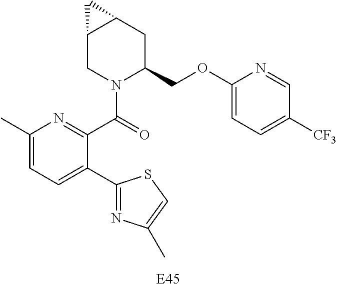 | D22 and D31 | (1R,4S,6R)-3-{[6-methyl-3-(4-methyl-1,3-thiazol-2- yl)-2-pyridinyl]carbonyl}-4-({[5-(trifluoromethyl)- 2-pyridinyl]oxy}methyl)-3-azabicyclo[4.1.0]heptane UPLC (Basic_GEN_QC): rt1 = 1.0 minutes and rt2 = 1.03 minutes (rotamers present), peak observed: 489 (M + 1). C₂₄H₂₃F₃N₄O₂S requires 488. ¹H NMR (500 MHz, DMSO-d₆) δ ppm 8.54-8.63 (m, 1 H), 7.94- 8.18 (m, 2 H), 7.29-7.49 (m, 2 H), 7.05 (d, 1 H), 4.67- 4.74 (m, 1 H), 4.24-4.67 (m, 2 H), 3.49-3.58 (m, 1 H), 3.07-3.20 (m, 1 H), 2.32-2.44 (m, 6 H), 2.11- 2.25 (m, 1 H), 1.73-1.82 (m, 1 H), 0.86-1.18 (m, 2 H), 0.50-0.62 (m, 1 H), 0.25-0.41 (m, 1 H). |
| 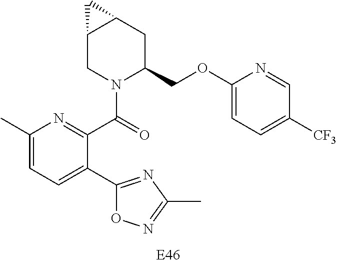 | D22 and D51 | (1R,4S,6R)-3-{[6-methyl-3-(3-methyl-1,2,4- oxadiazol-5-yl)-2-pyridinyl]carbonyl}-4-({[5- (trifluoromethyl)-2-pyridinyl]oxy}methyl)-3- azabicyclo[4.1.0]heptane UPLC (Basic_GEN_QC): rt1 = 0.95 minutes and rt2 = 0.98 minutes (rotamers present), peak observed: 473 (M + 1). C₂₃H₂₂F₃N₅O₃ requires 474. ¹H NMR (500 MHz, CDCl₃-d) ppm 8.45 (s, 1 H), 8.29 (d, 1 H), 7.78- 7.84 (m, 1 H), 7.32 (d, 1 H), 7.00 (d, 1 H), 4.96-5.04 (m, 1 H), 4.74-4.81 (m, 1 H), 4.60-4.71 (m, 1 H), 3.60-3.68 (m, 1 H), 3.25-3.33 (m, 1 H), 2.65 (s, 3 H), 2.46 (s, 3 H), 2.27-2.34 (m, 1 H), 1.90-2.00 (m, 1 H), 1.02-1.12 (m, 1 H), 0.92-0.99 (m, 1 H), 0.66- 0.74 (m, 1 H), 0.49-0.60 (m, 1 H). |
| 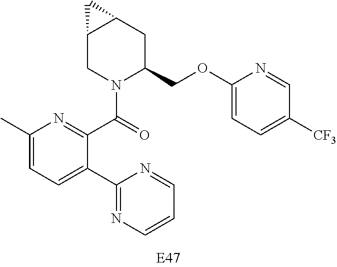 | D22 and D29 | (1R,4S,6R)-3-{[6-methyl-3-(2-pyrimidinyl)-2- pyridinyl]carbonyl}-4-({[5-(trifluoromethyl)-2- pyridinyl]oxy}methyl)-3-azabicyclo[4.1.0]heptane UPLC (Basic GEN_QC): rt1 = 0.92 minutes and rt2 = 0.96 minutes (rotamers present), peak observed: 469 (M + 1). C₂₄H₂₂F₃N₅O₂ requires 468. ¹H NMR (500 MHz, DMSO-d₆) δ ppm 8.88 (d, 2 H), 8.63-8.68 (m, 1 H), 8.47 (d, 1 H), 8.11 (dd, 1 H), 7.35-7.51 (m, 2 H), 7.08 (d, 1 H), 4.45-4.72 (m, 3 H), 3.53-3.59 (m, 1 H), 3.22-3.28 (m, 1 H), 2.54 (s, 3 H), 2.19-2.28 (m, 1 H), 1.77-1.85 (m, 1 H), 0.91-1.09 (m, 2 H), 0.57-0.67 (m, 1 H), 0.46-0.53 (m, 1 H). |
| 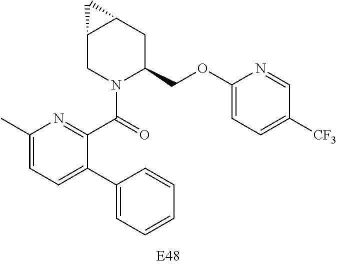 | D22 and D42 | (1R,4S,6R)-3-[(6-methyl-3-phenyl-2- pyridinyl)carbonyl]-4-({[5-(trifluoromethyl)-2- pyridinyl]oxy}methyl)-3-azabicyclo[4.1.0]heptane UPLC (Basic_GEN_QC): rt1 = 1.02 minutes and rt2 = 1.05 minutes (rotamers present), peak observed: 468 (M + 1). C₂₆H₂₄F₃N₃O₂ requires 467. ¹H NMR (400 MHz, DMSO-d₆) δ ppm 7.97-8.07 (m, 1 H), 7.65- 7.75 (m, 1 H), 7.32 (d, 1 H), 6.90-7.13 (m, 5 H), 6.88 (d, 1 H), 6.55 (d, 1 H), 3.78-4.04 (m, 3 H), 3.02-3.20 (m, 1 H), 2.81 (dd, 1 H), 1.87 (s, 3 H), 1.07-1.37 (m, 2 H), 0.35-0.66 (m, 2 H), −0.11-0.17 (m, 1 H), −1.22-- 0.90 (m, 1 H). |
| 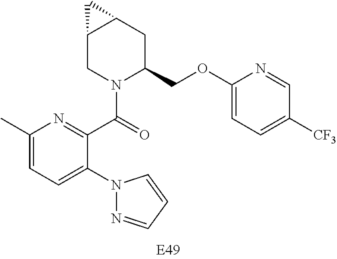 | D22 and D37 | (1R,4S,6R)-3-{[6-methyl-3-(1H-pyrazol-1-yl)-2- pyridinyl]carbonyl}-4-({[5-(trifluoromethyl)-2- pyridinyl]oxy}methyl)-3-azabicyclo[4.1.0]heptane UPLC (Basic QC_50_800_POS): rt1 = 0.92 minutes and rt2 = 0.94 minutes (rotamers present), peak observed: 458 (M + 1). C₂₃H₂₂F₃N₅O₂ requires 457. ¹H NMR (500 MHz, DMSO-d₆) δ ppm −0.04 (br. s., 1 H) 0.59-0.71 (m, 1 H) 0.88-0.95 (m, 1 H) 0.96-1.02 (m, 1 H) 1.64-1.86 (m, 1 H) 2.07-2.17 (m, 1 H) 2.27 (br. s., 3 H) 3.26-3.42 (m, 1 H) 3.56-3.71 (m, 1 H) 4.30-4.59 (m, 3 H) 6.53-6.58 (m, 1 H) 7.04 (d, 1 H) 7.46 (d, 1 H) 7.74 (s, 1 H) 8.01 (d, 1 H) 8.04-8.15 (m, 2 H) 8.41 (s, 1 H) |
| 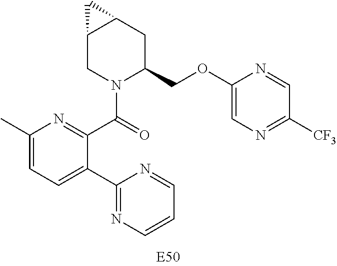 | D68 and D59 | (1R,4S,6R)-3-{[6-methyl-3-(2-pyrimidinyl)-2- pyridinyl]carbonyl}-4-({[5-(trifluoromethyl)-2- pyrazinyl]oxy}methyl)-3-azabicyclo[4.1.0]heptane UPLC (Basic GEN_QC): rt1 = 0.87 minutes and rt2 = 0.91 minutes (rotamers present) peak observed: 471 (M + 1) C₂₃H₂₁F₃N₆O₂ requires 470. ¹H NMR (500 MHz, DMSO-d₆) δ ppm 0.47-0.52 (m, 1 H) 0.58-0.67 (m, 1 H) 0.93-1.09 (m, 2 H) 1.79- 1.92 (m, 1 H) 2.21-2.29 (m, 1 H) 2.52 (s, 3 H) 3.21- 3.28 (m, 1 H) 3.55-3.60 (m, 1 H) 4.55-4.78 (m, 3 H) 7.44 (d, 1 H) 7.48 (t, 1 H) 8.46 (d, 1 H) 8.50-8.52 (m, 1 H) 8.80-8.82 (m, 1 H) 8.87 (d, 2 H) |
| 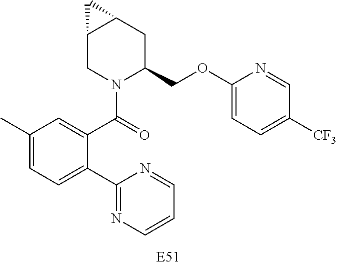 | D22 and 5-methyl-2- (2-pyrimidinyl)benzoic acid | (1R,4S,6R)-3-{[5-methyl-2-(2- pyrimidinyl)phenyl]carbonyl}-4-({[5- (trifluoromethyl)-2-pyridinyl]oxy}methyl)-3- azabicyclo[4.1.0]heptane UPLC (Basic GEN_QC): rt1 = 0.99 minutes and rt2 = 1.04 minutes (rotamers present) peak observed: 469 (M + 1) C₂₅H₂₃F₃N₄O₂ requires 468. ¹H NMR (500 MHz, CHLOROFORM-d) δ ppm 0.27- 0.35 (m, 1 H) 0.71-0.81 (m, 1 H) 0.80-1.17 (m, 2 H) 1.92-2.02 (m, 1 H) 2.25-2.34 (m, 1 H) 2.44 (s, 3 H) 3.46 (d, 1 H) 4.54-4.79 (m, 2 H) 4.76 (d, 1 H) 4.88- 4.98 (m, 1 H) 6.87 (d, 1 H) 7.09-7.16 (m, 1 H) 7.10- 7.37 (m, 2 H) 7.76-7.86 (m, 1 H) 8.26 (d, 1 H) 8.47- 8.54 (m, 1 H) 8.68-8.88 (m, 2 H) |

### Example 52

### (1R,4S,6R)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane (E52)

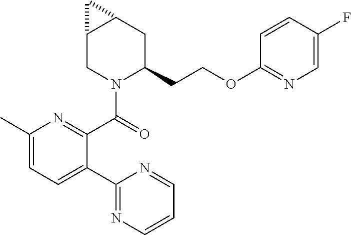

To a suspension of 6-methyl-3-(2-pyrimidinyl)-2-pyridinecarboxylic acid D59 (0.701 g) in DCM (3.2 ml) at room temperature, a solution of pentafluorophenol (0.390 g, 2.118 mmol) in DCM (1.6 ml) was added, followed by a solution of N,N′-dicyclohexylcarbodiimide (0.437 g, 2.118 mmol) in DCM (1.6 ml), added dropwise in about 15 minutes. The resulting pink-orange mixture was stirred at room temperature for 1 hour and 30 minutes: the suspension became red-pink.

Then a solution of (1R,4S,6R)-4-{2-[(5-fluoro-2-pyridinyl)oxy]ethyl}-3-azabicyclo[4.1.0]heptane D73 (0.455 g) in DCM (2 ml), was added, followed by TEA (0.537 ml, 3.85 mmol) dropwise. The mixture was stirred at room temperature for 3 hours and 30 minutes, then it was filtered and the precipitate was washed with DCM. The filtrate was washed with HCl 1M solution (2×15 ml), NaOH 1N solution (2×15 ml) and water (15 ml), dried over a phase separator and concentrated to obtain 0.81 g of a brown solid.

This crude was purified by flash chromatography (Biotage SP1, SNAP 55 g NH cartridge, eluting mixture Cy/EtOAc 5:5 in 7 CV, 3:7 in 2 CV, 3:7 in 5 CV) to obtain 0.65 g of a white foam.

It was purified by flash chromatography (SP1, SNAP 25 g Si cartridge, eluting mixture Cy/EtOAc 1:9 in 7.5 CV, 0:10 in 1 CV, 0:10 in 13 CV) to obtain a white foam (0.522 g).

To eliminate residual EtOAc, the product was dissolved in MeOH and concentrated under vacuum to obtain the title compound E52 (0.507 g) as white foam.

UPLC (IPQC): rt1=1.05 minutes and rt2=1.13 minutes (rotamers present) peak observed: 434 (M+1) C₂₄H₂₄FN₅O₂ requires 433.

¹H NMR (400 MHz, CHLOROFORM-d) δ ppm 0.59-0.70 (m, 1H) 0.74-1.17 (m, 3H) 1.65-1.87 (m, 1H) 1.87-2.00 (m, 1H) 2.06-2.22 (m, 1H) 2.36-2.52 (m, 1H) 2.62 (s, 3 H) 3.40-3.48 (m, 1H) 3.63 (d, 1H) 4.38-4.59 (m, 2H) 4.80-4.89 (m, 1H) 6.70-6.82 (m, 1H) 7.07-7.40 (m, 3H) 7.87 (d, 1H) 8.48-8.55 (m, 1H) 8.81 (d, 2H)

### Example 53

### (1R,4S,6R)-3-{[6-methyl-3-(2-pyrimidinyl)-2-pyridinyl]carbonyl}-4-(2-{[5-(trifluoromethyl)-2-pyridinyl]oxy}ethyl)-3-azabicyclo[4.1.0]heptane (E53)

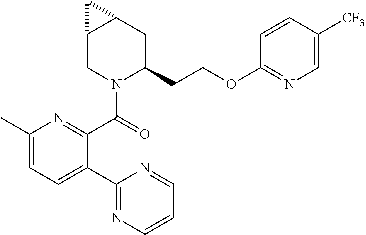

To a solution of 6-methyl-3-(2-pyrimidinyl)-2-pyridinecarboxylic acid D59 (80 mg) and DCC (49.9 mg, 0.242 mmol), under nitrogen was added pentafluorophenol (44.6 mg, 0.242 mmol) and the reaction mixture was shaken for 2 hours at room temperature. Then (1R,4S,6R)-4-(2-{[5-(trifluoromethyl)-2-pyridinyl]oxy}ethyl)-3-azabicyclo[4.1.0]heptane D88 (63 mg) in DCM (2 ml) followed by TEA were added and the reaction mixture was shaken for 24 hours and left without shaking for another 24 hours. Then the white solid was filtered and washed with DCM, the organic filtrate was washed with HCl 1M and NaOH 1M and then water. The organic solvent was removed to give a crude, which was purified by preparative HPLC (method: BASIC 1) to give the title compound E53 (50 mg).

UPLC (IPQC): rt1=1.18 minutes and rt2=1.24 minutes (rotamers present) peak observed: 484 (M+1) C₂₅H₂₄F₃N₅O₂ 483.

¹H NMR (500 MHz, DMSO-d₆) δ ppm 0.51-0.57 (m, 1H) 0.57-0.63 (m, 1H) 0.85-0.94 (m, 1H) 0.97-1.05 (m, 1H) 1.72-2.11 (m, 3H) 2.32-2.42 (m, 1H) 2.53 (s, 3H) 3.29-3.35 (m, 1H) 3.60 (dd, 1H) 4.44 (m, 2H) 4.56-4.63 (m, 1H) 7.02 (d, 1H) 7.43 (d, 1H) 7.47 (t, 1H) 8.05 (d, 1H) 8.43 (d, 1H) 8.60-8.64 (m, 1H) 8.88 (d, 2H)

### Example 54

### (1R,4S,6R)-3-[(3-chloro-6-methyl-2-pyridinyl)carbonyl]-4-{2-[(5-fluoro-2-pyridinyl)oxy]ethyl}-3-azabicyclo[4.1.0]heptane (E54)

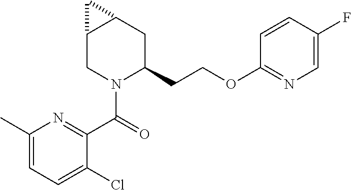

Into a 10 ml round bottomed flask 3-chloro-6-methyl-2-pyridinecarboxylic acid lithium salt D81 (0.033 g) was added and dissolved in DCM (3 ml). To this solution DIPEA (0.177 ml, 1.016 mmol) and TBTU (0.060 g, 0.186 mmol) were added and the resulting mixture was stirred at room temperature for 30 minutes. Then a DCM solution (2 ml) of (1R,4S,6R)-4-{2-[(5-fluoro-2-pyridinyl)oxy]ethyl}-3-azabicyclo[4.1.0]heptane D73 (0.040 g) was added and the resulting mixture left under stirring at room temperature for 6 hours. Volatiles were removed to give a crude white solid. This material was purified by column chromatography on silica gel (flash master, 50 g Si cartridge, eluting with DCM/MeOH from 100:0 to 90:10). Collected fractions gave the title compound EM (0.051 g) as a slightly yellow oil.

UPLC (IPQC): rt=1.11 minutes, peak observed: 390 (M+1) C₂₀H₂₁ClFN₃O₂ requires 389.

¹H NMR (500 MHz, DMSO-d₆) δ ppm 0.06 (d, 1H) 0.69-0.80 (m, 1H) 0.94-1.06 (m, 1H) 1.06-1.14 (m, 1H) 1.67-1.89 (m, 3H) 2.23 (dd, 1H) 2.34 (s, 3H) 3.26 (m, 1H) 3.34 (dd, 1H) 4.06-4.21 (m, 2H) 4.47 (d, 1H) 6.58 (dd, 1H) 7.29 (d, 1H) 7.60-7.72 (m, 1H) 7.79 (d, 1H) 8.09 (d, 1H)

### Example 55

### (1R,4S,6R)-4-{2-[(4,6-dimethyl-2-pyrimidinyl)oxy]ethyl}-3-{[6-methyl-3-(2-pyrimidinyl)-2-pyridinyl]carbonyl}-3-azabicyclo[4.1.0]heptane (E55)

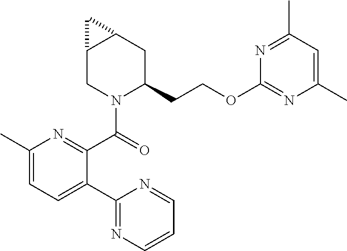

To a solution of 2-((1R,4S,6R)-3-{[6-methyl-3-(2-pyrimidinyl)-2-pyridinyl]carbonyl}-3-azabicyclo[4.1.0]hept-4-yl)ethanol D94 (52 mg) in dry THF (5 ml) under nitrogen, sodium hydride (8.76 mg, 0.219 mmol) was added portionwise and the resulting solution was stirred at room temperature for 10 minutes.

2-chloro-4,6-dimethylpyrimidine (31.2 mg, 0.219 mmol) was then added and the reaction mixture was stirred at room temperature for 12 hours. Water (20 ml) and EtOAc (30 ml) were added, the organic layer was separated, dried over sodium sulphate, filtered and concentrated under vacuum, to give a crude that was purified on SP1 (SNAP 10 g silica column, EtOAc to EtOAc/MeOH 8:2 as eluent). It was obtained 25 mg of a mixture which was purified by preparative HPLC (method BASIC 2), to give the title compound E55 (9.6 mg) as a white solid.

UPLC (IPQC): rt1=0.86 minutes and rt2=0.97 minutes (rotamers present) peak observed: 445 (M+1) C₂₅H₂₈N₆O₂ requires 444.

### Example 56

### (1R,4S,6R)-3-{[6-methyl-3-(2-pyrimidinyl)-2-pyridinyl]carbonyl}-4-[2-(3-pyridinyloxy)ethyl]-3-azabicyclo[4.1.0]heptane (E56)

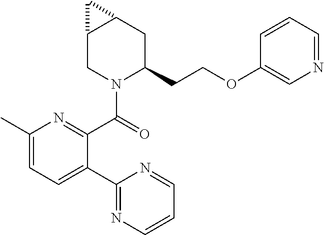

To a solution of 6-methyl-3-(2-pyrimidinyl)-2-pyridinecarboxylic acid D59 (0.029 g) in DCM (1 ml) was added a solution of pentafluorophenol (0.023 g, 0.124 mmol) in DCM (1 ml) and dropwise a solution of DCC (0.026 g, 0.124 mmol) in DCM (1 ml); the resulting reaction mixture was left under stirring for 2.5 hours. After this time a solution of (1R,4S,6R)-4-[2-(3-pyridinyloxy)ethyl]-3-azabicyclo[4.1.0]heptane D90 (0.027 g) in DCM (2 ml) was added, followed by TEA (0.017 ml, 0.124 mmol). The reaction mixture was left under stirring at room temperature for 16 hours. Volatiles were removed and the residue was purified by column chromatography on silica gel (flash master, 50 g Si cartridge, eluting with DCM/MeOH from 100:0 to 80:20). Collected fractions gave the title compound E56 (41 mg) as an off-white solid.

UPLC (IPQC): rt1=0.63 minutes and rt2=0.70 minutes (rotamers present) peak observed: 416 (M+1) C₂₄H₂₅N₅O₂ requires 415.

¹H NMR (500 MHz, DMSO-d₆) δ ppm 0.20-0.27 (m, 1H) 0.72-0.79 (m, 1H) 0.96-1.12 (m, 2H) 1.68-1.81 (m, 2H) 1.87-1.95 (m, 1H) 2.24-2.37 (m, 1H) 2.42 (s, 3H) 3.26-3.37 (m, 1H) 3.50-3.57 (m, 1H) 3.94-4.02 (m, 1H) 4.04-4.12 (m, 1H) 4.45 (d, 1H) 7.07-7.16 (m, 1H) 7.18-7.25 (m, 1H) 7.31-7.47 (m, 2H) 7.91-8.01 (m, 1H) 8.03-8.09 (m, 1H) 8.39-8.44 (m, 1H) 8.83 (d, 2H)

### Experimental for (1S,4S,6S) [4.1.0] CIS compounds (referring to scheme 4 and 5)

### Description 97: (2S)-2-amino-4-penten-1-ol (D97)

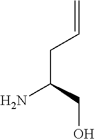

In a 20 L reactor, to a suspension of (25)-2-amino-4-pentenoic acid (available from Sigma-Aldrich #285013) (200 g, 1319 mmol) in THF dry (3200 ml) stirred under nitrogen at 0° C. was added a solution of LiAlH₄ (1600 ml, 1600 mmol) 1 M in THF dropwise in 1.5 hours (maintaining internal temperature between 0° C. and 5° C.). The reaction mixture was stirred at 25° C. for 2 hours (white suspension). The check by TLC (DCM/MeOH 1:1, AcOH 0.5% ninihydrine) showed reaction to be completed. The reaction mixture was cooled to 0° C. and was quenched by adding in sequence: 60.7 ml of water (1 ml H₂O×1 g of LiAlH₄)+60.7 ml of NaOH 1 N (1 ml NaOH 1M×1 g of LiAlH₄)+182 ml of water (3 ml H₂O×1 g of LiAlH₄). The suspension was stirred at room temperature for 1 hour then the precipitate was filtered over sodium sulphate (gooch n3) and washed with Et₂O (6 L) and DCM (4 L). The solvent was evaporated (temperature bath 30° C.) to obtain the crude title compound D97 (110 g) as pale-orange oil. MS: (ES/+) m/z: 102 (M+1) C₅H₁₁NO requires 101. ¹H NMR (400 MHz, DMSO-d₆) δ ppm 1.39 (br. s, 2H) 1.81-1.96 (m, 1H) 2.06-2.19 (m, 1H) 2.59-2.73 (m, 1H) 3.14 (dd, 1H) 3.26 (dd, 1H) 4.48 (br. s, 1H) 4.91-5.09 (m, 2H) 5.71-5.92 (m, 1H)

### Description 98: 1,1-dimethylethyl (2-{[(1S)-1-(hydroxymethyl)-3-buten-1-yl]amino}-2-oxoethyl)carbamate (non-preferred name) (D98)

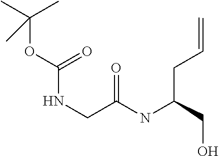

In a 5 L reactor, to a solution of (2S)-2-amino-4-penten-1-ol D97 (110 g of the crude title compound prepared in the description D97) in THF (660 ml) and MeOH (440 ml) stirred at 0° C. (+5° C. internal) was added triethylamine (182 ml, 1305 mmol) and 2,5-dioxo-1-pyrrolidinyl N-{[(1,1-dimethylethyl)oxy]carbonyl}glycinate (available from Sigma-Aldrich #15423) (237 g, 870 mmol) portionwise over 15 minutes. The reaction mixture was stirred at 2° C. (internal temperature) for 3 hours. TLC check (TLC-NH₂, DCM/MeOH 95:5, potassium permanganate) showed residual starting material. Further 2,5-dioxo-1-pyrrolidinyl N-{[(1,1-dimethylethyl)oxy]carbonyl}glycinate (60 g, 220 mmol) was added and the mixture stirred at 2° C. for 1 hour. TLC check (TLC-NH₂, DCM/MeOH 95:5, potassium permanganate) showed residual starting material. Further 2,5-dioxo-1-pyrrolidinyl N-{[(1,1-dimethylethyl)oxy]carbonyl}glycinate (40 g, 146 mmol) was added and the mixture stirred at 2° C. for 1 hours. TLC check showed residual starting material but the work-up was carried out. The reaction mixture was poured into aqueous saturated solution of NH₄Cl (3400 ml) and EtOAc (1375 ml), then the phases were separated and the aqueous layer was back-extracted with EtOAc (1375 ml). The combined organic layers were washed with NaHCO₃ aqueous saturated solution (1031 ml) dried (Na₂SO₄) and evaporated to give crude material (268 g, deep brown). This residue was triturated with Et₂O (687 ml) for 1 hour at 25° C. The solid was filtered (gooch n3), washed with Et₂O (200 ml) and dried under vacuum to give the title compound D98 (87 g) as pale brown solid. Mother liquors (deep brown) were evaporated and the residue chromatographed (Biotage 75 L, silica column, eluting with DCM/MeOH 98:2, 95:5) to give 34 g of residual brown product that was triturated with Et₂O (200 ml). The solid was filtered, washed with Et₂O and dried under vacuum to give a further batch of the title compound D98 (26 g) as pale brown solid.

MS: (ES/+) m/z: 259 (M+1). C₁₂H₂₂N₂O₄ requires 258. ¹H NMR (400 MHz, CDCl₃) δ ppm 1.47 (s, 9H) 2.22-2.43 (m, 2H) 2.68-2.83 (m, 1H) 3.50-3.86 (m, 4H) 3.94-4.09 (m, 1H) 5.00-5.25 (m, 3H) 5.64-5.89 (m, 1H) 6.17-6.44 (m, 1H)

### Description 99: 1,1-dimethylethyl {2-[(4S)-2,2-dimethyl-4-(2-propen-1-yl)-1,3-oxazolidin-3-yl]-2-oxoethyl}carbamate (non-preferred name) (D99)

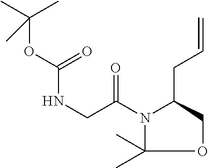

To a suspension of 1,1-dimethylethyl (2-{[(1S)-1-(hydroxymethyl)-3-buten-1-yl]amino}-2-oxoethyl)carbamate D98 (37 g) in toluene (370 ml) stirred at 25° C. were added 2,2-bis(methyloxy)propane (370 ml, 3020 mmol) and p-toluenesulfonic acid monohydrate (3.7 g, 19.45 mmol). The reaction mixture was stirred at reflux (85° C. internal, oil bath 105° C.) for 1.5 hour (clear solution). The check by TLC (DCM/MeOH 95:5) showed the reaction to be completed. The solvent was evaporated to obtain a brown oil that was chromatographed (Biotage 75 L, silica, eluting with Cy/EtOAc 8:2, 7:3) to give the title compound D99 (30 g) as yellow oil.

UPLC (Acid GEN_QC): rt=0.69 minutes, peak observed: 299 (M+1). C₁₅H₂₆N₂O₄ requires 298. ¹H NMR (400 MHz, CDCl₃) δ ppm 1.46 (s, 9H) 1.54 (s, 3H) 1.67 (s, 3H) 2.30-2.49 (m, 2H) 3.71-4.05 (m, 5H) 5.04-5.22 (m, 2H) 5.37-5.52 (m, 1H) 5.65-5.81 (m, 1H)

### Description 100: {2-[(4S)-2,2-dimethyl-4-(2-propen-1-yl)-1,3-oxazolidin-3-yl]-2-oxoethyl}amine trifluoromethansulfonate (1:1) (D100)

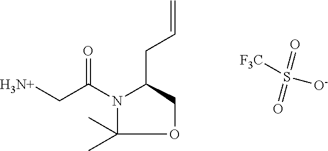

To a solution of 1,1-dimethylethyl {2-[(4S)-2,2-dimethyl-4-(2-propen-1-yl)-1,3-oxazolidin-3-yl]-2-oxoethyl}carbamate D99 (28.67 g) in DCM (300 ml) 2,6-dimethylpyridine (27.9 ml, 240 mmol) was added followed by trimethylsilyl trifluoromethanesulfonate (34.7 ml, 192 mmol); the mixture was stirred at room temperature for 30 minutes. The reaction was quenched with 2 ml of water and the solvent was removed under reduced pressure, the residue was charged on a Biotage 75 L column (eluting with DCM/MeOH 100:0 then 98:2 then 96:4 then 85:15). Evaporation of the solvent gave the title compound D100 (21 g).

UPLC (Acid FINAL_QC): rt=0.36 minutes, peak observed: 199 (M+1-CHF₃O₃S) C₁₀H₁₈N₂O₂.CHF₃O₃S requires 348. ¹H NMR (400 MHz, DMSO-d₆) δ ppm 1.40 (s, 3H), 1.50 (s, 3H), 2.17-2.43 1.40 (m, 2H), 3.68-3.98 (m, 4H), 3.99-4.09 (m, 1H), 4.83-5.40 (m, 2H), 5.58-5.97 (m, 1H), 7.63-8.36 (br.s., 2H)

### Description 101: (48)-3-(diazoacetyl)-2,2-dimethyl-4-(2-propen-1-yl)-1,3-oxazolidine (D101)

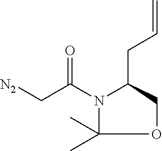

{2-[(4S)-2,2-dimethyl-4-(2-propen-1-yl)-1,3-oxazolidin-3-yl]-2-oxo ethyl}amine trifluoromethansulfonate D100 (67.0 g) was dissolved in DCM (670 ml) and pH=5 Buffer solution (670 ml) and cooled to 2° C. (internal). Sodium nitrite (26.5 g, 385 mmol) dissolved in water (134 ml) was added dropwise to the reaction mixture stirred at 2° C. over 30 minutes. The reaction mixture was stirred at 3° C. for 2.5 hours. Phases were separated. Water phase was back-extracted with DCM (1×670 ml, 1×335 ml). The combined organic layers, dried (Na₂SO₄), were evaporated (bath temperature 30° C.) to give 43 g of crude product. This crude was purified over silica pad [(230-400 Mesh) eluting with Cy/EtOAc 8:2, 7:3, 6:4] to give the title compound D101 (36.58 g) as pale yellow oil.

UPLC (Acid GEN_QC): rt=0.59 minutes, peak observed: 210 (M+1) C₁₀H₁₅N₃O₂ requires 209. ¹H NMR (400 MHz, CDCl₃) δ ppm 1.58 (s, 3H) 1.69 (s, 3H) 2.25-2.50 (m, 2H) 3.43-3.70 (m, 1H) 3.82-4.01 (m, 2H) 4.84 (s, 1H) 5.09-5.24 (m, 2H) 5.63-5.84 (m, 1H)

### Description 102: (5aS,6aS,7aS)-3,3-dimethylhexahydro-5H-cyclopropa[d][1,3]oxazolo[3,4-a]pyridin-5-one and (5aR,6aR,7aS)-3,3-dimethylhexahydro-5H-cyclopropa[d][1,3]oxazolo[3,4-a]pyridin-5-one (D102A syn/D102B anti)

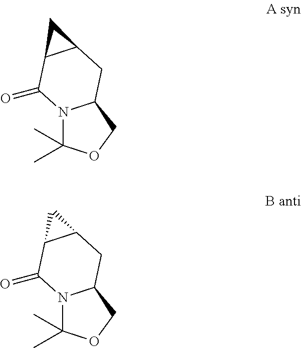

(4S)-3-(diazoacetyl)-2,2-dimethyl-4-(2-propen-1-yl)-1,3-oxazolidine D101 (36.5 g) dissolved in DCM (365 ml) was added dropwise at 25° C. to a suspension of rhodium(II) acetate dimer (3.85 g, 8.72 mmol) in DCM (183 ml) over 2.5 hours. The resulting mixture was stirred at 25° C. for 30 minutes. From TLC (Cy/EtOAc 1:1): no more starting material. The mixture was filtered (gooch n 3), concentrated and chromatographed twice (over silica 230-400 Mesh, eluting with Cy/EtOAc 7:3, 6:4) to give three fractions that after trituration with n-heptane (40 ml, for each fraction) gave the following three batches:

D102B/D102A 95:3 (10.3 g, anti as major isomer anti/syn 95:3) HPLC (walk up): rt1=3.09 minutes and rt2=3.14 minutes;

D102A/D102B 31:68 (4.47 g, anti/syn roughly 31:68) HPLC (walk up): rt1=3.05 minutes and rt2=3.11 minutes;

D102A/D102B (10.5 g, D 102A syn as major isomer). HPLC (walk up): rt1=3.08 minutes and rt2=3.16 minutes.

¹H NMR (500 MHz, CDCl₃) δ ppm 3.87-4.02 (m, 2H), 3.32 (t, 1H), 2.29-2.38 (m, 1H), 1.57 (s, 3H), 1.45-1.51 (m, 1H), 1.43 (s, 3H), 1.36-1.42 (m, 1H), 1.12-1.20 (m, 1H), 1.06-1.12 (m, 0H), 0.45-0.54 (m, 1H) 662 mg of this third batch of D102A/D102B were taken and purified by flash chromatography (Snap-50 g silica gel column, EtOAc/Cy from 100% Cy to 30:70). From this purification it was obtained a batch of almost pure cis isomer (the title compound D102A) (298 mg) as white solid, and a 347 g batch of a mixture of cis/trans isomers (75:25) as a colourless oil.

UPLC (Basic GEN_QC): rt=0.48 minutes, peak observed: 182 (M+1). C₁₀H₁₅NO₂ requires 181. ¹H NMR (400 MHz, CDCl₃) δ ppm 3.98-4.10 (m, 2H) 3.36-3.45 (m, 1H) 2.37-2.47 (m, 1H) 1.66 (s, 3H) 1.53-1.61 (m, 1H) 1.52 (s, 3H) 1.42-1.50 (m, 1H) 1.14-1.29 (m, 2H) 0.59 (m, 1H)

### Description 103: (1S,4S,6S)-4-(hydroxymethyl)-3-azabicyclo[4.1.0]heptan-2-one (D103)

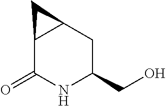

(5 aS,6 aS,7 aS)-3,3-dimethylhexahydro-5H-cyclopropa [d][1,3]oxazolo[3,4-a]pyridin-5-one D102A (3.56 g) was dissolved in HCl 6 M in water (25 ml, 150 mmol) into a 250 ml-round bottomed flask and the mixture was stirred at 40° C.: after 4 hours the reaction was complete. The solvent was evaporated at reduced pressure using a rotavapor (bath temperature: 40° C.). The oily residue was stripped with toluene and the residue dried under high vacuum for 3 hours, obtaining the title compound D103 as white solid (2.843 g).

UPLC (IPQC): rt=0.31 minutes, peak observed: 142 (M+1) C₇H₁₁NO₂ requires 141. ¹H NMR (500 MHz, DMSO-d₆) d ppm 0.56-0.68 (m, 1H) 0.93-1.05 (m, 1H) 1.30-1.39 (m, 1H) 1.39-1.48 (m, 1H) 1.57-1.67 (m, 1H) 1.98-2.09 (m, 1H) 3.17-3.29 (m, 2H) 3.31-3.40 (m, 1H) 6.89-7.13 (m, 1H)

### Description 104: 1,1-dimethylethyl (1S,4S,6S)-4-(hydroxymethyl)-3-azabicyclo[4.1.0]heptane-3-carboxylate (D104)

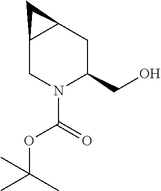

(1S,4S,6S)-4-(hydroxymethyl)-3-azabicyclo[4.1.0]heptan-2-one D103 (3.839 g) was suspended in THF (40 ml) then BH₃.THF 1 M THF solution (136 ml, 136 mmol) was added slowly (over 5 minutes) and the resulting mixture stirred at reflux for 2 hours. The mixture was cooled to room temperature and then to 0° C. using an ice/water bath. MeOH (25 ml) was slowly added and, when the gas evolution stopped, HCl 3 M (140 ml, 420 mmol) was slowly added and the resulting mixture was stirred again at 85° C. for 1 hour. The mixture was cooled again to room temperature.

A second reaction mixture was prepared: (1S,4S,6S)-4-(hydroxymethyl)-3-azabicyclo[4.1.0]heptan-2-one D103 (100 mg) was suspended in THF (0.5 ml), then BH₃.THF (3.6 ml, 3.60 mmol) was added slowly (over 1 minute) and the resulting mixture stirred at reflux for 2 hours. This second mixture was chilled to room temperature, then HCl (3.6 ml, 10.80 mmol) was slowly added and the resulting mixture was stirred again at 75° C. for 1 hour. This mixture was chilled again to room temperature and then it was added to the first mixture to form a single mixture.

NaOH 3 M (140 ml, 420 mmol) was slowly added to the acidic mixture described above, then additional NaOH (50 ml, 150 mmol) was added in order to get a pH value of about 10. Boc₂O (7.13 ml, 30.7 mmol) was added dissolved in THF (30 ml) and the resulting biphasic mixture was stirred vigorously at room temperature overnight. New Boc₂O (4.57 ml, 19.70 mmol) was added dissolved in THF (20 ml) and the mixture stirred vigorously at room temperature for 1.5 hours. EtOAc (100 ml) was added to the mixture and the phases were separated. The water phase was extracted with EtOAc (3×100 ml) and all the organic fractions were mixed together. The so obtained organic solution was washed with brine (3×150 ml), dried over Na₂SO₄ and evaporated at reduced pressure, obtaining the crude target material as pale yellow oil (14 g). This material was purified by Biotage (Snap-340 g silica gel column, from pure Cy to EtOAc/Cy 70:30). It was obtained the title compound D104 (5.695 g) as colourless oil. MS: (ES/+) m/z: 228 (M+1) 128 (M+1-Boc). C₁₂H₂₁NO₃ requires 227. ¹H NMR (500 MHz, DMSO-d₆) δ ppm 4.43 (t, 1H), 4.00-4.20 (m, 1H), 3.34-3.45 (m, 1H), 3.25-3.31 (m, 2H), 2.10-2.23 (m, 1H), 2.00-2.10 (m, 1H), 1.30 (s, 9H), 0.86-0.99 (m, 2H), 0.66-0.77 (m, 1H), 0.51-0.61 (m, 1H), −0.04-0.05 (m, 1H)

### Description 105: 3-(2-pyrimidinyl)-2-pyridinecarboxylic acid (D105)

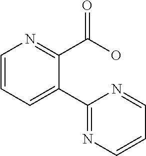

2-(tributylstannanyl)pyrimidine (445 mg, 1.206 mmol) was dissolved in 1,4-Dioxane (2 ml). To the stirred solution 3-bromo-2-pyridinecarbonitrile (200 mg, 1.093 mmol) was added dissolved in 1,4-Dioxane (2 ml) and followed by Pd(PPh₃)₄ (125 mg, 0.108 mmol).

The mixture was heated by microwave irradiation at 160° C. for 60 minutes: an UPLC check showed an almost complete conversion.

The solvent was removed at reduced pressure and the dark brown residue partitioned between water (30 ml) and Et₂O (30 ml).

The aqueous phase was extracted with Et₂O (3×20 ml); all the organic fractions were joined together, dried over Na₂SO₄, filtered and evaporated at reduced pressure, obtaining the crude target material as grey solid (719 mg).

This material was purified by Biotage (Snap-50 G silica gel column, from pure cyclohexane to EtOAc/cyclohexane 50:50).

After evaporation at reduced pressure of the pure collected fractions it was obtained the desired cyano derivative as white solid (114.7 mg).

This material was dissolved in Ethanol (2 ml) into an 8 ml-capped vial and a solution of NaOH (79 mg, 1.975 mmol) in Water (1 ml) was added in one portion.

The resulting mixture was stirred 5 hours at 100° C. using a PLS apparatus: 14%-UV of primary amide was still present, so new NaOH (11 mg, 0.275 mmol) was added.

The resulting mixture was stirred for other 2 hours at 100° C. using a PLS apparatus: almost complete conversion.

The solvent was evaporated at reduced pressure, obtaining the desired acid as sodium salt, but containing an excess of NaOH.

This material was taken up in water (0.5 ml) and adjusted to pH=2 with aqueous 1M HCl solution.

The so obtained solution was loaded onto a pre-conditioned C18 column (25 g). The column was eluted with water and then ACN. The first three ACN fractions showed to contain the desired acid, so were evaporated under reduced pressure to give the title compound D105 (116 mg) as white solid.

UPLC (Basic GEN_QC): rt=0.17 minutes peak observed: 202 (M+1) C₁₀H₇N₃O₂ requires 201.

¹H NMR (400 MHz, DMSO-d₆) δ ppm 8.47 (dd, 1H) 8.71 (dd, 1H) 8.94 (d, 2H) 13.16 (br. s., 1H)

### Description 106: 1,1-dimethylethyl (1S,4S,6S)-4-({[4-(trifluoromethyl)-2-pyridinyl]oxy}methyl)-3-azabicyclo[4.1.0]heptane-3-carboxylate (D106)

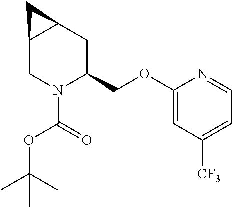

1,1-dimethylethyl (1S,4S,6S)-4-(hydroxymethyl)-3-azabicyclo[4.1.0]heptane-3-carboxylate D104 (250 mg) and 2-fluoro-4-(trifluoromethyl)pyridine (200 mg, 1.210 mmol) were dissolved in DMF (5 ml) and the stirred mixture was chilled at 0° C.

NaH (57 mg, 1.425 mmol) was then added in one portion (moderate gas evolution).

The mixture was stirred at 0° C. for 15 minutes, then at room temperature for 60 minutes: almost complete conversion.

The reaction was quenched by a slow and careful addition of NaHCO₃ saturated solution (10 ml) (gas evolution), then the solvents were evaporated at reduced pressure.

The residue was partitioned between water (50 ml) and Et₂O (50 ml); water layer extracted with Et₂O (3×20 ml).

The organic phases were joined, dried over Na₂SO₄ and evaporated at reduced pressure.

The so obtained yellow oily residue (300 mg) was purified by Biotage (Snap-100 G silica gel column, EtOAc/Cy from only Cy to 20:80).

After evaporation at reduced pressure of the pure collected fractions it was obtained the title compound D106 as colorless oil (250 mg).

UPLC (IPQC): rt=1.41 minutes, peak observed: 373 (M+1) C₁₈H₂₃F₃N₂O₃ requires 372

### Description 107: (1S,4S,6S)-4-({[4-(trifluoromethyl)-2-pyridinyl]oxy}methyl)-3-azabicyclo[4.1.0]heptane (D107)

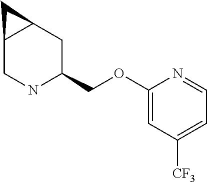

To 1,1-dimethylethyl (1S,4S,6S)-4-({[4-(trifluoromethyl)-2-pyridinyl]oxy}methyl)-3-azabicyclo[4.1.0]heptane-3-carboxylate D106 (245 mg) a solution of TFA (1.5 ml, 19.47 mmol) in DCM (4.5 ml) was added dropwise over 30 seconds: the mixture was stirred at room temperature monitoring the reaction by LCMS.

After 1.5 hours the deprotection was complete, so the whole mixture was loaded onto an SCX-10 G column, firstly eluted with DCM (20 ml), then MeOH (20 ml): the target material was then collected eluting with NH₃ 2N in MeOH (20 ml).

After evaporation at reduced pressure of the ammoniacal solution it was obtained the title compound D107 as colorless oil (164 mg).

UPLC (IPQC): rt=0.65 minutes, peak observed: 273 (M+1) C₁₃H₁₅F₃N₂O requires 272

¹H NMR (400 MHz, CHLOROFORM-d) δ ppm 0.37-0.45 (m, 1H) 0.60-0.69 (m, 1H) 0.85-0.97 (m, 1H) 0.98-1.09 (m, 1H) 1.34-1.47 (m, 1H) 1.95-2.07 (m, 1H) 2.80-2.91 (m, 1H) 3.19 (dd, 1H) 3.27 (d, 1H) 4.06 (dd, 1H) 4.31 (dd, 1H) 6.98-7.02 (m, 1H) 7.04-7.13 (m, 1H) 8.27 (d, 1H)

### Description 108: 1,1-dimethylethyl (1S,4S,6S)-4-formyl-3-azabicyclo[4.1.0]heptane-3-carboxylate (D108)

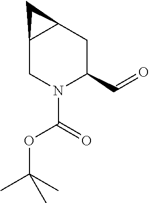

Dess-Martin periodinane (1612 mg, 3.80 mmol) was added portionwise to a stirred solution of 1,1-dimethylethyl (1S,4S,6S)-4-(hydroxymethyl)-3-azabicyclo[4.1.0]heptane-3-carboxylate D104 (720 mg) in DCM (10 ml) at room temperature. The reaction mixture was stirred for 2 hours. The reaction mixture was diluted with DCM (20 ml) and quenched with 5% sodium thiosulphate in saturated NaHCO₃ aqueous solution (25 ml). The resulting biphasic mixture was stirred vigourously for 15 minutes then filtered through a hydrophobic frit washing with more DCM (3×20 ml). The combined organic phases were evaporated under reduced pressure and the residue was purified via Biotage (5%-30% EtOAc/cyclohexane; SNAP 25 SiO₂ column) to give the title compound D108 (515 mg) as colourless oil.

UPLC (Basic GEN_QC: rt=0.82 minutes, peak observed 226 (M+1) C₁₂H₁₉NO₃ requires 225.

### Description 109: 1,1-dimethylethyl (1S,4S,6S)-4-ethenyl-3-azabicyclo[4.1.0]heptane-3-carboxylate (D109)

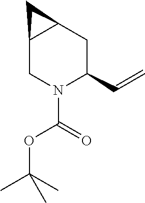

1.6M BuLi in hexane (3.38 ml, 5.4 mmol) was added dropwise to a stirred suspension of methyl(triphenyl)phosphonium bromide (1929 mg, 5.4 mmol) in THF (20 ml) at room temperature. The mixture became yellow during the addition and was almost a homogeneous solution at the end of the addition. The reaction mixture was stirred for 10 minutes then a solution of 1,1-dimethylethyl (1S,4S,6S)-4-formyl-3-azabicyclo[4.1.0]heptane-3-carboxylate D108 (510 mg) in THF (5 ml) was added. The resulting mixture was stirred overnight. The reaction was quenched with saturated NaHCO₃ aqueous solution (50 ml) and extracted with EtOAc (2×30 ml). The combined organic phases were filtered through a hydrophobic frit and evaporated under reduced pressure to give a residue which was purified via Biotage (5% EtOAc/cyclohexane; SNAP 25 SiO₂, 2 columns in series) to give the title compound D109 (322 mg) as pale yellow oil.

UPLC (Acid GEN_QC: rt=0.84 minutes, peak observed: 224 (M+1) C₁₃H₂₁NO₂ requires 223

### Description 110:1,1-dimethylethyl (1S,4S,6S)-4-(2-hydroxyethyl)-3-azabicyclo[4.1.0]heptane-3-carboxylate (D110)

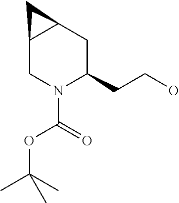

1.0 M BH₃.THF in THF (3.60 ml, 3.60 mmol) was added to a stirred solution of 1,1-dimethylethyl (1S,4S,6S)-4-ethenyl-3-azabicyclo[4.1.0]heptane-3-carboxylate D109 (268 mg) in THF (5 ml) under nitrogen at room temperature. The reaction mixture was stirred at 50° C. for 2 hours. The reaction was cooled to room temperature and quenched with water (0.1 ml).

3M NaOH solution (0.240 ml, 0.480 mmol) and 30% hydrogen peroxide solution (0.184 ml, 1.800 mmol) were added to the reaction mixture and the reaction was stirred at room temperature for 2 hours. The reaction mixture was diluted with Et₂O (50 ml) and water (50 ml), the phases were separated and the aqueous extracted with Et₂O. The combined organic phases were passed through a hydrophobic filter and evaporated under reduced pressure to give a residue which was purified via Biotage (10%-50% EtOAc/cyclohexane; SNAP 25 SiO₂ column) to give the title compound D110 (176 mg).

UPLC (Acid GEN_QC: rt=0.68 minutes, peak observed: 242 (M+1) C₁₃H₂₃NO₃ requires 241

### Description 111: 1,1-dimethylethyl (1S,4S,6S)-4-{2-[(5-fluoro-2-pyridinyl)oxy]ethyl}-3-azabicyclo[4.1.0]heptane-3-carboxylate (D111)

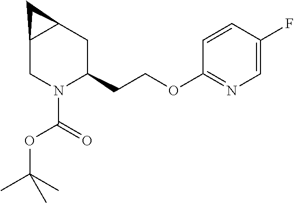

Di-tert-butyl azodicarboxylate (334 mg, 1.450 mmol) was added to stirred solution of 1,1-dimethylethyl (1S,4S,6S)-4-(2-hydroxyethyl)-3-azabicyclo[4.1.0]heptane-3-carboxylate D110 (175 mg), 5-fluoro-2-pyridinol (123 mg, 1.088 mmol) and n-tributylphosphine (0.358 ml, 1.450 mmol) in THF (5 ml) at 35° C., and the resulting mixture was stirred for 2 hours. The reaction mixture was evaporated under reduced pressure and the residue was purified via Biotage (5%-20% EtOAc/cyclohexane; 2×SNAP 25 SiO₂ columns in series) to give the title compound D111 (159 mg) as colourless oil.

UPLC (Basic GEN_QC: rt=1.07 minutes, peak observed: 337 (M+1) C₁₈H₂₅FN₂O₃ requires 336

### Description 112: (1S,4S,6S)-4-{2-[(5-fluoro-2-pyridinyl)oxy]ethyl}-3-azabicyclo[4.1.0]heptane (D112)

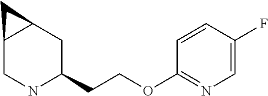

TFA (1 ml, 12.98 mmol) was added to a stirred solution of 1,1-dimethylethyl (1S,4S,6S)-4-{2-[(5-fluoro-2-pyridinyl)oxy]ethyl}-3-azabicyclo[4.1.0]heptane-3-carboxylate D111(158 mg) in DCM (3 ml) at room temperature. The mixture was stirred for 20 minutes. The reaction mixture was evaporated under reduced pressure and the residue was loaded onto a preconditioned SCX cartridge (5 g). The cartridge was eluted with MeOH and then 2M NH₃ in MeOH. The basic fractions were evaporated to give the title compound D112 (111 mg) as a pale yellow oil which solidifies to an off-white solid.

UPLC (Basic GEN_QC): rt=0.78 minutes, peak observed: 237 (M+1) C₁₃H₁₇FN₂O requires 236.

¹H NMR (400 MHz, CHLOROFORM-d) δ ppm 0.32 (q, 1H) 0.63 (td, 1H) 0.80-0.93 (m, 1H) 0.95-1.07 (m, 1H) 1.28 (ddd, 1H) 1.71-1.80 (m, 3H) 2.07 (ddd, 1H) 2.48-2.63 (m, 1H) 3.16 (dd, 1H) 3.26 (d, 1H) 4.23-4.46 (m, 2H) 6.72 (dd, 1H) 7.35 (ddd, 1H) 7.99 (d, 1H)

### Description 113: 1,1-dimethylethyl (1S,4S,6S)-4-({[4-iodo-5-(trifluoromethyl)-2-pyridinyl]oxy}methyl)-3-azabicyclo[4.1.0]heptane-3-carboxylate (D113)

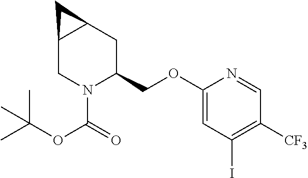

In a 40 ml screw-capped vial, 1,1-dimethylethyl (1S,4S,6S)-4-(hydroxymethyl)-3-azabicyclo[4.1.0]heptane-3-carboxylate D104 (250 mg) was dissolved in THF (16 ml) and NaH (57.2 mg, 1.430 mmol) was added. After the gas evolution was complete (about 5 minutes) 2-chloro-4-iodo-5-(trifluoromethyl)pyridine (338 mg, 1.100 mmol) was added to the reaction mixture and it was stirred for 1 hour at 67° C.

Saturated solution of NaHCO₃ (15 ml) and DCM (20 ml) were added to the reaction mixture, the aqueous layer was backextracted with DCM (2×10 ml), the biphasic system was separated through a hydrophobic filter, the organic layers were collected together, dried over Na₂SO₄, filtered and evaporated under reduced pressure.

The yellow semisolid obtained was charged on a SNAP KP-Sil 50 g and eluted with Cy/EtOAc (3CV 100% Cy, 3CV from 100% to 95:5, 5CV 95:5).

Collected and evaporated fractions gave the title compound D113 as colourless oil (80 mg).

UPLC (IPQC): rt=1.50 minutes, peak observed: 499 (M+1) C₁₈H₂₂F₃₁N₂O₃ requires 498.

### Description 114: 1,1-dimethylethyl (1S,4S,6S)-4-({[4-methyl-5-(trifluoromethyl)-2-pyridinyl]oxy}methyl)-3-azabicyclo[4.1.0]heptane-3-carboxylate (D114)

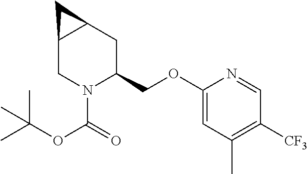

In a 40 ml screw-capped vial, 1,1-dimethylethyl (1S,4S,6S)-4-({[4-iodo-5-(trifluoromethyl)-2-pyridinyl]oxy}methyl)-3-azabicyclo[4.1.0]heptane-3-carboxylate D113 (80 mg), methylboronic acid (11.53 mg, 0.193 mmol), Pd(PPh₃)₄ (9.28 mg, 8.03 mmol), cesium carbonate (157 mg, 0.482 mmol) were suspended in DME (6 ml) and stirred for 1 hour at 110° C.

After this time the reaction mixture was filtered through a celite pad, the vial was rinsed with EtOAc (20 ml) and it was used to wash the celite pad. The solvents were evaporated under vacuum to give a brown oil, which was charged on a SNAP KP-Sil 50 g, eluted with 13CV of Cy/EtOAc 95:5.

Collected and evaporated fractions gave the title compound D114 as yellow oil (60 mg).

UPLC (IPQC): rt=1.47 minutes, peak observed: 387 (M+1) C₁₉H₂₅F₃N₂O₃ requires 386.

### Description 115: (1S,4S,6S)-4-({[4-methyl-5-(trifluoromethyl)-2-pyridinyl]oxy}methyl)-3-azabicyclo[4.1.0]heptane (D115)

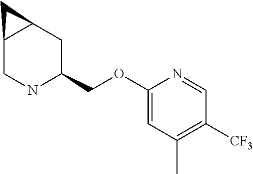

TFA (0.25 ml, 3.24 mmol) was added to an ice bath cooled solution of 1,1-dimethylethyl (1S,4S,6S)-4-({[4-methyl-5-(trifluoromethyl)-2-pyridinyl]oxy}methyl)-3-azabicyclo[4.1.0]heptane-3-carboxylate D114 (60 mg) in DCM (1 ml). The resulting mixture was stirred while the temperature reached 23° C.

The solvent was removed under reduced pressure and the brown oil obtained was charged on a SCX cartridge, washed with MeOH (24 ml) and eluted with 2M NH₃/MeOH (1.5 ml).

The ammoniacal fraction was evaporated under vacuum and the title compound D115 was obtained as yellowish oil (40 mg).

¹H NMR (400 MHz, DMSO-d₆) δ ppm 0.28-0.40 (m, 1H) 0.42-0.56 (m, 1H) 0.72-0.87 (m, 1H) 0.87-1.02 (m, 1H) 1.10-1.27 (m, 1H) 1.68 (br. s., 1H) 1.83-2.02 (m, 1 H) 2.39 (s, 3H) 2.60-2.73 (m, 1H) 2.91-3.02 (m, 1H) 3.02-3.09 (m, 1H) 3.98-4.14 (m, 2H) 6.90 (s, 1H) 8.41 (s, 1H)

### Description 116: 1,1-dimethylethyl (1S,4S,6S)-4-{[(4,6-dichloro-2-pyridinyl)oxy]methyl}-3-azabicyclo[4.1.0]heptane-3-carboxylate (D116A) and 1,1-dimethylethyl (1S,4S,6S)-4-{[(2,6-dichloro-4-pyridinyl)oxy]methyl}-3-azabicyclo[4.1.0]heptane-3-carboxylate (D116B)

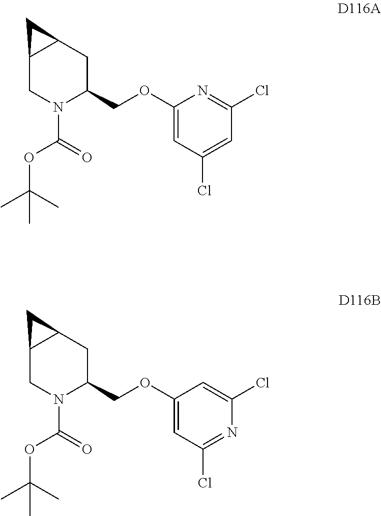

In a 40 ml screw-capped vial 1,1-dimethylethyl (1S,4S,6S)-4-(hydroxymethyl)-3-azabicyclo[4.1.0]heptane-3-carboxylate D104 (300 mg) and 2,4,6-trichloropyridine (241 mg, 1.320 mmol) were dissolved in DMF (15 ml) and NaH (68.6 mg, 1.716 mmol) was added at 0° C. The resulting mixture was stirred for 15 hours, during this time the temperature reached 23° C.

The reaction mixture was poured in a separatory funnel with saturated NaHCO₃ (40 ml), the vial was rinsed with Et₂O (15 ml) and water (40 ml) and the aqueous layer was backextracted with Et₂O (3×10 ml), the collected organic layers were washed with brine (4×5 ml), separated, dried over Na₂SO₄, filtered and evaporated under reduced pressure. The yellow oil obtained was charged on a SNAP KP-Sil 50 g and eluted with Cy/EtOAc (1 CV 100% Cy, 1 CV from 100% to 98:2, 3 CV 98:2, 1 CV from 98:2 to 96:4, 5 CV 96:4).

Collected and evaporated fractions gave the title compounds D116A (80 mg) as colourless oil and D116B (260 mg) like yellow oil.

### D116A:

UPLC (Basic GEN_QC: rt=1.21 minutes, peak observed: 373 (M+1) C₁₇H₂₂Cl₂N₂O₃ requires 372

### D116B:

UPLC (Basic GEN_QC: rt=1.11 minutes, peak observed: 373 (M+1) C₁₇H₂₂Cl₂N₂O₃ requires 372.

### Description 117: (1S,4S,6S)-4-{[(4,6-dichloro-2-pyridinyl)oxy]methyl}-3-azabicyclo[4.1.0]heptanes (D117)

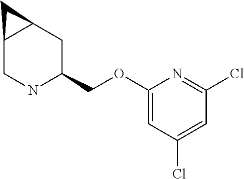

TFA (0.35 ml, 4.54 mmol) was added to an ice bath cooled solution of 1,1-dimethylethyl (1S,4S,6S)-4-{[(4,6-dichloro-2-pyridinyl)oxy]methyl}-3-azabicyclo[4.1.0]heptane-3-carboxylate D116A (80 mg) in DCM (1.4 ml). The resulting mixture was stirred while the temperature reached 23° C.

The solvent was removed under reduced pressure and the brown oil obtained was charged on a 1 g SCX cartridge, washed with MeOH (30 ml) and eluted with 2M NH₃/MeOH (1.5 ml).

The ammoniac fraction was evaporated under vacuum and the title compound D117 (46 mg) was obtained as yellowish oil.

¹H NMR (400 MHz, DMSO-d₆) δ ppm 0.20-0.39 (m, 1H) 0.40-0.54 (m, 1H) 0.74-0.87 (m, 1H) 0.87-1.01 (m, 1H) 1.11-1.29 (m, 1H) 1.83-1.98 (m, 1H) 2.58-2.71 (m, 1H) 2.91-3.02 (m, 1H) 3.05 (d, 1H) 3.89-4.00 (m, 1H) 4.00-4.08 (m, 1H) 7.03 (d, 1H) 7.32 (d, 1H)

### Description 118: (1S,4S,6S)-4-{[(2,6-dichloro-4-pyridinyl)oxy]methyl}-3-azabicyclo[4.1.0]heptanes (D118)

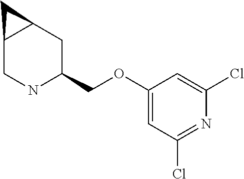

TFA (1.05 ml, 13.63 mmol) was added to an ice bath cooled solution of 1,1-dimethylethyl (1S,4S,6S)-4-{[(2,6-dichloro-4-pyridinyl)oxy]methyl}-3-azabicyclo[4.1.0]heptane-3-carboxylate D116B (240 mg) in DCM (4.2 ml). The resulting mixture was stirred while the temperature reached 23° C.

The solvent was removed under reduced pressure and the brown oil obtained was charged on a 2 g SCX cartridge, washed with MeOH (30 ml) and eluted with 2M NH₃/MeOH (7.5 ml).

The ammoniac fraction was evaporated under vacuum and the title compound D118 (175 mg) was obtained as yellowish oil.

¹H NMR (400 MHz, DMSO-d₆) d ppm 0.25-0.39 (m, 1H) 0.43-0.56 (m, 1H) 0.73-0.87 (m, 1H) 0.87-1.06 (m, 1H) 1.13-1.25 (m, 1H) 1.73 (br. s., 1H) 1.83-1.97 (m, 1 H) 2.63 (m, 1H) 2.90-3.10 (m, 2H) 3.76-3.88 (m, 1H) 3.91-4.04 (m, 1H) 7.14-7.24 (m, 2H)

### Description 119: 1,1-dimethylethyl (1S,4S,6S)-4-{[(4-chloro-2-pyridinyl)oxy]methyl}-3-azabicyclo[4.1.0]heptane-3-carboxylate (D119)

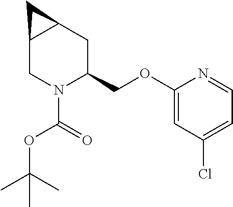

1,1-dimethylethyl (1S,4S,6S)-4-(hydroxymethyl)-3-azabicyclo[4.1.0]heptane-3-carboxylate D104 (100 mg), 4-chloro-2-pyridone (85 mg, 0.660 mmol), and tributylphosphane (0.163 ml, 0.660 mmol) were dissolved in THF (3 ml) into an 8 ml-capped vial under nitrogen (fine suspension).

TMAD (114 mg, 0.660 mmol) was added in one portion and the resulting mixture was stirred at room temperature.

After 2 hours an UPLC check showed an almost complete conversion.

The mixture was then partitioned between NaHCO₃ saturated solution (20 ml) and Et₂O (20 ml); water layer extracted with Et₂O (3×15 ml).

The organic phases were joined and dried over Na₂SO₄ and evaporated at reduced pressure.

The so obtained crude colorless oil (380 mg) was purified by Biotage Snap-120 g reverse phase (C18, eluent-A: water+0.1% HCOOH; eluent-B: ACN+0.1% HCOOH; from all A to all B).

After evaporation at reduced pressure of the pure collected fractions it was obtained the title compound D119 (50.6 mg) as colourless oil.

¹H NMR (500 MHz, DMSO-d₆) δ ppm 0.12-0.23 (m, 1H) 0.65-0.75 (m, 1H) 0.81-0.92 (m, 1H) 0.96-1.18 (m, 2H) 1.37 (s, 9H) 2.22-2.31 (m, 1H) 2.34-2.45 (m, 1H) 3.86-3.94 (m, 1H) 4.10-4.40 (m, 3H) 6.96 (d, 1H) 7.09 (dd, 1H) 8.11 (d, 1H)

### Description 120: (1S,4S,6S)-4-{[(4-chloro-2-pyridinyl)oxy]methyl}-3-azabicyclo[4.1.0]heptane (D120)

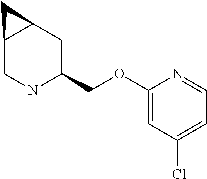

1,1-dimethylethyl (1S,4 S,6S)-4-{[(4-chloro-2-pyridinyl)oxy]methyl}-3-azabicyclo[4.1.0]heptane-3-carboxylate D119 (50 mg) was dissolved in a TFA (0.5 ml, 6.49 mmol) solution in DCM (3 ml) under nitrogen and the so obtained mixture was stirred at room temperature for 1.5 hours.

The whole mixture was loaded onto an SCX-2 G column, firstly eluted with DCM (5 ml), then MeOH (10 ml): the target material was then recovered eluting with NH₃ 2N in MeOH (10 ml).

After evaporation at reduced pressure of the ammoniacal solution it was obtained the title compound D120 (34.5 mg) as colourless oil.

UPLC (IPQC): rt=0.56 minutes, peak observed: 239 (M+1) C₁₂H₁₅ClN₂O requires 238.

### Description 121: 1,1-dimethylethyl (1S,4S,6S)-4-({[5-(trifluoromethyl)-2-pyrazinyl]oxy}methyl)-3-azabicyclo[4.1.0]heptane-3-carboxylate (D121)

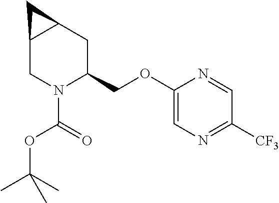

To a solution of 1,1-dimethylethyl (1R,4S,6R)-4-(hydroxymethyl)-3-azabicyclo[4.1.0]heptane-3-carboxylate D104 (120 mg) and 2-bromo-5-(trifluoromethyl)pyrazine D66 (120 mg) in DMF (2 ml) at 0° C. (ice bath) was added NaH (31.7 mg, 0.792 mmol) (gas evolution). The reaction mixture was slowly warmed to room temperature and stirred at room temperature for 1 hour. The reaction was quenched by a slow and careful addition of saturated aqueous solution of NaHCO₃ (40 ml). The organic phase was extracted with DCM (3×20 ml), the combined organic phases were washed with water and brine, dried over Na₂SO₄, filtered and concentrated to give the title compound D121 (150 mg) which was used in the next step without any purification.

UPLC (Acid Final_QC): rt=0.89 minutes, peak observed: 274 (M+1-Boc), 374 (M+1) C₁₇H₂₂F₃N₃O₃ requires 373.

### Description 122: (1S,4S,6S)-4-({[5-(trifluoromethyl)-2-pyrazinyl]oxy}methyl)-3-azabicyclo[4.1.0]heptane (D122)

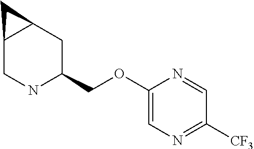

1,1-dimethylethyl (1S,4S,6S)-4-({[5-(trifluoromethyl)-2-pyrazinyl]oxy}methyl)-3-azabicyclo[4.1.0]heptane-3-carboxylate D121 (150 mg of the crude obtained in description 124) was dissolved in DCM (2 ml) then TFA (1 ml, 12.98 mmol) was added and the mixture was stirred for 3 hours at room temperature. All volatiles were removed under vacuum and the residue was purified by Silica —NH Chromatography (Biotage SP—Column size 28 g, DCM to DCM/MeOH 9:1 as eluent). It was recovered the title compound D122 (25 mg).

UPLC (Acid Final_QC): rt=0.77 minutes, peak observed: 471 (M+1) C₂₃H₂₁F₃N₆O₂ requires 470.

¹H NMR (500 MHz, DMSO-d₆) δ ppm 0.19-0.25 (m, 1H) 0.64-0.73 (m, 1H) 0.96-1.39 (m, 3H) 2.48-2.52 (m, 1H) 2.54 (s, 3H) 2.64-2.70 (m, 1H) 3.57-3.67 (m, 1H) 4.33-4.40 (m, 1H) 4.49-4.57 (m, 1H) 4.63-4.71 (m, 1H) 7.37-7.51 (m, 2H) 8.45-8.51 (m, 2H) 8.75-8.82 (m, 3H)

### Description 123: 3-(2H-1,2,3-triazol-2-yl)-2-pyridinecarbonitrile (D123)

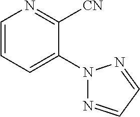

DMF (12 ml) was added to a mixture of 3-bromo-2-pyridinecarbonitrile (1.18 g, 6.45 mmol), 1H-1,2,3-triazole (0.748 ml, 12.90 mmol), (1R,2R)—N,N′-dimethyl-1,2-cyclohexanediamine (0.183 g, 1.290 mmol), copper(I)trifluoromethanesulfonate benzene complex (0.162 g, 0.322 mmol) and cesium carbonate (4.20 g, 12.90 mmol). The mixture was degassed via 3 vacuum/nitrogen cycles and heated in microwave at 120° C. for 45 minutes. Water was added and the aqueous extracted with EtOAc, the phases were separated on a hydrophobic filter and the combined organic solvent was removed to give the crude product. This was purified by a silica gel chromatography (100 g column, eluted with cyclohexane/EtOAc 0 to 40%) to give a first batch of the title compound D123 (896 mg) as a white solid.

Material recovered from the column containing desired product (300 mg) was purified another time (silica gel chromatography 25 g column, eluted with cyclohexane/EtOAc 0 to 35%) to give a second batch of the title compound D123 (100 mg) as a white solid.

UPLC (Basic GEN_QC): rt=0.58 minutes, peak observed: 172 (M+1) C₈H₅N₅ requires 171.

### Description 124: 3-(2H-1,2,3-triazol-2-yl)-2-pyridinecarboxylic acid (D124)

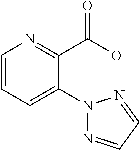

To a solution of 3-(2H-1,2,3-triazol-2-yl)-2-pyridinecarbonitrile D123 (996 mg, 5.82 mmol) in EtOH (8 ml), a 3M solution of NaOH (9.70 ml, 29.1 mmol) in water (4.00 ml) was added in one portion. The mixture was stirred 15 hours at 100° C. using a PLS apparatus. The solvent was evaporated at reduced pressure, obtaining the desired acid as sodium salt.

This material was adjusted to pH=4 with HCl solution. The so obtained solution was loaded onto a pre-conditioned C18 column (70 g). The column was eluted with water and then MeOH. Some aqueous fractions showed to contain the desired acid, so were evaporated under reduced pressure to give a first batch of the title compound D124 (340 mg) as white solid. Also the first four MeOH fractions showed to contain the desired product so they were evaporated under reduced pressure to give a second batch of title compound D124 (367 mg).

¹H NMR (400 MHz, DMSO-d₆) δ ppm 7.34 (dd, 2H) 7.91 (dd, 2H) 8.46 (dd, 2H)

### Examples

### Example 57

### (1S,4S,6S)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane (E57)

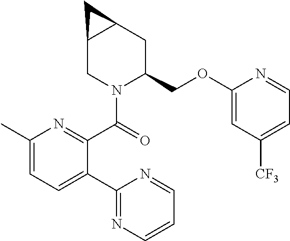

6-methyl-3-(2-pyrimidinyl)-2-pyridinecarboxylic acid D59 (125 mg) was suspended in DCM (2 ml), then (1S,4S,6S)-4-({[4-(trifluoromethyl)-2-pyridinyl]oxy}methyl)-3-azabicyclo[4.1.0]heptane D107 (50 mg) was added followed by DIPEA (65 μl, 0.372 mmol): the mixture was stirred at room temperature for 10 minutes.

TBTU (65 mg, 0.202 mmol) was added in one portion and the mixture was stirred at room temperature for 2 hours.

The whole mixture was loaded onto an SCX-5 G column, firstly eluted with DCM (10 ml), then MeOH (10 ml): the target material was then collected eluting with NH₃ 2N in MeOH (20 ml).

After evaporation at reduced pressure of the ammoniacal solution it was obtained the crude target material as pale yellow oil (133 mg).

This was purified twice by Biotage (1] Snap-25 G silica gel column, EtOAc/Cy from pure Cy to 80:20; 2] Snap-25 G silica gel column, DCM/MeOH 98:02).

After evaporation at reduced pressure of the pure collected fractions it was obtained the title compound E57 as white solid (39.5 mg).

UPLC (IPQC): rt=1.17 minutes peak observed: 470 (M+1) C₂₄H₂₂F₃N₅O₂ requires 469

¹H NMR (500 MHz, DMSO-d₆) δ ppm 0.18-0.29 (m, 1H) 0.59-0.75 (m, 1H) 0.94-1.03 (m, 1H) 1.03-1.13 (m, 1H) 1.27-1.42 (m, 1H) 2.40-2.47 (m, 1H) 2.56 (s, 3H) 2.60-2.72 (m, 1H) 3.55-3.65 (m, 1H) 4.28-4.39 (m, 1H) 4.38-4.47 (m, 1H) 4.53-4.65 (m, 1H) 7.20-7.26 (m, 1H) 7.34-7.37 (m, 1H) 7.37-7.43 (m, 1H) 7.44-7.49 (m, 1H) 8.44-8.53 (m, 2H) 8.76 (d, 2H)

### Example 58

### (1S,4S,6S)-3-{[3-(2-pyrimidinyl)-2-pyridinyl]carbonyl}-4-({[4-(trifluoromethyl)-2-pyridinyl]oxy}methyl)-3-azabicyclo[4.1.0]heptane (E59)

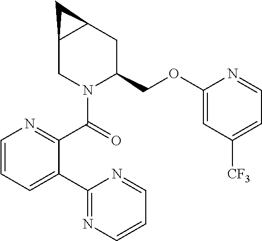

3-(2-pyrimidinyl)-2-pyridinecarboxylic acid D105 (28 mg) was suspended in DCM (2 ml), then (1S,4S,6S)-4-({[4-(trifluoromethyl)-2-pyridinyl]oxy}methyl)-3-azabicyclo[4.1.0]heptane D107 (35 mg) was added followed by DIPEA (45 μl, 0.258 mmol): the mixture was stirred at room temperature for 10 minutes.

TBTU (45 mg, 0.140 mmol) was added in one portion and the mixture was stirred at room temperature for 2 hours.

The whole mixture was loaded onto an SCX-5 G column, firstly eluted with DCM (10 ml), then MeOH (10 ml): the target material was then collected eluting with NH₃ 2N in MeOH (20 ml).

After evaporation at reduced pressure of the ammoniacal solution it was obtained the crude target material as pale yellow oil (82 mg).

This was purified by Biotage (Snap-11 G NH-column, EtOAc/Cy from pure Cy to 90:10).

After evaporation at reduced pressure of the pure collected fractions it was obtained the title compound E58 as white solid (41 mg).

UPLC (Basic GEN_QC: rt=0.90 minutes, peak observed; 456 (M+1) C₂₃H₂₀F₃N₅O₂ requires 455.

¹H NMR (400 MHz, DMSO-d₆) δ ppm 0.17-0.29 (m, 1H) 0.59-0.75 (m, 1H) 0.91-1.06 (m, 1H) 1.04-1.21 (m, 1H) 1.22-1.41 (m, 1H) 2.38-2.55 (m, 1H) 2.64-2.79 (m, 1H) 3.59-3.70 (m, 1H) 4.28-4.38 (m, 1H) 4.39-4.60 (m, 2H) 7.22 (none, 1H) 7.35 (d, 1H) 7.43 (t, 1H) 7.60-7.65 (m, 1H) 8.46 (d, 1H) 8.58 (d, 1H) 8.67-8.71 (m, 1H) 8.79 (d, 2 H)

### Example 59

### (1S,4S,6S)-3-{[5-methyl-2-(2-pyrimidinyl)phenyl]carbonyl}-4-({[4-(trifluoromethyl)-2-pyridinyl]oxy}methyl)-3-azabicyclo[4.1.0]heptane (E59)

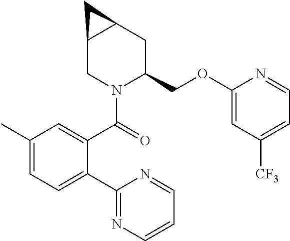

(1S,4S,6S)-4-({[4-(trifluoromethyl)-2-pyridinyl]oxy}methyl)-3-azabicyclo[4.1.0]heptane D107 (21 mg) was dissolved in DCM (1 ml) into a 8 ml capped-vial under nitrogen, then 5-methyl-2-(2-pyrimidinyl)benzoic acid (21.5 mg, 0.100 mmol) was added followed by DIPEA (0.035 ml, 0.201 mmol) and T₃P (50% in EtOAc) (0.16 ml, 0.269 mmol) (the mixture became light yellow): the mixture was stirred at 45° C. for 4.5 hours using a PLS apparatus.

The orange mixture was partitioned between DCM (10 ml) and NaOH (1M water solution) and the water phase extracted with DCM (2×10 ml).

The organic fractions were joined together, dried over Na₂SO₄ and evaporated at reduced pressure, obtaining the crude target material as yellow oil (49.5 mg).

This was purified by Biotage (Snap-11 G NH-column, EtOAc/Cy from pure Cy to 50:50).

After evaporation at reduced pressure of the pure collected fractions it was obtained the title compound E59 as pale yellow solid (29.2 mg).

UPLC (Basic GEN_QC: rt=1.04 minutes, peak observed; 469 (M+1) C₂₅H₂₃F₃N₄O₂ requires 468

¹H NMR (500 MHz, DMSO-d₆) δ ppm 0.11-0.19 (m, 1H) 0.59-0.64 (m, 1H) 0.73-0.84 (m, 1H) 0.87-0.98 (m, 1H) 1.18-1.34 (m, 1H) 2.37-2.48 (m, 1H) 2.50 (s, 3H) 2.59-2.76 (m, 1H) 3.58-3.76 (m, 2H) 4.22-4.36 (m, 1H) 4.34-4.46 (m, 1H) 6.69 (s, 1H) 7.06-7.45 (m, 4H) 8.12 (d, 1H) 8.43 (d, 1H) 8.82 (d, 2H).

### Example 60

### (1S,4S,6S)-4-{2-[(5-fluoro-2-pyridinyl)oxy]ethyl}-3-{[6-methyl-3-(2H-1,2,3-triazol-2-yl)-2-pyridinyl]carbonyl}-3-azabicyclo[4.1.0]heptane (E60)

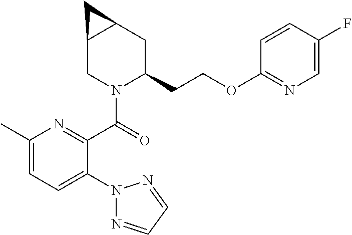

TBTU (29.9 mg, 0.093 mmol) was added to a stirred solution of (1S,4S,6S)-4-{2-[(5-fluoro-2-pyridinyl)oxy]ethyl}-3-azabicyclo[4.1.0]heptane D112 (20 mg), 6-methyl-3-(2H-1,2,3-triazol-2-yl)-2-pyridinecarboxylic acid D33 (17.28 mg) and DIPEA (0.018 ml, 0.102 mmol) in DCM (3 ml) at room temperature. The reaction was stirred for 3 hours then quenched with saturated NaHCO₃ aqueous solution (30 ml), and extracted with EtOAc (2×20 ml). The combined organic phases were washed with water (20 ml), brine (20 ml), dried (Na₂SO₄) and evaporated under reduced pressure to give a colourless residue which was purified via Biotage (30-80% EtOAc/cyclohexane, 2 SNAP 11 NH columns in series) to give the title compound E60 (29 mg) as colourless gum.

UPLC (Basic GEN_QC): rt1=0.82 minutes and rt2=0.86 minutes (rotamers present) peak observed: 423 (M+1) C₂₂H₂₃FN₆O₂ requires 422

¹H NMR (500 MHz, DMSO-d₆) δ ppm 0.15-0.22 (m, 1H) 0.57-0.66 (m, 1H) 0.74-0.96 (m, 2H) 1.04-1.15 (m, 1H) 1.79-1.89 (m, 1H) 2.05-2.15 (m, 1H) 2.30-2.37 (m, 1H) 2.48-2.52 (m, 1H) 2.54 (s, 3H) 3.41-3.50 (m, 1H) 4.04-4.13 (m, 1H) 4.26 (t, 2H) 6.44-6.47 (m, 1H) 6.87 (d, 1H) 7.48 (d, 1H) 7.62-7.64 (m, 1H) 7.65-7.70 (m, 1H) 8.00 (d, 1H) 8.09 (d, 1H) 8.11-8.15 (m, 1H)

### Example 61

### (1S,4S,6S)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane (E61)

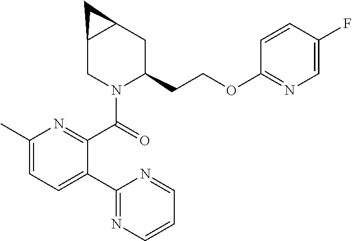

TBTU (59.8 mg, 0.186 mmol) was added to a stirred solution of (1S,4S,6S)-4-{2-[(5-fluoro-2-pyridinyl)oxy]ethyl}-3-azabicyclo[4.1.0]heptane D112 (40 mg), 6-methyl-3-(2-pyrimidinyl)-2-pyridinecarboxylic acid D59 (110 mg) and DIPEA (0.044 ml, 0.254 mmol) in DCM (3 ml) at room temperature. The reaction was stirred for 1 hour and 90 minutes then quenched with saturated NaHCO₃ aqueous solution (30 ml) and extracted with EtOAc (2×30 ml). The combined organic phases were washed with water (20 ml), brine (20 ml), dried (Na₂SO₄) and evaporated under reduced pressure to give a red residue which was purified via Biotage (30-100% EtOAc/cyclohexane, 2 SNAP 11 NH columns in series) to give 43 mg of a colourless gum.

This was loaded onto a preconditioned SCX cartridge (2 g) and eluted with MeOH and then 2M NH₃ in MeOH. The basic fractions were evaporated under reduced pressure to give the title compound E61 (41 mg) as white gummy solid.

UPLC (Basic GEN_QC: rt1=0.80 minutes and rt2=0.85 minutes (rotamers present) peak observed: 434 (M+1) C₂₄H₂₄FN₅O₂ requires 433

¹H NMR (500 MHz, DMSO-d₆) δ ppm 0.18-0.26 (m, 1H) 0.59-0.69 (m, 1H) 0.74-0.87 (m, 1H) 1.06-1.34 (m, 3H) 1.86-1.98 (m, 1H) 2.14-2.25 (m, 1H) 2.50 (s, 3H) 2.57-2.64 (m, 1H) 3.51-3.66 (m, 1H) 4.06-4.22 (m, 1H) 4.26-4.42 (m, 2H) 6.81-6.93 (m, 1 H) 7.37-7.49 (m, 2H) 7.60-7.74 (m, 1H) 8.09-8.18 (m, 1H) 8.45 (d, 1H) 8.82 (d, 2H)

### Example 62

### (1S,4S,6S)-4-{2-[(5-fluoro-2-pyridinyl)oxy]ethyl}-3-{[6-methyl-3-(1H-pyrazol-1-yl)-2-pyridinyl]carbonyl}-3-azabicyclo[4.1.0]heptane (E62)

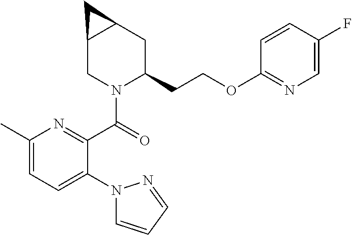

TBTU (44.8 mg, 0.140 mmol) was added to a stirred solution of (1S,4S,6S)-4-{2-[(5-fluoro-2-pyridinyl)oxy]ethyl}-3-azabicyclo[4.1.0]heptane D112 (30 mg), 6-methyl-3-(1H-pyrazol-1-yl)-2-pyridinecarboxylic acid D37 (25.8 mg) and DIPEA (0.033 ml, 0.190 mmol) in DCM (3 ml) at room temperature. The reaction was stirred for 2 hours, then quenched with saturated NaHCO₃ solution (30 ml) and extracted with EtOAc (2×20 ml). The combined organic phases were washed with water (20 ml), brine (20 ml), dried (Na₂SO₄) and evaporated under reduced pressure to give a colourless residue which was purified via Biotage (20-70% EtOAc/cyclohexane, 2 SNAP 11 NH columns in series) to give the title compound E62 (50 mg) as a colourless gum.

UPLC (Basic GEN_QC: rt1=0.82 minutes and rt2=0.86 minutes (rotamers present) peak observed: 422 (M+1) C₂₃H₂₄FN₅O₂ requires 421.

¹H NMR (500 MHz, DMSO-d₆) δ ppm 0.15-0.22 (m, 1H) 0.57-0.66 (m, 1H) 0.74-0.96 (m, 2H) 1.04-1.15 (m, 1H) 1.79-1.89 (m, 1H) 2.05-2.15 (m, 1H) 2.30-2.37 (m, 1H) 2.48-2.52 (m, 1H) 2.54 (s, 3H) 3.41-3.50 (m, 1H) 4.04-4.13 (m, 1H) 4.26 (t, 2H) 6.44-6.47 (m, 1H) 6.87 (d, 1H) 7.48 (d, 1H) 7.62-7.64 (m, 1H) 7.65-7.70 (m, 1H) 8.00 (d, 1H) 8.09 (d, 1H) 8.11-8.15 (m, 1H)

### Example 63

### (1S,4S,6S)-3-{[6-methyl-3-(2-pyrimidinyl)-2-pyridinyl]carbonyl}-4-({[4-methyl-5-(trifluoromethyl)-2-pyridinyl]oxy}methyl)-3-azabicyclo[4.1.0]heptane (E63)

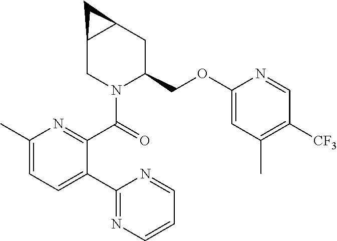

To a solution of (1S,4S,6S)-4-({[4-methyl-5-(trifluoromethyl)-2-pyridinyl]oxy}methyl)-3-azabicyclo[4.1.0]heptane D115 (40 mg) in DCM (1.7 ml) were added 6-methyl-3-(2-pyrimidinyl)-2-pyridinecarboxylic acid D59 (100 mg) and DIPEA (0.073 ml, 0.419 mmol). To the yellowish suspension TBTU (52.0 mg, 0.162 mmol) was added and the reaction mixture was stirred at 23° C. for 1 hour.

Saturated NaHCO₃ (2 ml) was added to the reaction mixture and it was transferred in a separatory funnel. The flask was rinsed with DCM (2 ml) and water (2 ml).

After the separation, the aqueous layer was backextracted with DCM (2×2 ml).

The combined organic layers were washed with water (4×2 ml), dried over Na₂SO₄, filtered and evaporated under reduced pressure. An orange oil was obtained and it was purified by silica gel chromatography (SNAP KP—NH 11 g; eluted with Cy/iPrOH 1 CV 100% Cy, 2 CV from 100% to 99:1, 3 CV 99:1, 2 CV from 99:1 to 98:2, 5 CV 98:2, 2 CV from 98:2 to 97:3, 5 CV 97:3).

The evaporation of the fractions gave the title compound E63 (38 mg).

UPLC (Basic GEN_QC): rt=0.99 minutes, peak observed: 484 (M+1) C₂₅H₂₄F₃N₅O₂ requires 483.

¹H NMR (500 MHz, DMSO-d₆) δ ppm 0.14-0.24 (m, 1H) 0.62-0.76 (m, 1H) 0.91-1.14 (m, 2H) 1.19-1.36 (m, 2H) 2.41 (s, 3H) 2.56 (s, 3H) 2.59-2.69 (m, 1H) 3.55-3.68 (m, 1H) 4.28-4.36 (m, 1H) 4.36-4.43 (m, 1H) 4.55-4.62 (m, 1H) 6.95 (s, 1H) 7.41 (t, 1H) 7.44-7.50 (m, 1H) 8.44-8.52 (m, 2H) 8.74-8.83 (m, 2H)

### Example 64

### (1S,4S,6S)-3-{[6-methyl-3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane (E64)

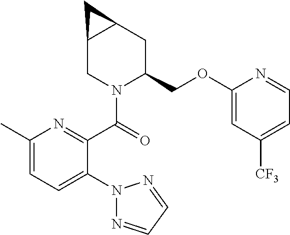

6-methyl-3-(2H-1,2,3-triazol-2-yl)-2-pyridinecarboxylic acid D33 (28 mg) was suspended in DCM (2 ml), then (1S,4S,6S)-4-({[4-(trifluoromethyl)-2-pyridinyl]oxy}methyl)-3-azabicyclo[4.1.0]heptane D107 (35 mg) was added followed by DIPEA (45 μl, 0.258 mmol): the mixture was stirred at room temperature for 10 minutes. TBTU (45 mg, 0.140 mmol) was added in one portion and the mixture was stirred at room temperature for 2 hours.

The whole mixture was loaded onto an SCX-5 G column, firstly eluted with DCM (10 ml), then MeOH (10 ml): the target material was then collected eluting with NH₃ 2N in MeOH (20 ml).

After evaporation at reduced pressure of the ammoniacal solution it was obtained the crude target material as pale yellow oil (86 mg).

This material was purified by Biotage (Snap-11 G NH-column, EtOAc/Cy from pure Cy to 50:50).

After evaporation at reduced pressure of the pure collected fractions it was obtained the title compound E64 (48 mg) as white solid.

UPLC (Basic GEN_QC): 0.96 minutes, peak observed: 459 (M+1) C₂₂H₂₁F₃N₆O₂ requires 458.

¹H NMR (400 MHz, DMSO-d₆) δ ppm 0.21-0.33 (m, 1H) 0.64-0.74 (m, 1H) 0.78-1.15 (m, 2H) 1.30-1.46 (m, 1H) 2.37-2.49 (m, 1H) 2.56 (s, 3H) 2.63-2.76 (m, 1H) 3.58-3.69 (m, 1H) 4.20-4.34 (m, 1H) 4.35-4.46 (m, 1H) 4.46-4.56 (m, 1H) 7.22 (s, 1H) 7.35 (d, 1H) 7.53 (d, 1H) 7.79-7.88 (m, 2H) 8.23 (d, 1H) 8.46 (d, 1H)

The following compounds were prepared using a similar procedure to that described for Example 58-64 (in some examples the solvent used was DCM instead of DMF). Each compound was obtained by amide coupling of (1S,4S,6S)-4-{[(heteroaryl)oxy]methyl}-3-azabicyclo[4.1.0]heptane with the appropriate carboxylic acid. This is provided merely for assistance to the skilled chemist. The starting material may not necessarily have been prepared from the batch referred to.

| | Amide coupling | |
|---|---|---|
| No. | Reactants | Characterising data |
| 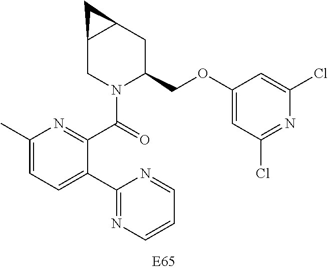 | D120 and D59 | (1S,4S,6S)-4-{[(2,6-dichloro-4- pyridinyl)oxy]methyl}-3-{[6-methyl-3-(2- pyrimidinyl)-2-pyridinyl]carbonyl}-3- azabicyclo[4.1.0]heptane UPLC (Basic GEN_QC): 0.93 minutes, peak observed: 470 (M + 1) C₂₃H₂₁Cl₂N₅O₂ requires 469 ¹H NMR (500 MHz, DMSO-d₆) δ ppm −0.05-0.04 (m, 1 H) 0.41-0.51 (m, 1 H) 0.68-0.90 (m, 2 H) 0.96-1.15 (m, 1 H) 2.17-2.29 (m, 1 H) 2.35 (s, 3 H) 2.38- 2.49 (m, 1 H) 3.35-3.44 (m, 1 H) 3.99- 4.08 (m, 1 H) 4.08-4.27 (m, 2 H) 7.13 (s, 2 H) 7.21 (t, 1 H) 7.27 (d, 1 H) 8.29 (d, 1 H) 8.53-8.63 (m, 2 H) |
| 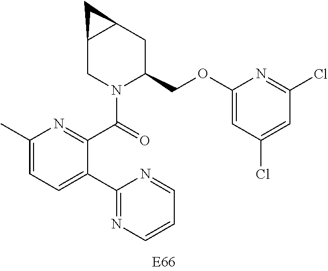 | D117 and D59 | (1S,4S,6S)-4-{[(4,6-dichloro-2- pyridinyl)oxy]methyl}-3-{[6-methyl-3-(2- pyrimidinyl)-2-pyridinyl]carbonyl}-3- azabicyclo[4.1.0]heptane UPLC (Basic GEN_QC): 1.03 minutes, peak observed: 470 (M + 1) C₂₃H₂₁Cl₂N₅O₂ requires 469 ¹H NMR (400 MHz, DMSO-d₆) δ ppm 0.17-0.27 (m, 1 H) 0.62-0.75 (m, 1 H) 0.93-1.03 (m, 1 H) 1.03-1.15 (m, 1 H) 1.17-1.37 (m, 2 H) 2.56 (s, 3 H) 2.59- 2.69 (m, 1 H) 3.52-3.68 (m, 1 H) 4.25- 4.41 (m, 2 H) 4.50-4.59 (m, 1 H) 7.08- 7.14 (m, 1 H) 7.34-7.40 (m, 1 H) 7.41- 7.50 (m, 2 H) 8.50 (d, 1 H) 8.83 (d, 2 H) |
| 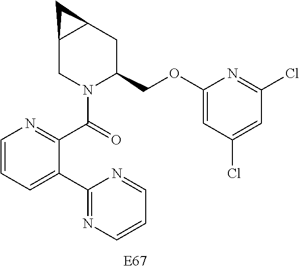 | D117 and D105 | (1S,4S,6S)-4-{[(4,6-dichloro-2- pyridinyl)oxy]methyl}-3-{[3-(2- pyrimidinyl)-2-pyridinyl]carbonyl}-3- azabicyclo[4.1.0]heptane UPLC (Basic GEN_QC): rt = 0.97 minutes, peak observed: 456 (M + 1) C₂₂H₁₉Cl₂N₅O₂ requires 455. ¹H NMR (400 MHz, DMSO-d₆) δ ppm 0.13-0.29 (m, 1 H) 0.62-0.79 (m, 1 H) 0.95-1.08 (m, 1 H) 1.08-1.18 (m, 1 H) 1.18-1.40 (m, 1 H) 2.41-2.58 (m, 1 H) 2.59-2.73 (m, 1 H) 3.54-3.68 (m, 1 H) 4.23-4.37 (m, 2 H) 4.43-4.57 (m, 1 H) 7.10 (d, 1 H) 7.37 (d, 1 H) 7.48 (t, 1 H) 7.60-7.66 (m, 1 H) 8.59 (dd, 1 H) 8.70 (dd, 1 H) 8.87 (d, 2 H) |
| 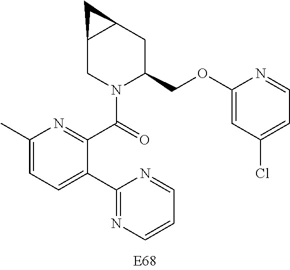 | D120 and D59 | (1S,4S,6S)-4-{[(4-chloro-2- pyridinyl)oxy]methyl}-3-{[6-methyl-3-(2- pyrimidinyl)-2-pyridinyl]carbonyl}-3- azabicyclo[4.1.0]heptane UPLC (Basic GEN_QC): rt = 0.90 minutes, peak observed: 437 (M + 1) C₂₃H₂₂ClN₅O₂ requires 436. ¹H NMR (500 MHz, DMSO-d₆) δ ppm 0.16-0.25 (m, 1 H) 0.62-0.71 (m, 1 H) 0.91-1.03 (m, 1 H) 1.02-1.15 (m, 1 H) 1.20-1.38 (m, 1 H) 2.42-2.51 (m, 1 H) 2.56 (s, 3 H) 2.61-2.69 (m, 1 H) 3.55- 3.65 (m, 1 H) 4.26-4.38 (m, 2 H) 4.53 (d, 1 H) 7.02 (s, 1 H) 7.14 (d, 1 H) 7.38-7.43 (m, 1 H) 7.47 (d, 1 H) 8.19 (d, 1 H) 8.49 (d, 1 H) 8.76 (d, 2 H) |
| 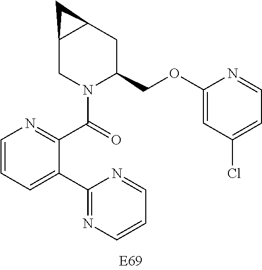 | D120 and D105 | (1S,4S,6S)-4-{[(4-chloro-2- pyridinyl)oxy]methyl}-3-{[3-(2- pyrimidinyl)-2-pyridinyl]carbonyl}-3- azabicyclo[4.1.0]heptane UPLC (Basic GEN_QC): 0.84 minutes, peak observed: 422 (M + 1) C22H20ClN5O2 requires 421. ¹H NMR (400 MHz, DMSO-d₆) δ ppm 0.17-0.26 (m, 1 H) 0.62-0.74 (m, 1 H) 0.94-1.05 (m, 1 H) 1.07-1.17 (m, 1 H) 1.22-1.40 (m, 1 H) 2.41-2.48 (m, 1 H) 2.62-2.73 (m, 1 H) 3.63 (dd, 1 H) 4.25- 4.40 (m, 2 H) 4.47-4.54 (m, 1 H) 7.02 (d, 1 H) 7.13 (dd, 1 H) 7.45 (t, 1 H) 7.57- 7.65 (m, 1 H) 8.19 (d, 1 H) 8.55-8.61 (m, 1 H) 8.68-8.72 (m, 1 H) 8.80 (d, 2 H) |
| 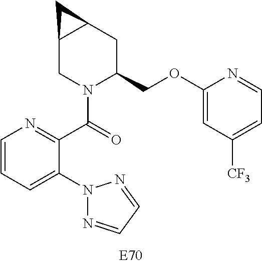 | D107 and D124 | (1S,4S,6S)-3-{[3-(2H-1,2,3-triazol-2-yl)-2- pyridinyl]carbonyl}-4-({[4- (trifluoromethyl)-2- pyridinyl]oxy}methyl)-3- azabicyclo[4.1.0]heptane UPLC (Basic GEN_QC): rt = 0.92 minutes, peak observed: 445 (M + 1) C₂₁H₁₉F₃N₆O₂ requires 444. ¹H NMR (500 MHz, DMSO-d₆) δ ppm −0.04-0.03 (m, 1 H) 0.36-0.49 (m, 1 H) 0.63-0.74 (m, 1 H) 0.74-0.84 (m, 1 H) 1.06-1.19 (m, 1 H) 2.12-2.23 (m, 1 H) 2.41-2.52 (m, 1 H) 3.33-3.45 (m, 1 H) 3.97-4.10 (m, 1 H) 4.10-4.31 (m, 2 H) 6.96 (s, 1 H) 7.09 (d, 1 H) 7.38-7.48 (m, 1 H) 7.56-7.69 (m, 2 H) 8.12 (d, 1 H) 8.20 (d, 1 H) 8.41 (d, 1 H) |
| 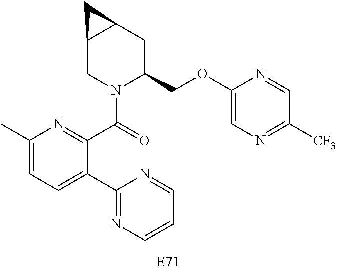 | D122 and D59 | (1S,4S,6S)-3-{[6-methyl-3-(2- pyrimidinyl)-2-pyridinyl]carbonyl}-4- ({[5-(trifluoromethyl)-2- pyrazinyl]oxy}methyl)-3- azabicyclo[4.1.0]heptane UPLC (Acid Final QC): rt = 0.77 minutes, peak observed: 471 (M + 1) C₂₃H₂₁F₃N₆O₂ requires 470. ¹H NMR (500 MHz, DMSO-d₆) δ ppm 0.19-0.25 (m, 1 H) 0.64-0.73 (m, 1 H) 0.96-1.39 (m, 3 H) 2.48-2.52 (m, 1 H) 2.54 (s, 3 H) 2.64-2.70 (m, 1 H) 3.57- 3.67 (m, 1 H) 4.33-4.40 (m, 1 H) 4.49- 4.57 (m, 1 H) 4.63-4.71 (m, 1 H) 7.37- 7.51 (m, 2 H) 8.45-8.51 (m, 2 H) 8.75- 8.82 (m, 3 H) |

### Example 72

### Determination of Antagonist Affinity at Human Orexin-1 and 2 Receptors Using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no. 345810). Cells were grown as monolayers under 95%:5% air:CO₂ at 37° C.

The sequences of the human orexin 1, human orexin 2, rat orexin 1 and rat orexin 2 receptors used in this example were as published in Sakurai, T. et al (1998) Cell, 92 pp 573 to 585. Some of the compounds of the invention were tested against a human orexin 1 receptor which had the sequence as published in Sakurai, T. et al supra with the exception that the amino acid residue at position 280 was alanine and not glycine.

### Measurement of [Ca²]ᵢ Using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 2 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵M to 1.58×10⁻¹¹M. Compounds of the invention were dissolved in dimethylsulfoxide (DMSO) at a stock concentration of 10 mM. These stock solutions were serially diluted with DMSO and 1 μL of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²]ᵢ using the fluometric imaging plate reader (FLIPR). In: Lambert DG (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 4-5 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of examples 1 to 71 were tested according to the method of example 72. All compounds gave an fpKi value of 5.5 or above at one or both of the orexin 1 or orexin 2 receptors. The compounds gave fpKi values from 5.5 to 9.7 at the human cloned orexin-1 receptor (as published in Sakurai, T. et al supra or as published in Sakurai, T. et al supra but having the amino acid residue alanine at position 280 and not glycine) and from 5.9 to 9.2 the human cloned orexin-2 receptor (with the exception of compound E4 which was <4.8).

1. A compound of formula (I) 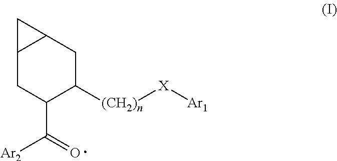
where
X is O or S;
n is 1 or 2;
Ar₁ is a 5 or 6-membered monocyclic aromatic group having 0, 1, 2 or 3 nitrogen atoms, which group is optionally substituted with 1 or 2 groups independently selected from C₁₋₄alkyl, C₁₋₄alkoxy, haloC₁₋₄alkyl, haloC₁₋₄alkoxy, halo or cyano; or Ar1 is an 8 to 10 membered bicyclic heterocyclyl group having 1, 2 or 3 heteroatoms selected from N, O or S which bicyclic heterocyclyl group is optionally substituted with C₁₋₄alkyl, haloC₁₋₄alkyl or halo;
Ar₂ is a group selected from phenyl, pyridinyl, pyrimidinyl, pyridazinyl, pyrazinyl or thiazolyl which group is substituted with 1 or 2 groups independently selected from C₁₋₄alkyl, C₁₋₄alkoxy, haloC₁₋₄alkyl, haloC₁₋₄alkoxy, cyano or a group Y;
Y is a group selected from phenyl, phenyloxy, pyridyl, pyrimidinyl, pyridazinyl, pyrazinyl, oxadiazolyl or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;
or a pharmaceutically acceptable salt thereof.

2. A compound according to claim 1 wherein the compound is in a trans (1R,4S,6R)-configuration.

3. A compound according to claim 1 or claim 2 where X is O, or a pharmaceutically acceptable salt thereof.

4. A compound according to any one of claims 1 to 3 where Ar₁ is pyridinyl, or a pharmaceutically acceptable salt thereof.

5. A compound according to any one of claims 1 to 4 where Ar₂ is pyridinyl or a pharmaceutically acceptable salt thereof.

6. A compound according to any one of claims 1 to 5 where Ar₂ is pyridinyl substituted with the group methyl and with a group selected from ethoxy, propoxy, phenyl, triazolyl, oxazolyl, thiazolyl, oxadiazolyl or pyrimidinyl, or a pharmaceutically acceptable salt thereof.

7. A compound according to any one of claims 1 to 6 where both Ar₁ and Ar₂ are pyridinyl, or a pharmaceutically acceptable salt thereof.

8. A compound according to any one of claims 1 to 7 where Ar₁ is pyridinyl substituted with —CF₃ and Ar₂ is pyridinyl substituted with the group methyl and with a group selected from phenyl, triazolyl, oxazolyl, thiazolyl, oxadiazolyl or pyrimidinyl, or a pharmaceutically acceptable salt thereof.

9. A compound of formula (I) selected from the group consisting of:
(1R,4S,6R)-3-{[6-Methyl-3-(propyloxy)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyrimidinyl]oxy}methyl)-3-azabicyclo[4.1.0]heptane;
(1R,4S,6R)-3-{[3-(ethyloxy)-6-methyl-2-pyridinyl]carbonyl}-4-({[5-(trifluoromethyl)-2-pyrimidinyl]oxy}methyl)-3-azabicyclo[4.1.0]heptane;
(1R,4S,6R)-3-[(6-methyl-3-phenyl-2-pyridinyl)carbonyl]-4-({[5-(trifluoromethyl)-2-pyrimidinyl]oxy}methyl)-3-azabicyclo[4.1.0]heptane;
(1R,4S,6R)-4-({[5-(methyloxy)-2-pyrimidinyl]oxy}methyl)-3-[(6-methyl-3-phenyl-2-pyridinyl)carbonyl]-3-azabicyclo[4.1.0]heptane;
(1R,4S,6R)-3-[(6-methyl-3-phenyl-2-pyridinyl)carbonyl]-4-({[6-(trifluoromethyl)-3-pyridazinyl]oxy}methyl)-3-azabicyclo[4.1.0]heptane;
(1R,4S,6R)-3-{[6-methyl-3-(2-pyrimidinyl)-2-pyridinyl]carbonyl}-4-({[6-(trifluoromethyl)-2-pyridinyl]oxy}methyl)-3-azabicyclo[4.1.0]heptane;
(1R,4S,6R)-4-{[(5-chloro-3-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-({[3-fluoro-5-(trifluoromethyl)-2-pyridinyl]oxy}methyl)-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[3-(trifluoromethyl)-2-pyridinyl]oxy}methyl)-3-azabicyclo[4.1.0]heptane;
6-[({(1R,4S,6R)-3-[(6-methyl-3-phenyl-2-pyridinyl)carbonyl]-3-azabicyclo[4.1.0]hept-4-yl}methyl)oxy]-3-pyridinecarbonitrile;
(1R,4S,6R)-4-({[6-(methyloxy)-2-pyridinyl]oxy}methyl)-3-[(6-methyl-3-phenyl-2-pyridinyl)carbonyl]-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyrimidinyl]oxy}methyl)-3-azabicyclo[4.1.0]heptane;
(1R,4S,6R)-3-{[5-methyl-2-(2-pyrimidinyl)phenyl]carbonyl}-4-({[6-(trifluoromethyl)-3-pyridazinyl]oxy}methyl)-3-azabicyclo[4.1.0]heptane;
(1R,4S,6R)-4-{[(4,5-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(2,6-dichloro-4-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4,6-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[4-(trifluoromethyl)-2-pyrimidinyl]oxy}methyl)-3-azabicyclo[4.1.0]heptane;
(1R,4S,6R)-3-{[6-methyl-3-(2-pyrimidinyl)-2-pyridinyl]carbonyl}-4-({[2-(trifluoromethyl)-4-pyrimidinyl]oxy}methyl)-3-azabicyclo[4.1.0]heptane;
(1R,4S,6R)-3-{[6-methyl-3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[5-(trifluoromethyl)-2-pyrimidinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-{[(3-methyl-2-pyrazinyl)oxy]methyl}-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[6-(trifluoromethyl)-3-pyridazinyl]oxy}methyl)-3-azabicyclo[4.1.0]heptane;
2-[({(1R,4S,6R)-3-[(6-methyl-3-phenyl-2-pyridinyl)carbonyl]-3-azabicyclo[4.1.0]hept-4-yl}methyl)oxy]-1,3-benzoxazole;
(1R,4S,6R)-4-{[(5-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4-fluoro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-({[6-methyl-4-(trifluoromethyl)-2-pyridinyl]oxy}methyl)-3-azabicyclo[4.1.0]heptane;
(1R,4S,6R)-4-{[(6-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(3,5-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(4-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(5-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(3-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2H-1,2,3-triazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[3-(5-ethyl-1,3-oxazol-2-yl)-6-methyl-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(4-methyl-1,3-thiazol-2-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(3-methyl-1,2,4-oxadiazol-5-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyridinyl]oxy}methyl)-3-azabicyclo[4.1.0]heptane;
(1R,4S,6R)-3-[(6-methyl-3-phenyl-2-pyridinyl)carbonyl]-4-({[5-(trifluoromethyl)-2-pyridinyl]oxy}methyl)-3-azabicyclo[4.1.0]heptane;
(1R,4S,6R)-3-{[6-methyl-3-(1H-pyrazol-1-yl)-2-pyridinyl]carbonyl}-4-({[5-(trifluoromethyl)-2-pyridinyl]oxy}methyl)-3-azabicyclo[4.1.0]heptane;
(1R,4S,6R)-3-{[6-methyl-3-(2-pyrimidinyl)-2-pyridinyl]carbonyl}-4-({[5-(trifluoromethyl)-2-pyrazinyl]oxy}methyl)-3-azabicyclo[4.1.0]heptane;
3-methyl-1-{[(1R,4S,6R)-4-({[5-(trifluoromethyl)-2-pyridinyl]oxy}methyl)-3-azabicyclo[4.1.0]hept-3-yl]carbonyl}-5H-imidazo[5,1-c]isoindole;
(1R,4S,6R)-3-{[5-methyl-2-(2-pyrimidinyl)phenyl]carbonyl}-4-({[5-(trifluoromethyl)-2-pyridinyl]oxy}methyl)-3-azabicyclo[4.1.0]heptane;
(1R,4S,6R)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane;
(1S,4S,6S)-3-{[6-methyl-3-(2-pyrimidinyl)-2-pyridinyl]carbonyl}-4-({[4-(trifluoromethyl)-2-pyridinyl]oxy}methyl)-3-azabicyclo[4.1.0]heptane;
(1S,4S,6S)-3-{[3-(2-pyrimidinyl)-2-pyridinyl]carbonyl}-4-({[4-(trifluoromethyl)-2-pyridinyl]oxy}methyl)-3-azabicyclo[4.1.0]heptane;
(1S,4S,6S)-3-{[5-methyl-2-(2-pyrimidinyl)phenyl]carbonyl}-4-({[4-(trifluoromethyl)-2-pyridinyl]oxy}methyl)-3-azabicyclo[4.1.0]heptane;
(1R,4S,6S)-4-{2-[(5-fluoro-2-pyridinyl)oxy]ethyl}-3-{[6-methyl-3-(2H-1,2,3-triazol-2-yl)-2-pyridinyl]carbonyl}-3-azabicyclo[4.1.0]heptane;
(1S,4S,6S)-4-{2-[(5-fluoro-2-pyridinyl)oxy]ethyl}-3-{[6-methyl-3-(2-pyrimidinyl)-2-pyridinyl]carbonyl}-3-azabicyclo[4.1.0]heptane;
(1R,4S,6R)-3-[(6-methyl-2-pyridinyl)carbonyl]-4-({[5-(trifluoromethyl)-2-pyridinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyridinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyrimidinyl]oxy}methyl)-3-azabicyclo[4.1.0]heptane;
(1R,4S,6R)-3-[(3-chloro-6-methyl-2-pyridinyl)carbonyl]-4-({[5-(trifluoromethyl)-2-pyrazinyl]oxy}methyl)-3-azabicyclo[4.1.0]heptane;
(1R,4S,6R)-3-{[6-methyl-3-(2-pyrimidinyl)-2-pyridinyl]carbonyl}-4-({[6-(trifluoromethyl)-3-pyridazinyl]oxy}methyl)-3-azabicyclo[4.1.0]heptane;
2-methyl-6-{[((1R,4S,6R)-3-{[6-methyl-3-(2-pyrimidinyl)-2-pyridinyl]carbonyl}-3-azabicyclo[4.1.0]hept-4-yl)methyl]oxy}-3-pyridinecarbonitrile;
(1R,4S,6R)-4-{[(4,6-dimethyl-2-pyrimidinyl)oxy]methyl}-3-{[6-methyl-3-(2-pyrimidinyl)-2-pyridinyl]carbonyl}-3-azabicyclo[4.1.0]heptane;
(1R,4S,6R)-4-{[(5,6-dimethyl-2-pyrazinyl)oxy]methyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-(2-{[5-(trifluoromethyl)-2-pyridinyl]oxy}ethyl)-3-azabicyclo[4.1.0]heptane;
(1R,4S,6R)-3-[(3-chloro-6-methyl-2-pyridinyl)carbonyl]-4-{2-[(5-fluoro-2-pyridinyl)oxy]ethyl}-3-azabicyclo[4.1.0]heptane;
(1R,4S,6R)-4-{2-[(4,6-dimethyl-2-pyrimidinyl)oxy]ethyl}-3-{[6-methyl-3-(2-pyrimidinyl)-2-pyridinyl]carbonyl}-3-azabicyclo[4.1.0]heptane;
(1R,4S,6R)-3-{[6-methyl-3-(2-pyrimidinyl)-2-pyridinyl]carbonyl}-4-[2-(3-pyridinyloxy)ethyl]-3-azabicyclo[4.1.0]heptane;
(1S,4S,6S)-4-{2-[(5-fluoro-2-pyridinyl)oxy]ethyl}-3-{[6-methyl-3-(1H-pyrazol-1-yl)-2-pyridinyl]carbonyl}-3-azabicyclo[4.1.0]heptane;
(1S,4S,6S)-3-{[6-methyl-3-(2-pyrimidinyl)-2-pyridinyl]carbonyl}-4-({[4-methyl-5-(trifluoromethyl)-2-pyridinyl]oxy}methyl)-3-azabicyclo[4.1.0]heptane;
(1S,4S,6S)-3-{[6-methyl-3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1S,4S,6S)-4-{[(2,6-dichloro-4-pyridinyl)oxy]methyl}-3-{[6-methyl-3-(2-pyrimidinyl)-2-pyridinyl]carbonyl}-3-azabicyclo[4.1.0]heptane;
(1R,4S,6S)-4-{[(4,6-dichloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6S)-4-{[(4,6-dichloro-2-pyridinyl)oxy]methyl}-3-{[3-(2-pyrimidinyl)-2-pyridinyl]carbonyl}-3-azabicyclo[4.1.0]heptane;
(1R,4S,6S)-4-{[(4-chloro-2-pyridinyl)oxy]methyl}-3-{[6-methyl-3-(2-pyrimidinyl)-2-pyridinyl]carbonyl}-3-azabicyclo[4.1.0]heptane;
(1R,4S,6S)-4-{[(4-chloro-2-pyridinyl)oxy]methyl}-3-{[3-(2-pyrimidinyl)-2-pyridinyl]carbonyl}-3-azabicyclo[4.1.0]heptane;
(1R,4S,6S)-3-{[3-(2H-1,2,3-triazol-2-yl)-2-pyridinyl]carbonyl}-4-({[4-(trifluoromethyl)-2-pyridinyl]oxy}methyl)-3-azabicyclo[4.1.0]heptane;
(1R,4S,6S)-3-{[6-methyl-3-(2-pyrimidinyl)-2-pyridinyl]carbonyl}-4-({[5-(trifluoromethyl)-2-pyrazinyl]oxy}methyl)-3-azabicyclo[4.1.0]heptane;
(1R,4S,6R)-4-{[(5-fluoro-2-pyridinyl)oxy]methyl}-3-[(2-methyl-5-phenyl-1,3-thiazol-4-yl)carbonyl]-3-azabicyclo[4.1.0]heptane;
(1R,4S,6R)-4-{[(4-fluoro-2-pyridinyl)oxy]methyl}-3-[(2-methyl-5-phenyl-1,3-thiazol-4-yl)carbonyl]-3-azabicyclo[4.1.0]heptane;
(1R,4S,6R)-4-{[(6-fluoro-2-pyridinyl)oxy]methyl}-3-[(2-methyl-5-phenyl-1,3-thiazol-4-yl)carbonyl]-3-azabicyclo[4.1.0]heptane; and
(1R,4S,6R)-4-{[(5-chloro-3-fluoro-2-pyridinyl)oxy]methyl}-3-[(2-methyl-5-phenyl-1,3-thiazol-4-yl)carbonyl]-3-azabicyclo[4.1.0]heptane;
or a pharmaceutically acceptable salt thereof.

10. The compound as defined in any one of claims 1 to 9, or a pharmaceutically acceptable salt thereof, for use in therapy.

11. The compound as defined in any one of claims 1 to 9, or a pharmaceutically acceptable salt thereof, for use in the treatment of a disease or disorder where an antagonist of a human orexin receptor is required.

12. The compound according to claim 11, or a pharmaceutically acceptable salt thereof, wherein the disease or disorder is a sleep disorder, a depression or mood disorder, an anxiety disorder, a substance-related disorder or a feeding disorder.

13. The compound according to claim 12, or a pharmaceutically acceptable salt thereof, wherein the disease or disorder is a sleep disorder.

14. The compound according to claim 13, or a pharmaceutically acceptable salt thereof, wherein the sleep disorder is selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

15. Use of a compound as defined in any one of claims 1 to 9, or a pharmaceutically acceptable salt thereof, in the manufacture of a medicament for use in the treatment of a disease or disorder where an antagonist of a human orexin receptor is required.

16. Use according to claim 15 where the disease or disorder is a sleep disorder, a depression or mood disorder, an anxiety disorder, a substance-related disorder or a feeding disorder.

17. Use according to claim 16 wherein the disease or disorder is a sleep disorder.

18. Use according to claim 17 where the sleep disorder is selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

19. A method for the treatment of a disease or disorder where an antagonist of a human orexin receptor is required, in a subject in need thereof, comprising administering to said subject an effective amount of a compound as defined in any one claims 1 to 9, or a pharmaceutically acceptable salt thereof.

20. A method according to claim 19 where the disease or disorder is a sleep disorder, a depression or mood disorder, an anxiety disorder, a substance-related disorder or a feeding disorder.

21. A method according to claim 20 where the disease or disorder is a sleep disorder.

22. A method according to claim 21 where the sleep disorder is selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

23. A pharmaceutical composition comprising a) the compound as defined in any one of claims 1 to 9, or a pharmaceutically acceptable salt thereof, and b) one or more pharmaceutically acceptable carriers.
